(19) United States
(12) Patent Application Publication
(10) Pub. No.: US 2024/0390627 A1
(43) Pub. Date: Nov. 28, 2024

(54) A VENT ASSEMBLY FOR A RESPIRATORY THERAPY SYSTEM

(71) Applicant: ResMed Pty Ltd, Bella Vista, New South Wales (AU)

(72) Inventors: Lorenz EBERL, Bella Vista, New South Wales (AU); Nookarajesh Varma SANGADI, Bella Vista, New South Wales (AU); Luke Andrew STANISLAS, Bella Vista, New South Wales (AU); Hongjiang YU, Bella Vista, New South Wales (AU); Muditha Pradeep DANTANARAYANA, Bella Vista, New South Wales (AU)

(21) Appl. No.: 18/697,444

(22) PCT Filed: Sep. 30, 2022
(86) PCT No.: PCT/AU2022/051170

§ 371 Date: Mar. 29, 2024

(30) Foreign Application Priority Data
Oct. 1, 2021 (AU) 2021903140
Apr. 21, 2022 (AU) 2022901050

(51) Int. Cl. A61M 16/06

(52) U.S. Cl. CPC A61M 16/0616

(57) ABSTRACT

A vent assembly for a respiratory therapy system configured in use to convey a vent flow of gases exhaled by a patient from a first volume interior to the respiratory therapy system to ambient. The vent assembly comprises a vent base, a flexible membrane and a vent cap. The membrane being mounted to the vent base and spanning across a vent base aperture. The vent cap is also mounted to the vent base and is located downstream of the membrane relative to the vent flow. The vent cap being positioned in the path of the vent flow through a membrane aperture. In use, the pressure of gas in the first volume acts on the membrane causing the membrane to flex thereby varying a position of the membrane relative to the vent cap in order to control the vent flow.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use. An assembly of straps comprised as part of a patient interface may be referred to as headgear.

### 1.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as a “tube down” configuration.

### 1.2.3.1.4 Pressurised Air Conduit Used for Positioning/Stabilising the Seal-Forming Structure

An alternative type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

Patient interfaces incorporating headgear tubing may provide some advantages, for example avoiding a conduit connecting to the patient interface at the front of a patient's face, which may be unsightly and obtrusive.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

Table of noise of prior masks (ISO 17510-2:2007, 10 cmH2O pressure at 1 m)

| | | A-weighted | A-weighted | |
|---|---|---|---|---|
| | | sound power | sound pressure | |
| | | level dB(A) | dB(A) | Year |
| Mask name | Mask type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed | nasal | 32 (3) | 24 (3) | 2002 |
| Mirage | | | | |
| ActivaTM | | | | |
| ResMed | nasal | 30 (3) | 22 (3) | 2008 |
| Mirage | | | | |
| MicroTM | | | | |
| ResMed | nasal | 29 (3) | 22 (3) | 2008 |
| MirageTM | | | | |
| SoftGel | | | | |
| ResMed | nasal | 26 (3) | 18 (3) | 2010 |
| MirageTM FX | | | | |
| ResMed | nasal | 37 | 29 | 2004 |
| Mirage | pillows | | | |
| SwiftTM (*) | | | | |
| ResMed | nasal | 28 (3) | 20 (3) | 2005 |
| Mirage | pillows | | | |
| SwiftTM II | | | | |
| ResMed | nasal | 25 (3) | 17 (3) | 2008 |
| Mirage | pillows | | | |
| SwiftTM LT | | | | |
| ResMed AirFit | nasal | 21 (3) | 13 (3) | 2014 |
| P10 | pillows | | | |

(* one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a vent assembly for a respiratory therapy system, the vent assembly being configured in use to convey a vent flow of gases exhaled by a patient from a first volume interior to the respiratory pressure therapy system to ambient.

In one form, the vent assembly may comprise a membrane configured to flex thereby varying a position of the membrane in order to control the vent flow through one or more vent outlets to ambient. The membrane may comprise a non-planar portion contoured inwardly in the direction of vent flow, for example the non-planar portion may be substantially dome-shaped.

In certain forms the vent assembly may comprise a vent base having formed therein a vent base aperture. The vent assembly may further comprise a flexible membrane mounted within the vent assembly (for example to the vent base) and spanning across the vent base aperture, wherein the membrane has formed therein a membrane aperture to allow the vent flow to pass therethrough. The vent assembly may further comprise a vent cap connected to the vent base, wherein the vent cap is located downstream of the membrane relative to the vent flow, and wherein the vent cap is positioned in the path of the vent flow through the membrane aperture. In use, the pressure of gas in the first volume may act on the membrane such that changes in the pressure of the gas in the first volume causes the membrane to flex thereby varying a position of the membrane relative to the vent cap in order to control the vent flow through one or more vent outlets to ambient.

In examples: a) the membrane comprises a non-planar portion contoured inwardly in the direction of vent flow; b) the non-planar portion is substantially dome-shaped; c) a central region of the membrane comprises the membrane aperture and non-central regions of the membrane are impermeable to gas; d) the vent cap is mounted to the vent base to form the one or more vent outlets between the vent cap and the vent base around a periphery of the vent cap; e) the one or more vent outlets are formed as a plurality of apertures in the vent cap and wherein the membrane and vent cap are configured so that varying flex of the membrane varies the number of the plurality of apertures that are blocked by the membrane to restrict the vent flow of gas to ambient therethrough; f) the membrane and vent cap are configured so that, as the flex of the membrane increases, apertures of the plurality of apertures located closer to a periphery of the vent cap are blocked by the membrane prior to apertures of the plurality of apertures located further from the periphery of the vent cap; g) the membrane has formed therein a plurality of membrane apertures to allow the vent flow to pass therethrough; h) the vent cap is sealingly mounted to the vent base around a periphery of the vent cap; i) a side of the vent cap facing towards the membrane has a shape corresponding to a shape of the membrane when flexed; j) the membrane is mounted to the vent base around a perimeter of the membrane; k) the membrane is sealingly mounted to the vent base around the perimeter of the membrane; l) the vent assembly is configured to form part of a patient interface; m) the vent base is configured to connect to a portion of a plenum chamber of the patient interface; and/or n) the vent assembly is configured such that a vent flow rate of the vent flow of gases from the first volume to ambient is substantially constant for a range of pressures inside the first volume.

In certain forms, the vent assembly may comprise a plurality of flaps forming an aperture through which a flow of air can pass in use from an internal volume of the respiratory therapy system to surrounding ambient air. The plurality of flaps may be configured such that, in use, when the pressure inside the internal volume increases, the plurality of flaps move to a first configuration, and when the pressure inside the internal volume decreases, the plurality of flaps move to a second configuration. An area of the aperture may be greater in the second configuration compared to the first configuration.

Another aspect of the present technology comprises a vent assembly for a respiratory therapy system.

In certain forms, the vent assembly may comprise a membrane. The vent assembly may further comprise a plurality of flaps located at a central region of the membrane and forming an aperture through which a flow of air can pass in use from an internal volume of the respiratory therapy system to surrounding ambient air. The membrane and the plurality of flaps may be configured such that, in use when the pressure inside the internal volume increases, the plurality of flaps move relative to the membrane to a first configuration, and when the pressure inside the internal volume decreases, the plurality of flaps move relative to the membrane to a second configuration. An area of the aperture may be greater in the second configuration compared to the first configuration.

In certain forms, the vent assembly may comprise a membrane having a concave inner surface and a convex outer surface. The vent assembly may further comprise a plurality of flaps located at a central region of the membrane and forming an aperture through which a flow of air can pass in use from an internal volume of the respiratory therapy system to surrounding ambient air. The membrane may be arranged with the concave inner surface facing the internal volume of the respiratory therapy system and the convex outer surface facing the surrounding ambient air. The membrane and the plurality of flaps may be configured such that, in use when the pressure inside the internal volume increases, the plurality of flaps move relative to the membrane to a first configuration, and when the pressure inside the internal volume decreases, the plurality of flaps move relative to the membrane to a second configuration. An area of the aperture may be greater in the second configuration compared to the first configuration. At least an upstream portion of the membrane may be positioned further from the centre of the aperture compared to a downstream portion of the membrane, the upstream portion being located upstream in relation to the flow of air and the downstream portion being located downstream in relation to the flow of air.

In examples: a) the plurality of flaps extend from the membrane in a radially inwards direction towards the aperture; b) the plurality of flaps are located around an outer circumference of the central aperture; c) the plurality of flaps have a curved shape; d) the curvature of a surface of the flaps contiguous with the inner surface of the membrane is greater than a curvature of a surface of the flaps contiguous with the outer surface of the membrane; e) the flaps have a thickness that varies along its length; f) the flaps have a greater thickness at a radially outer region compared to a radially inner region; g) the downstream portions of the flaps have a greater thickness than the upstream portions; h) the membrane comprises the plurality of flaps; i) the membrane defines a plurality of slits forming the plurality of flaps therebetween; j) the slits are oriented radially with respect to the membrane; k) the flaps are formed from a flexible and resilient material; l) the membrane is formed from a flexible and resilient material; m) the membrane is substantially dome-shaped; n) an outer region of the membrane connects to a vent base; o) the vent assembly is comprised as a part of a patient interface and the vent base connects to a portion of the patient interface; p) the vent base connects to a portion of a plenum chamber of the patient interface; q) the vent base connects to a portion of an elbow; r) the vent base is formed from a material that is relatively inflexible compared to the membrane; s) the vent assembly further comprises a vent cap; t) the vent cap is located downstream of the membrane; u) an inner surface of the vent cap has a curvature that is similar to the curvature of the outer surface of the membrane; v) the vent cap is formed from a material that is relatively inflexible compared to the membrane; w) the vent assembly further comprises a vent outlet located adjacent a radially outer region of the vent cap; x) the vent outlet is formed by one or more gaps between the vent cap and the vent base; y) the vent assembly is configured so that the flow of air through the aperture in the second configuration is approximately equal to the flow of air through the aperture in the first configuration; and/or z) the vent assembly is configured so that the flow of air through the aperture is approximately equal for a range of pressures of air in the internal volume in use.

In certain forms, the vent assembly may comprise a plurality of flaps forming an aperture through which a flow of air can pass in use from an internal volume of the respiratory therapy system to surrounding ambient air. The plurality of flaps may be separated by a plurality of slits. The plurality of flaps may be configured such that, in use, when the pressure inside the internal volume increases, the plurality of flaps move to a first configuration, and when the pressure inside the internal volume decreases, the plurality of flaps move to a second configuration. An area of the aperture may be greater in the second configuration compared to the first configuration.

In certain forms, the vent assembly may comprise a membrane, and a plurality of flaps located at a central region of the membrane and forming an aperture through which a flow of air can pass in use from an internal volume of the respiratory therapy system to surrounding ambient air. The membrane may define a plurality of slits forming the plurality of flaps therebetween. The membrane and the plurality of flaps may be configured such that, in use when the pressure inside the internal volume increases, the plurality of flaps move relative to the membrane to a first configuration, and when the pressure inside the internal volume decreases, the plurality of flaps move relative to the membrane to a second configuration. An area of the aperture may be greater in the second configuration compared to the first configuration.

In examples: a) the slits are oriented radially with respect to the membrane; and/or b) the membrane has a concave inner surface and a convex outer surface, and wherein the membrane is arranged with the concave inner surface facing the internal volume of the respiratory therapy system and the convex outer surface facing the surrounding ambient air.

In certain forms, the vent assembly may comprise a membrane, having a concave inner surface and a convex outer surface. The vent assembly may further comprise a plurality of flaps located at a central region of the membrane and forming an aperture through which a flow of air can pass in use from an internal volume of the respiratory therapy system to surrounding ambient air. The membrane may be arranged with the concave inner surface facing the internal volume of the respiratory therapy system and the convex outer surface facing the surrounding ambient air. The membrane and the plurality of flaps may be configured such that, in use, when the pressure inside the internal volume increases, the plurality of flaps move relative to the membrane to a first configuration and when the pressure inside the internal volume decreases, the plurality of flaps move relative to the membrane to a second configuration. An area of the aperture may be greater in the second configuration compared to the first configuration. At least a portion of the membrane may be located upstream, in relation to the flow of air, of the aperture.

Another aspect of one form of the present technology comprises a vent assembly for a respiratory therapy system for providing respiratory pressure therapy to a patient, the vent assembly being configured in use to convey a vent flow of gases from a first volume interior to the respiratory therapy system to ambient, the vent assembly comprising:
a vent body fluidly connected in use to the first volume and comprising a vent body aperture through which the vent flow of gases flows in use towards ambient;
a plunger positioned with respect to the vent body to define a regulated vent flow passage for the vent flow of gases between the plunger and the vent body;
wherein the plunger is movable with respect to the vent body and biased towards a rest position in which the regulated vent flow passage is open;
wherein, in use, changes in pressure of gas in the first volume cause changes in a position of the plunger relative to the vent body to regulate the vent flow of gases through the regulated vent flow passage throughout a therapeutic pressure range.
   In examples:
the vent assembly is configured to convey the vent flow of gases from the first volume to atmosphere continuously throughout a respiratory cycle of the patient;
a frustoconical portion of the plunger and a frustoconical portion of the vent body together define the regulated vent flow passage between the plunger and the vent body;
wherein in use when the position of the plunger relative to the vent body changes, the frustoconical portion of the plunger moves with respect to the frustoconical portion of the vent body to change a cross-sectional area of the regulated vent flow passage;
the vent assembly comprises a connecting portion movably connecting the plunger to the vent body;
the connecting portion comprises at least one flexible membrane supported by vent body, the plunger being attached to the membrane, wherein changes in pressure of gas in the first volume cause deformation of the membrane causing movement of the plunger;
the membrane is connected to the vent body about a periphery of the membrane, the plunger is attached to the centre of the membrane and the membrane comprises membrane apertures through which the vent flow of gases is able to flow from the first volume towards the regulated gas passage;
upon deformation of the membrane the plunger moves in a direction aligned with a central axis of the vent body;
the plunger and the membrane each comprise a central axis aligned with the central axis of the vent body;
the vent assembly comprises a diffuser contained within the vent body and positioned such that the vent flow of gases is incident on the diffuser downstream of the vent body aperture;
the vent assembly comprises a diffuser cover attached to a downstream side of the vent body and configured to retain the diffuser in the vent body;
the vent assembly comprises one or more fixed size apertures defining one or more unregulated vent flow passages in addition to the regulated vent flow passages;
the vent assembly further comprises an upstream cover portion attached to the vent body and configured to cover the membrane upstream of the membrane; and/or
the plunger and membrane are integrally formed.

In further examples:
the regulated vent flow passage comprises an upstream portion and a downstream portion, the downstream portion being shaped to have a cross-sectional area that enlarges in the downstream direction independent of movement or position of the plunger;
the vent body comprises an upstream body portion and a downstream body portion, the upstream body portion defining the vent body aperture and the downstream body portion at least partially defining the downstream portion of the regulated vent flow passage;
the vent body comprises opposing divergent surfaces defining the downstream portion of the regulated vent flow passage, the divergent surfaces diverging in the downstream direction such that the cross-sectional area of the downstream portion of the regulated vent flow passage increases in the downstream direction;
the plunger extends through the vent body aperture and partially defines the downstream portion of the regulated vent flow passage;
the connecting portion comprises an upstream membrane attached to an upstream end of the plunger and a downstream membrane attached to a downstream end of the plunger, each of the upstream membrane and the downstream membrane being connected to the vent body;
the vent body encloses the upstream membrane and the downstream membrane;
the vent body comprises a first lateral side portion and a second lateral side portion opposing and connected to the first lateral side portion, the first lateral side portion and second lateral side portion together defining the vent body aperture;
the first lateral side portion and the second lateral side portion together define a circumferential outer surface of the vent body;
the vent body comprises a groove formed in the circumferential outer surface configured to receive a portion of a patient interface, enabling connection of the vent assembly to the patient interface; and/or
the first lateral side portion and the second lateral side portion comprise complementary snap fit features configured to enable the first lateral side portion and the second lateral side portion to snap fit together.

In further examples:
the connecting portion comprises a spring provided between the plunger and the vent body;
the spring comprises a coil spring;
the plunger comprises a central recess, the spring is positioned within the central recess and is seated against a downstream end of the vent body;
the spring comprises a bellows spring;
the bellows spring is integrally formed with the plunger and extends from a downstream side of the plunger and is seated against a downstream end of the vent body;
the regulated vent flow passage comprises an upstream portion and a downstream portion, the downstream portion being shaped to have a cross sectional area that enlarges in the downstream direction independent of movement or position of the plunger, the bellows spring partially defining the downstream portion of the regulated vent flow passage;
the vent assembly comprises an upstream cover portion configured to at least partially cover the plunger upstream of the plunger;
the vent assembly comprises an upstream cover portion configured to at least partially cover the plunger upstream of the plunger and comprising a central pin extending in a downstream direction through a central hole in the plunger to a downstream end of the vent assembly, the central pin comprising a flange positioned at the downstream end of the vent assembly, the bellows spring being integrally formed with the plunger and being seated against the flange;
the connecting portion comprises an expandable bellows spring, and wherein, in use, pressure of gas in the first volume causes the expandable bellows spring to expand to move the plunger towards the vent body to regulate the vent flow of gases through the regulated vent flow passage throughout the therapeutic pressure range;
the vent body aperture is provided around an outer circumference of the vent body;
the plunger is provided adjacent the outer circumference of the vent body;
the plunger is integrally formed with the expandable bellows spring;
the expandable bellows spring comprises a disc portion defining an end of the vent assembly opposite the upstream end of the vent assembly; and/or
the disc portion is attached to a central pin attached to the vent body.

In further examples:
the vent assembly comprises a first magnetic portion and a second magnetic portion, one of the first magnetic portion and the second magnetic portion comprises a magnet and the other of the first magnetic portion and the second magnetic portion comprises a magnet or comprises a ferromagnetic material, the first magnetic portion being supported within the vent assembly and the second magnetic portion being attached to the plunger, wherein a magnetic force acts between first magnetic portion and the second magnetic portion biasing the plunger towards the rest position;
in use the plunger moves away from the first magnetic portion to regulate the vent flow of gas through the regulated vent flow passage, and the magnetic force is an attractive force;
both of the first magnetic portion and the second magnetic portion comprise magnets;
the vent assembly comprises an upstream cover portion attached to the vent body and configured to cover the plunger upstream of the plunger, the first magnetic portion being retained by the upstream cover portion;
the first magnetic portion is mounted to an upstream side of the upstream cover portion; and/or
the plunger comprises a central recess, the second magnetic portion being retained within the central recess.

Another aspect of one form of the present technology comprises a vent assembly for a respiratory therapy system for providing respiratory pressure therapy to a patient, the vent assembly being configured in use to convey a vent flow of gases from a first volume interior to the respiratory therapy system to ambient, the vent assembly comprising:
a vent body fluidly connected in use to the first volume and comprising a vent body aperture through which the vent flow of gases flows in use towards ambient;
a moveable portion positioned with respect to the vent body to define a regulated vent flow passage for the vent flow of gases between the moveable portion and the vent body, the moveable portion being movable with respect to the vent body and biased towards a rest position in which the regulated vent flow passage is open;
a first magnetic portion and a second magnetic portion, one of the first magnetic portion and the second magnetic portion being a magnet and the other of the first magnetic portion and the second magnetic portion being a magnet or being formed from a ferromagnetic material, the first magnetic portion being supported within the vent assembly and the second magnetic portion being provided to the moveable portion, wherein a magnetic force acts between first magnetic portion and the second magnetic portion biasing the movable portion towards the rest position;
wherein, in use, changes in pressure of gas in the first volume cause changes in a position of the moveable portion relative to the vent body to regulate the vent flow of gases through the regulated vent flow passage throughout a therapeutic pressure range.

In examples:
in use the moveable portion moves away from the first magnetic portion to regulate the vent flow of gas through the regulated vent flow passage and the magnetic force is an attractive force;
both of the first magnetic portion and the second magnetic portion comprise magnets;
in use the moveable portion moves towards the first magnetic portion to regulate the vent flow of gas through the regulated vent flow passage and the magnetic force is a repulsive force;
the vent assembly comprises an upstream cover portion attached to the vent body and configured to cover the moveable portion upstream of the moveable portion, the first magnetic portion being retained by the upstream cover portion;
the first magnetic portion is mounted to an upstream side of the upstream cover portion;
the moveable portion comprises a central recess, the second magnetic portion being retained within the central recess; and/or
the moveable portion comprises a plunger.

Another aspect of one form of the present technology comprises a vent assembly for a respiratory therapy system for providing respiratory pressure therapy to a patient, the vent assembly being configured in use to convey a vent flow of gases from a first volume interior to the respiratory therapy system to ambient, the vent assembly comprising:
a vent body fluidly connected to the first volume and defining one or more vent outlets;
an annular membrane mounted within the vent assembly;
wherein a regulated vent flow passage is formed between a moveable edge of the membrane and a membrane-adjacent portion of the vent body through which the vent flow of gases passes during flow from the first volume to the one or more vent outlets;
wherein in use, changes in pressure of gas in the first volume cause changes in a position of some or all of the moveable edge of the membrane relative to the membrane-adjacent portion of the vent body to regulate the vent flow of gases through the regulated vent flow passage throughout a therapeutic pressure range;
wherein at least some of the vent flow of gases is able to flow from the regulated vent flow passage along a straight-line path to and through the one or more vent outlets, the straight-line path being unimpeded or impeded only by one or more diffusers.

In examples:
the moveable edge is an inner edge of the membrane and the membrane comprises an outer edge;
the membrane comprises a frustoconical portion;
the membrane comprises a bead formed at the moveable edge;
the vent body comprises an annular rib forming the membrane-adjacent portion of the vent body;
the vent body comprises a plurality of stops sized and positioned to limit movement of the moveable edge of the membrane in use towards the membrane-adjacent portion, the regulated vent flow passage formed by gaps between the stops;
the vent body comprises a membrane retainer portion supporting the outer edge of the membrane, and a vent cap attached to the membrane retainer portion;
the vent cap forms the membrane-adjacent portion of the vent body;
the one or more vent outlets comprises a plurality of vent outlets,
at least some of the plurality of vent outlets open radially outward;
at least some of the plurality of vent outlets are defined partially by the membrane retainer portion of the vent body and partially by the vent cap;
at least some of the plurality of vent outlets are formed in the vent cap and are spaced inwardly from an outermost-periphery of the vent body;
the vent cap defines the plurality of vent outlets;
the vent assembly comprises a diffuser retained between the membrane, the membrane retainer portion and the vent cap, the diffuser positioned such that at least some of the vent flow of gases is able to flow from the regulated vent flow passage along the straight-line path to the diffuser and then, upon exit from the diffuser, along the straight-line path to the vent outlets;
the moveable edge of the membrane is an outer edge and the membrane further comprises an inner edge;
the vent body comprises a membrane retainer portion supporting the inner edge;
the membrane retainer portion forms the membrane-adjacent portion of the vent body;
the membrane-adjacent portion is formed by an inner peripheral edge of the membrane retainer portion;
the one or more vent outlets comprises a plurality of vent outlets.
at least some of the plurality of vent outlets open radially outward;
the vent outlets which open radially outward are formed by the membrane retainer portion;
at least some of the plurality of vent outlets open in a direction parallel to a central axis of the membrane;
the vent body comprises a vent cap attached to the membrane retainer portion;
the vent cap forms at least some of the plurality of vent outlets;
the vent body comprises an annular membrane cover, the inner edge of the membrane being held between the membrane retainer portion and the annular membrane cover;
the vent assembly comprises one or more fixed size apertures defining one or more unregulated vent flow passages in addition to the regulated vent flow passage;
the vent assembly is configured to fluidly connect to a plenum chamber of a patient interface, the vent assembly comprising an air inlet configured to receive a pressurised flow of gas at the therapeutic pressure for supply to the plenum chamber for breathing by the patient;
the vent body comprises a connector for connecting to an air circuit of the respiratory therapy system, the connector defining the air inlet and positioned centrally with respect to the vent assembly and configured to project away from the plenum chamber in use; and/or
the vent assembly comprises a heat and moisture exchanger attached to the vent body.

Another aspect of one form of the present technology comprises a vent assembly for a respiratory therapy system for providing respiratory pressure therapy to a patient, the vent assembly being configured in use to convey a vent flow of gases from a first volume interior to the respiratory therapy system to ambient, the vent assembly comprising:
a vent body fluidly connected to the first volume and defining one or more vent outlets;
an annular membrane mounted within the vent assembly and comprising an inner edge and an outer edge, and the annular membrane further comprising at least one membrane aperture formed in the membrane between the inner edge and the outer edge;
wherein a first regulated vent flow passage is formed between a first membrane-adjacent portion of the vent body and the membrane aperture, the vent flow of gases being able to pass through the first regulated vent flow passage during flow from the first volume to the one or more vent outlets;
wherein in use, changes in pressure of gas in the first volume cause changes in a position of the at least one membrane aperture relative to the first membrane-adjacent portion of the vent body to regulate the vent flow of gases through the first regulated vent flow passage throughout the therapeutic pressure range.

In examples:
the one or more vent outlets comprises at least one annular vent outlet;
the one or more vent outlets comprises a pair of annular vent outlets and the first membrane-adjacent portion of the vent body is provided between the pair of annular vent outlets;
the membrane comprises a plurality of membrane apertures spaced along a circumference of the membrane;
the circumference of the membrane along which the membrane apertures are spaced is centrally located between the inner edge and the outer edge of the membrane;
each of the plurality of membrane apertures comprises a slit;
each slit is aligned along the circumference of the membrane along which the membrane apertures are spaced;
one of the inner edge and the outer edge is a moveable edge of the membrane; and
   wherein a second regulated vent flow passage is formed between a second membrane-adjacent portion of the vent body and the moveable edge of the membrane, the vent flow of gases being able to pass through the second regulated vent flow passage during flow from the first volume to the one or more vent outlets;
   wherein in use, changes in pressure of gas in the first volume cause changes in a position of some or all of the moveable edge of the membrane relative to the second membrane-adjacent portion of the vent body to regulate the vent flow of gases through the second regulated vent flow passage throughout a therapeutic pressure range;
at least below a predetermined pressure in the first volume, the vent flow of gases are able to flows through the first regulated vent flow passage and the second regulated vent flow passage in parallel;
upon pressure in the first volume exceeding the predetermined pressure, the second regulated vent flow passage closes;
the vent body comprises a cylindrical outer portion supporting the outer edge of the annular membrane and a cylindrical inner portion forming the second membrane-adjacent portion of the vent body;
the vent body comprises a vent base on which the membrane is supported and a vent cap defining the first membrane-adjacent portion of the vent body;
at least one of the vent outlets is formed in the vent cap;
at least one of the vent outlets is defined by a spacing between a peripheral edge of the vent cap and an outer edge of the membrane;
the first membrane-adjacent portion comprises a plurality of discrete surfaces separated by openings through which the vent flow of gas is able to flow after passing through the membrane apertures;
the vent assembly comprises a diffuser between the first regulated vent flow passage and the vent outlet;
each of the at least one membrane aperture is a circular hole;
the vent assembly comprises one or more fixed size apertures defining one or more unregulated vent flow passages; and/or
the fixed size aperture(s) are formed in the vent body in position(s) radially inward of the inner edge of the membrane.

Another aspect of one form of the present technology comprises a vent assembly for a respiratory therapy system for providing respiratory pressure therapy to a patient, the vent assembly being configured in use to convey a vent flow of gases from a first volume interior to the respiratory therapy system to ambient, the vent assembly comprising:
a vent body fluidly connected to the first volume and defining a vent flow passage through which the vent flow of gases is able to flow from the first volume to ambient,
wherein the vent flow passage comprises an upstream portion and a downstream portion, wherein at least the downstream portion is annular in cross section and is shaped to have a cross sectional area that enlarges in the downstream direction.

In examples:
the vent body comprises opposing divergent surfaces defining the downstream portion of the vent flow passage, the divergent surfaces diverging in the downstream direction such that the cross-sectional area of the downstream portion of the vent flow passage increases in the downstream direction;
the upstream portion of the vent flow passage has a substantially constant cross-sectional area along its length;
the upstream portion of the vent flow passage is defined by opposing parallel surfaces of the vent body;
the upstream portion of the vent flow passage is shaped such that the vent flow of gases flows partially radially inwardly along the length of the upstream portion in a downstream direction;
the downstream portion of the vent flow passage is shaped such that the vent flow of gases flows partially radially outwardly along the length of the downstream portion in the downstream direction;
the downstream portion of the vent flow passage is defined by opposing non-parallel surfaces;
each of the opposing non-parallel surfaces extends radially outwardly in the downstream direction;
the vent body comprises a central portion and a peripheral portion, the downstream portion of the vent flow passage being defined between the central portion and the peripheral portion; and/or
the vent body comprises an upstream cover portion, the upstream cover portion connecting between the central portion and the peripheral portion and supporting the central portion within the peripheral portion, the upstream portion of the vent flow passage being defined between the upstream cover portion and the peripheral portion.

Another aspect of certain forms of the technology is a patient interface for use in a respiratory therapy system. The patient interface may be configured to deliver a flow of gas at positive pressure to a patient's airways. The patient interface may comprise a vent assembly according to another aspect of the technology.

An aspect of one form of the present technology is a patient interface for use in a respiratory therapy system. The patient interface may comprise a plenum chamber pressurisable to a therapeutic pressure of at least 6 cmH2O above ambient air pressure, said plenum chamber including a plenum chamber inlet port sized and structured to receive a flow of air at the therapeutic pressure for breathing by a patient. The patient interface may further comprise a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use. The patient interface may also comprise a vent assembly according to another aspect of the present technology. The vent assembly may allow a flow of gases exhaled by the patient from an interior of the plenum chamber to ambient, said vent assembly being sized and shaped to maintain the therapeutic pressure in the plenum chamber in use. The patient interface may also be configured to allow the patient to breath from ambient through their mouth in the absence of a flow of pressurised air through the plenum chamber inlet port, or the patient interface is configured to leave the patient's mouth uncovered.

In some forms, the plenum chamber comprises the vent assembly.

An aspect of one form of the present technology is a respiratory therapy system comprising a patient interface according to another aspect of the present technology. The respiratory therapy system may further comprise at least one air circuit for supplying the flow of gas at the therapeutic pressure to the patient interface.

Another aspect of the technology is an air circuit for use in a respiratory therapy system. The air circuit may be configured to deliver a supply of gas from an RPT device to a patient interface. The air circuit may comprise a vent assembly according to another aspect of the technology.

An aspect of one form of the present technology is a respiratory therapy system comprising a vent assembly according to another aspect of the present technology. The respiratory therapy system may further comprise at least one air circuit for supplying the flow of gas at the therapeutic pressure to the patient interface. The respiratory therapy system may further comprise a patient interface. The patient interface may comprise a plenum chamber pressurisable to a therapeutic pressure of at least 6 cmH2O above ambient air pressure, said plenum chamber including a plenum chamber inlet port sized and structured to receive a flow of air at the therapeutic pressure for breathing by a patient. The patient interface may further comprise a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use. The patient interface may be configured to allow the patient to breath from ambient through their mouth in the absence of a flow of pressurised air through the plenum chamber inlet port, or the patient interface may be configured to leave the patient's mouth uncovered.

An aspect of one form of the present technology is a respiratory therapy system comprising at least one air circuit for supplying the flow of gas at the therapeutic pressure to the patient interface. The respiratory therapy system may further comprise a patient interface. The patient interface may comprise a plenum chamber pressurisable to a therapeutic pressure of at least 6 cmH2O above ambient air pressure, said plenum chamber including a plenum chamber inlet port sized and structured to receive a flow of air at the therapeutic pressure for breathing by a patient. The patient interface may further comprise a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use. The patient interface may be configured to allow the patient to breath from ambient through their mouth in the absence of a flow of pressurised air through the plenum chamber inlet port, or the patient interface may be configured to leave the patient's mouth uncovered.

In some forms, the air circuit comprises a vent assembly according to another aspect of the present technology.

Another aspect of the technology is an elbow for use in a respiratory therapy system. The elbow may be configured to deliver a supply of gas from an air circuit to a plenum chamber of a patient interface. The elbow may be comprised as part of the patient interface or may connect to the patient interface in use. The elbow may comprise a vent assembly according to another aspect of the technology.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

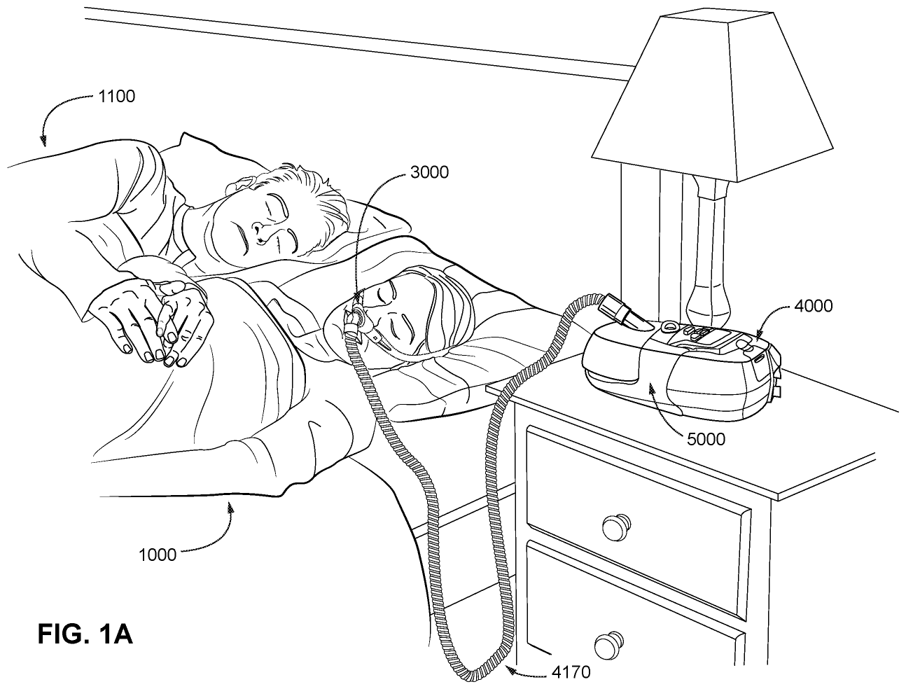

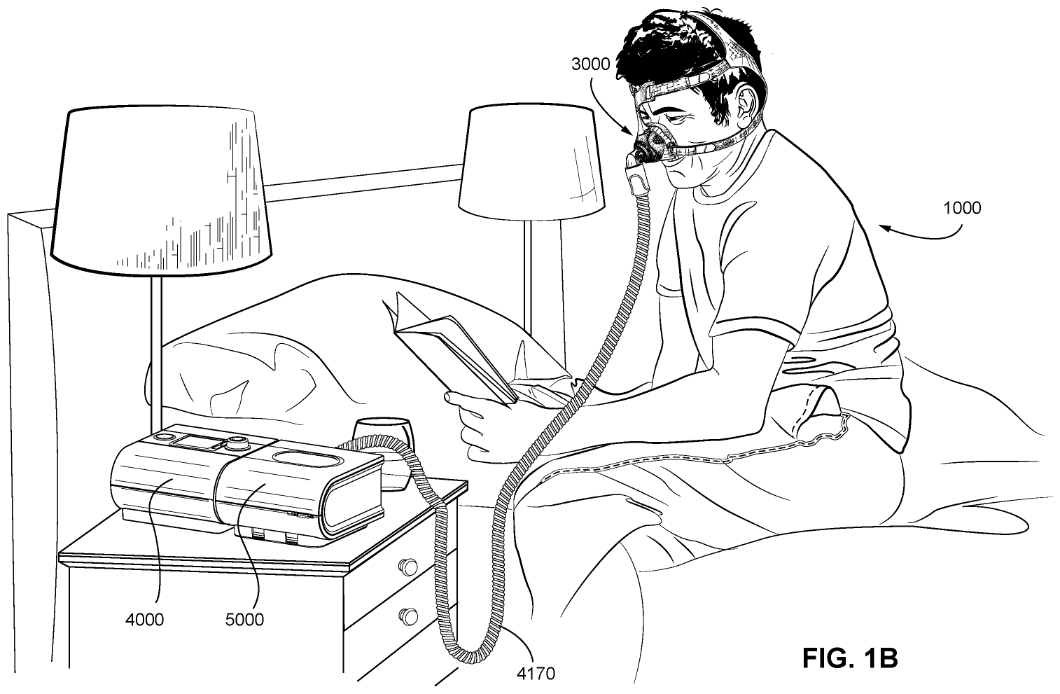

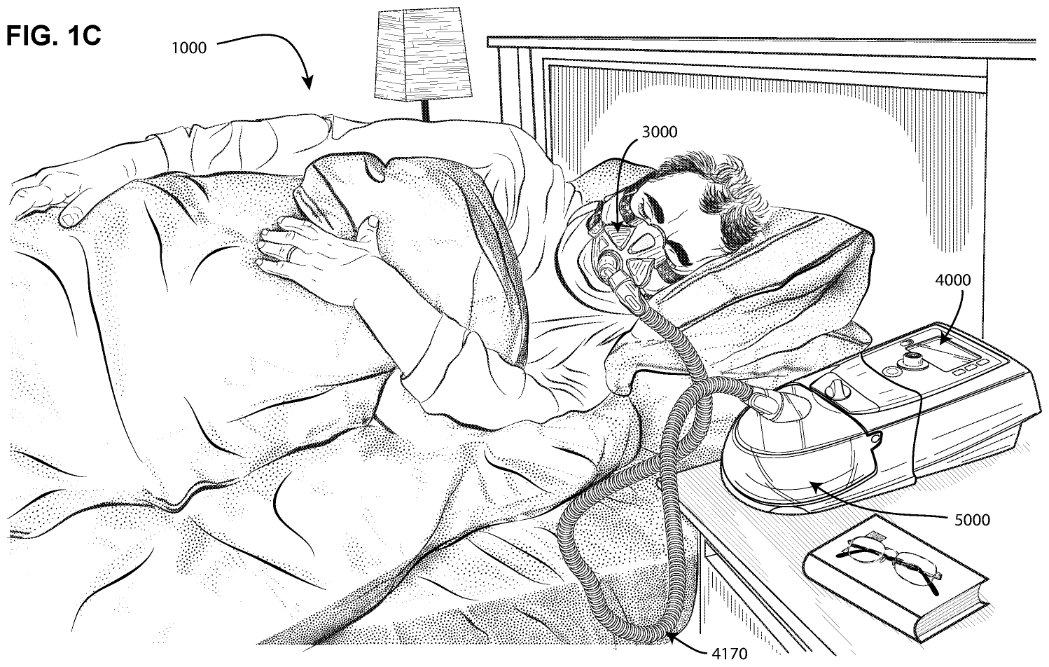

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

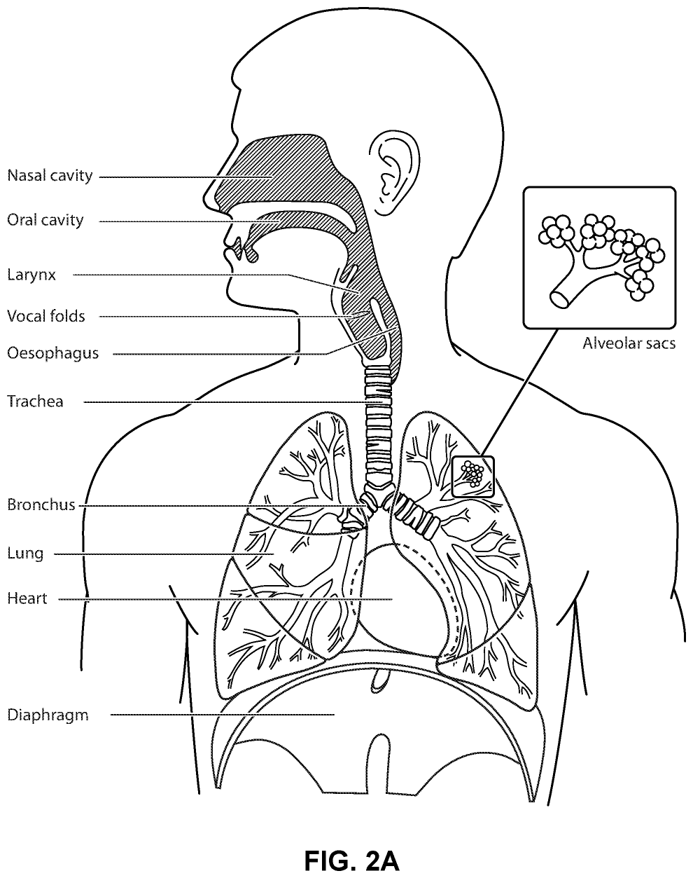

FIG. 2A shows an overview of a human respiratory system including the nasal and oral cavities, the larynx, vocal folds, oesophagus, trachea, bronchus, lung, alveolar sacs, heart and diaphragm.

### 3.3 Patient Interface

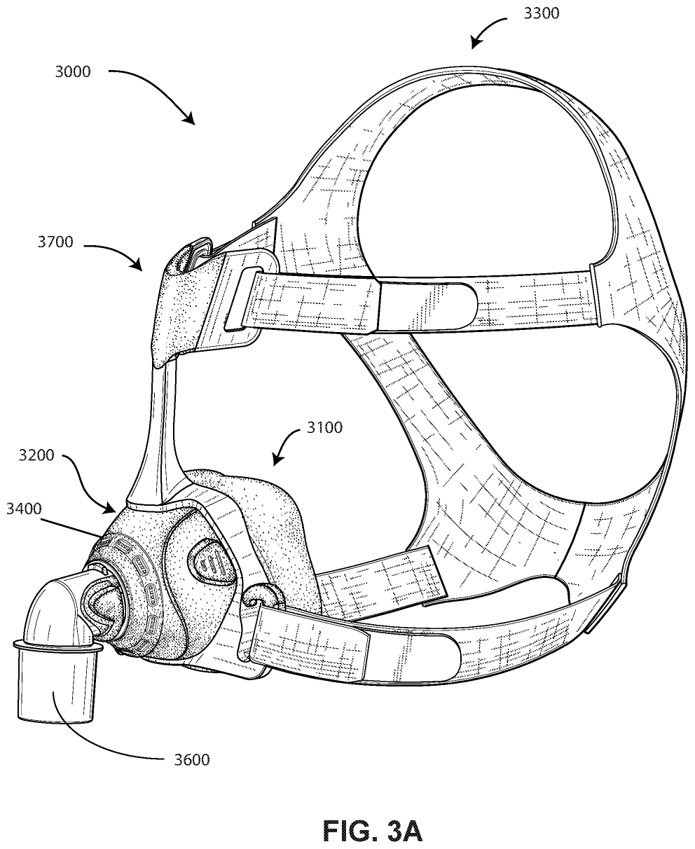

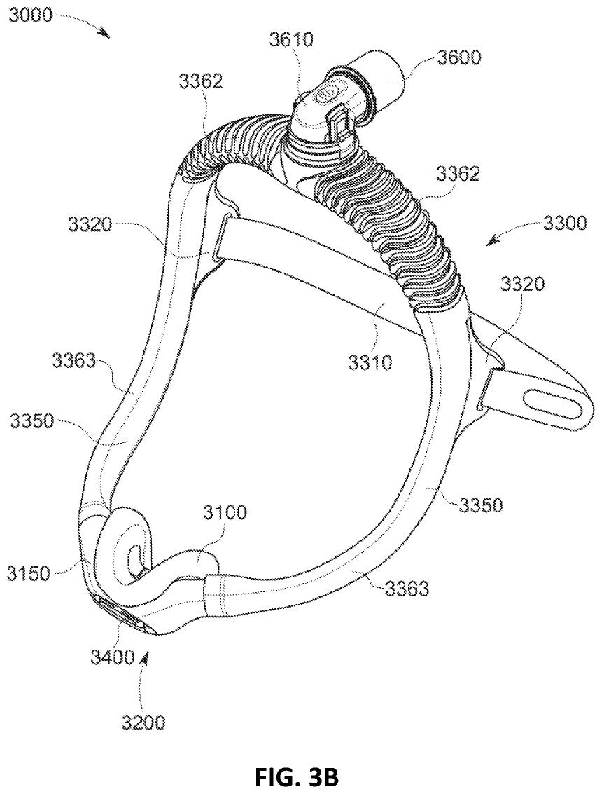

FIG. 3A shows a patient interface in the form of a nasal mask in accordance with one form of the present technology.

FIG. 3B shows a patient interface in the form of a mask having conduit headgear in accordance with one form of the present technology.

### 3.4 RPT Device

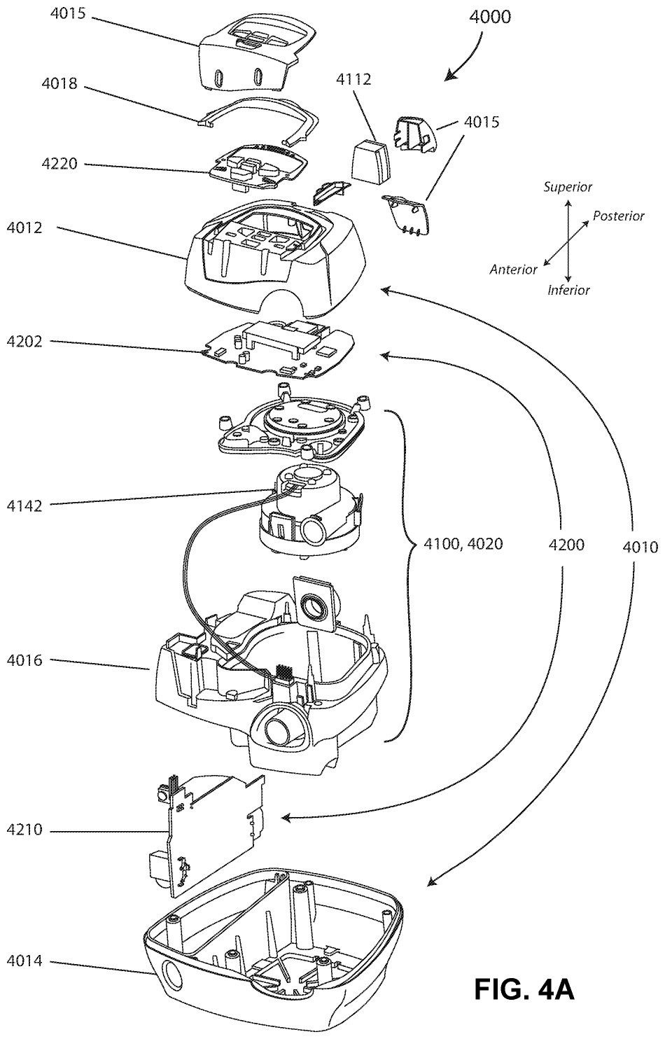

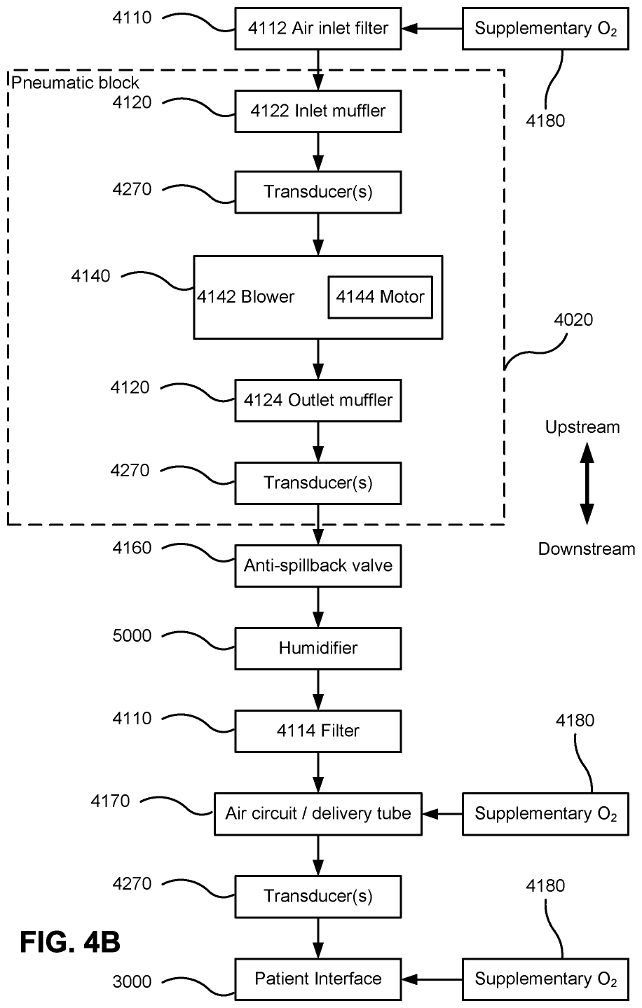

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

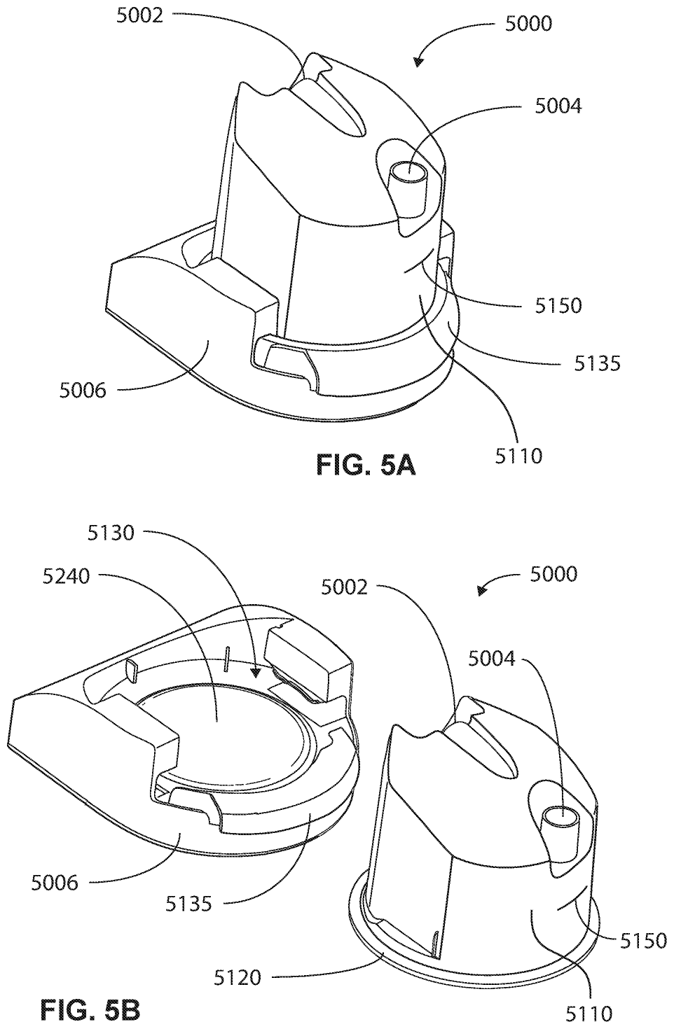

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Vent Assembly

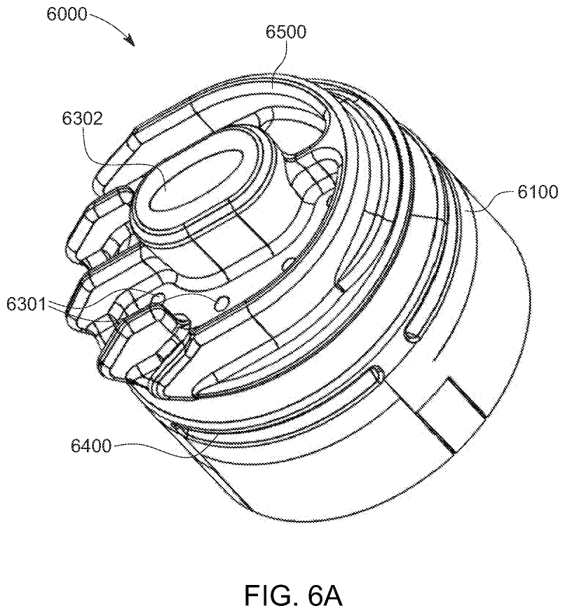

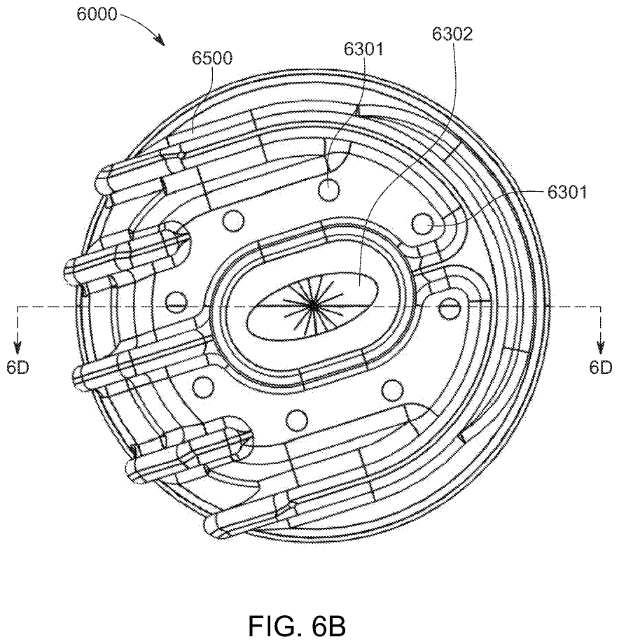

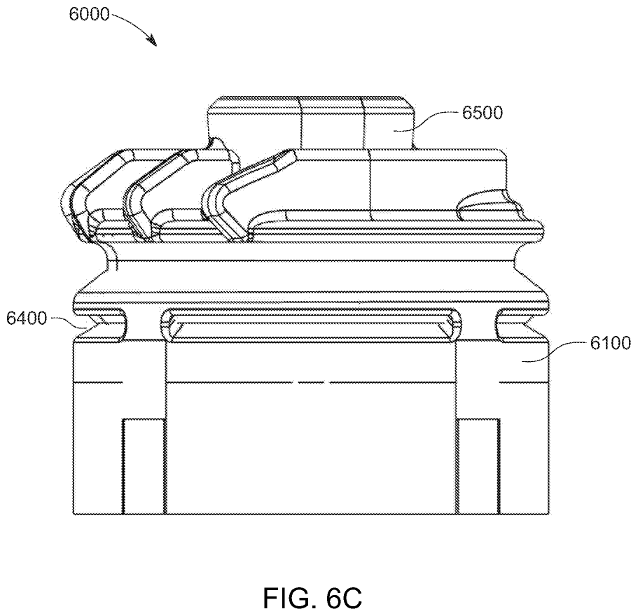

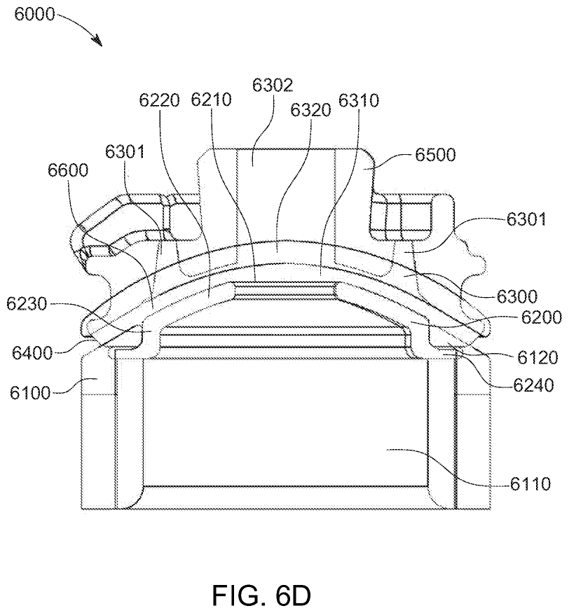

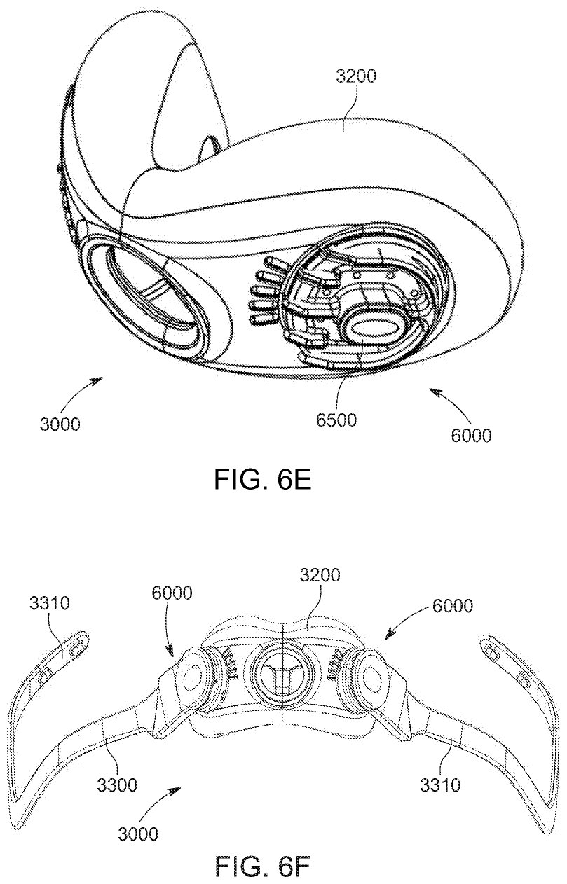

FIG. 6A shows a perspective view of a vent assembly according to one aspect of the present technology.

FIG. 6B shows a top view of the vent assembly of FIG. 6A.

FIG. 6C shows a side view of the vent assembly of FIG. 6A.

FIG. 6D shows a cross-sectional view of the vent assembly along the plane A-A of FIG. 6B.

FIG. 6E shows a perspective view of a part of a patient interface including the vent assembly of FIG. 6A connected to a plenum chamber.

FIG. 6F shows a perspective view of a part a patient interface including the vent assembly of FIG. 6A connected to both a plenum chamber and a positioning and stabilising structure.

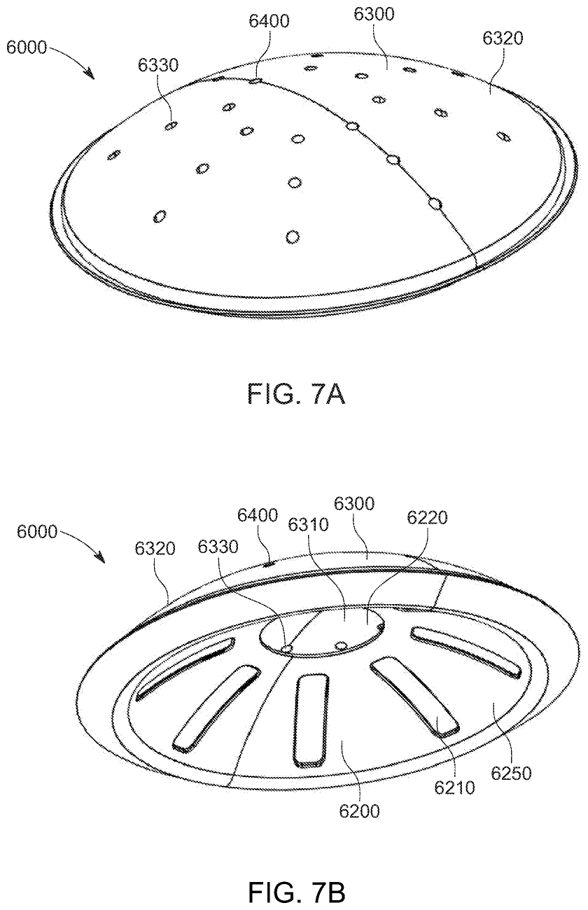

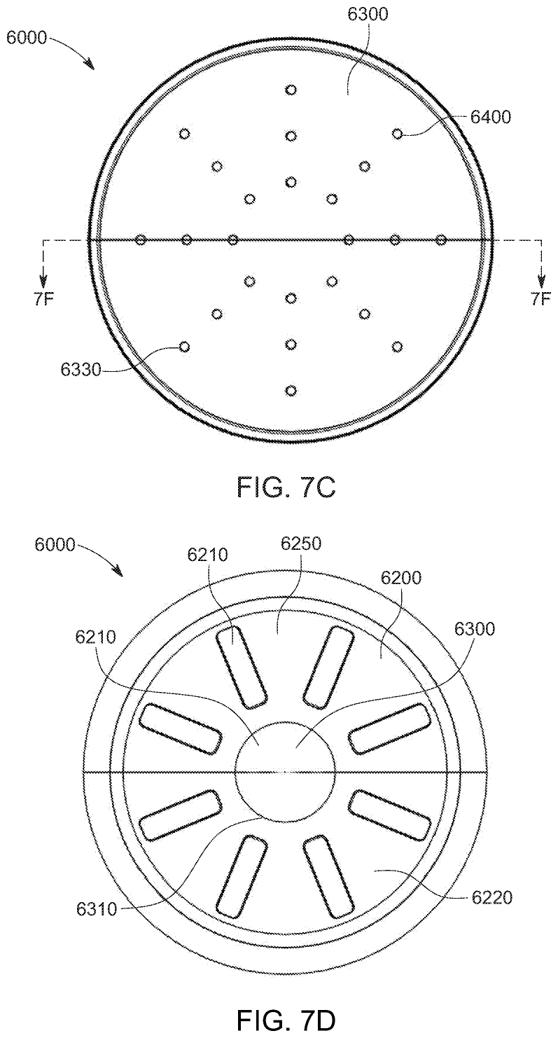

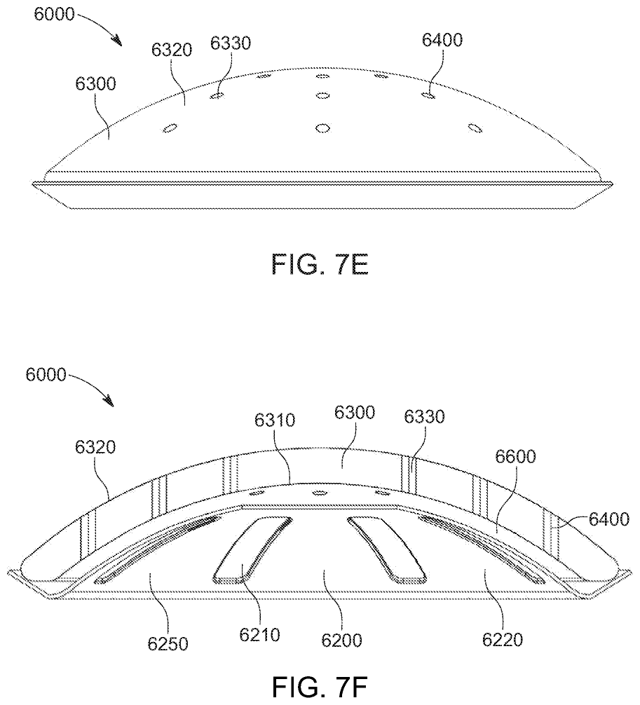

FIG. 7A shows a top perspective view of a vent assembly according to another aspect of the present technology.

FIG. 7B shows a bottom perspective view of the vent assembly of FIG. 7A.

FIG. 7C shows a top view of the vent assembly of FIG. 7A.

FIG. 7D shows a bottom view of the vent assembly of FIG. 7A.

FIG. 7E shows a side view of the vent assembly of FIG. 7A.

FIG. 7F shows a cross-sectional view of the vent assembly along the plane B-B of FIG. 7C.

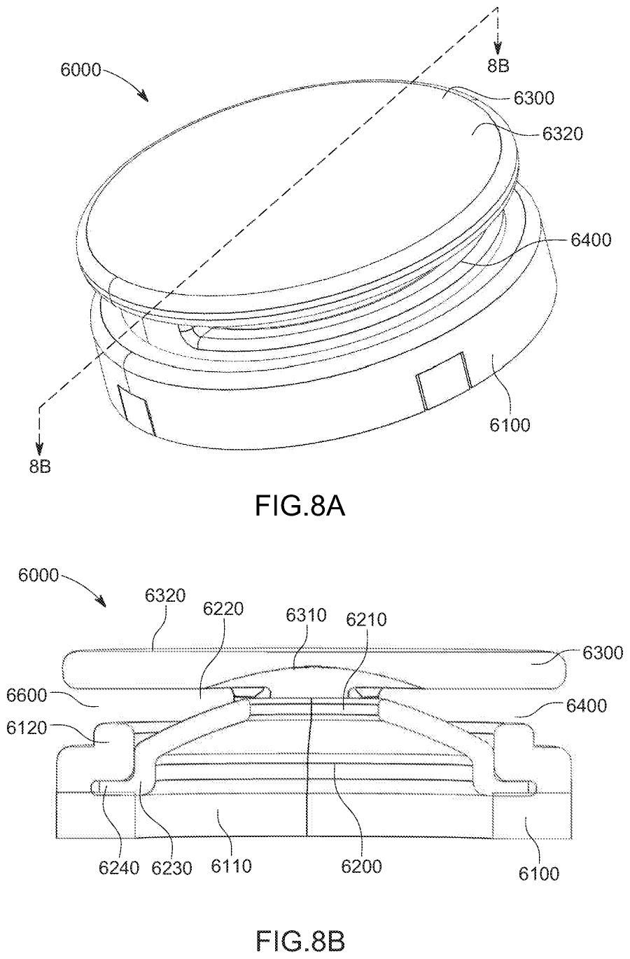

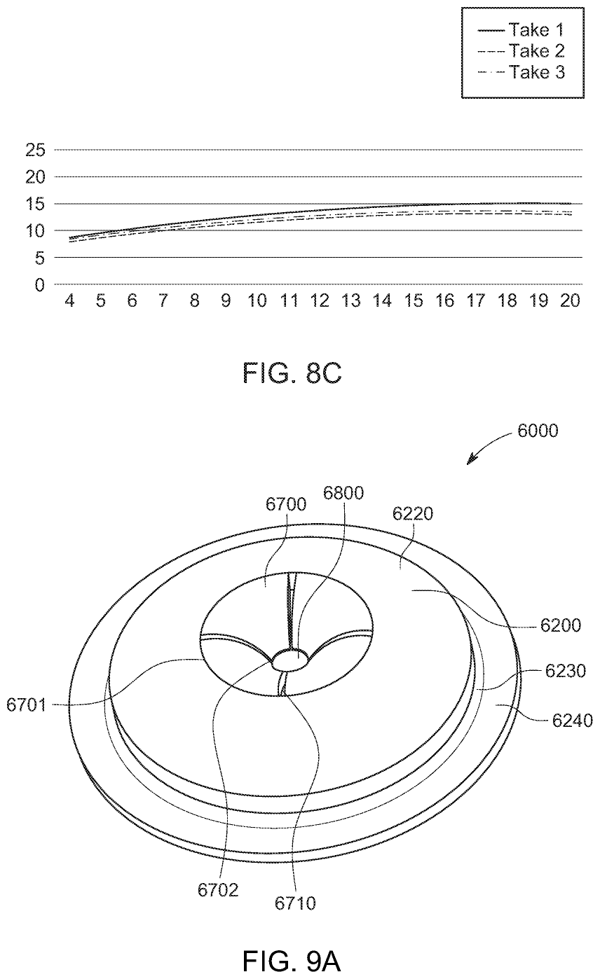

FIG. 8A shows a perspective view of a vent assembly according to another aspect of the present technology.

FIG. 8B shows a cross-sectional view of the vent assembly along the plane C-C of FIG. 8A.

FIG. 8C shows a pressure-flow graph for three different tests of the embodiment shown in FIG. 8A.

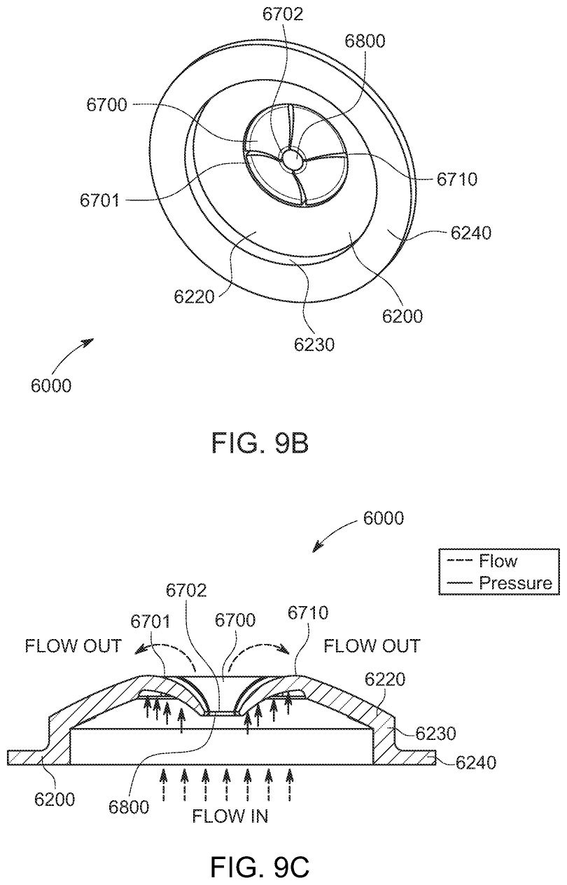

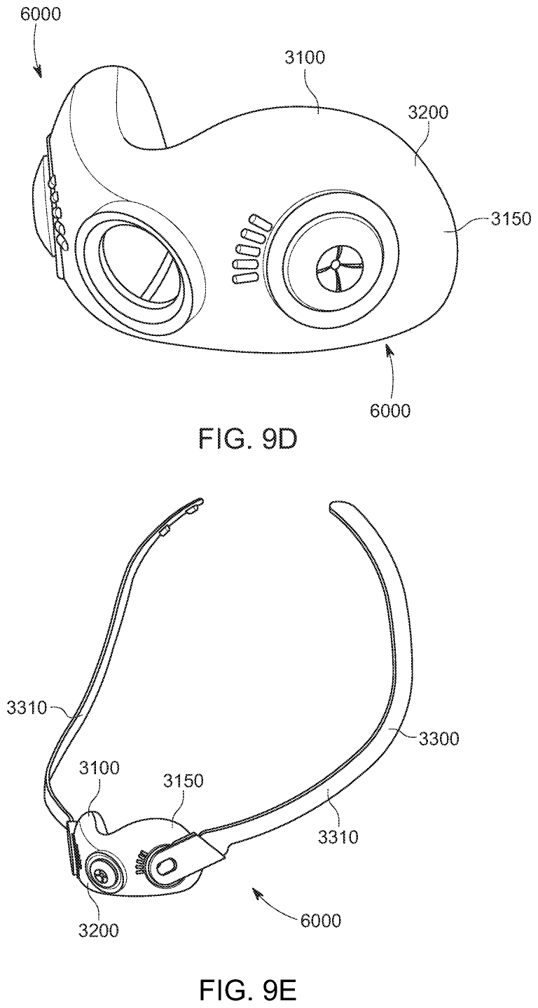

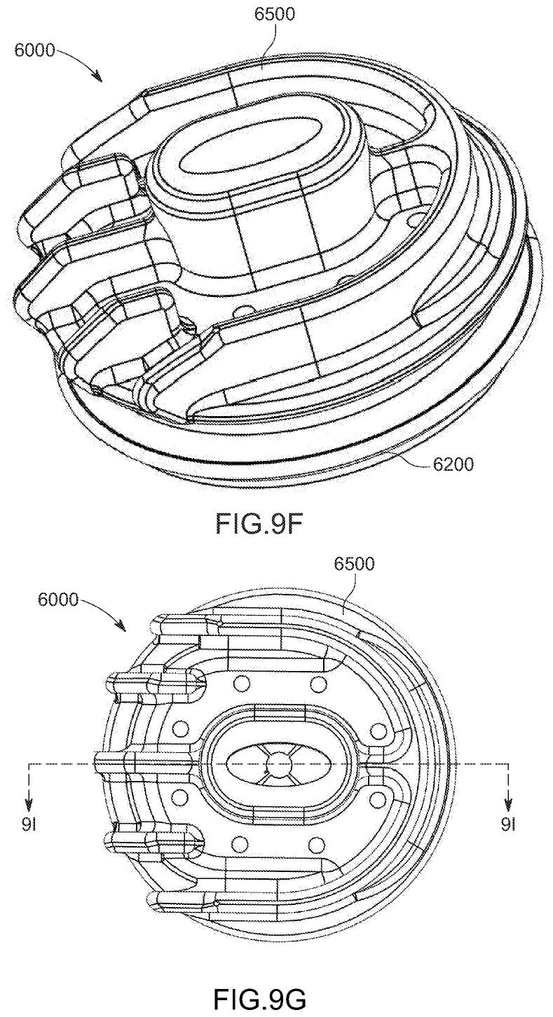

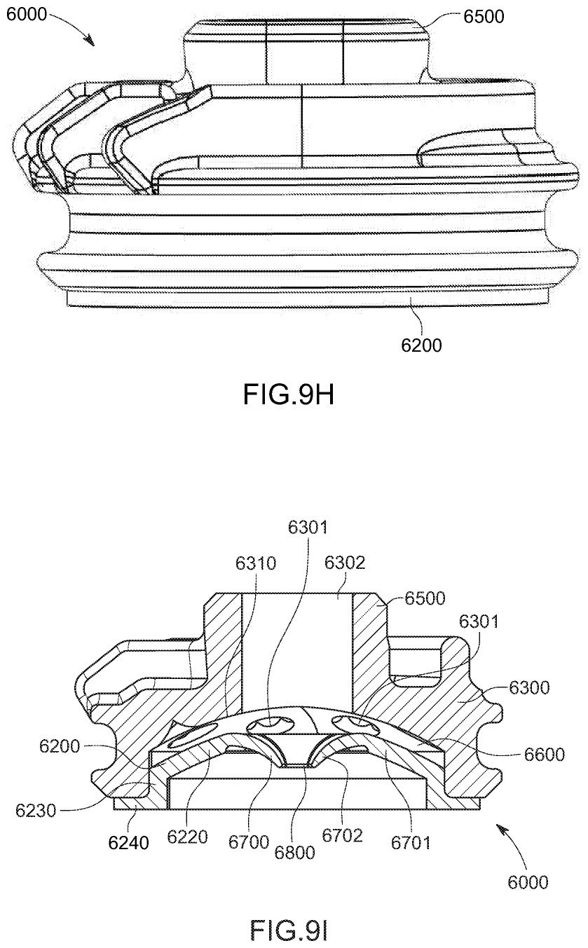

FIG. 9A shows a perspective view of a vent assembly according to another form of the present technology.

FIG. 9B shows a different perspective view of the vent assembly of FIG. 9A.

FIG. 9C shows a cross-sectional view of the vent assembly of FIG. 9A.

FIG. 9D shows a cushion module with a vent assembly of FIG. 9A.

FIG. 9E shows the cushion module with a vent assembly of FIG. 9A with a portion of a positioning and stabilising structure.

FIG. 9F shows a perspective view of a vent assembly according to another form of the present technology.

FIG. 9G shows a top view of the vent assembly of FIG. 9F.

FIG. 9H shows a side view of the vent assembly of FIG. 9F.

FIG. 9I shows a cross-sectional view along the plane A-A of FIG. 9G.

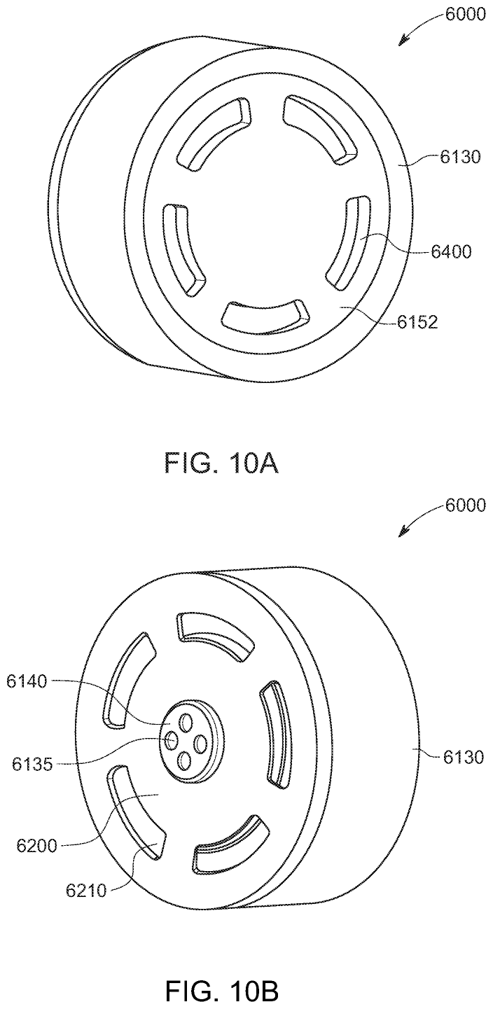

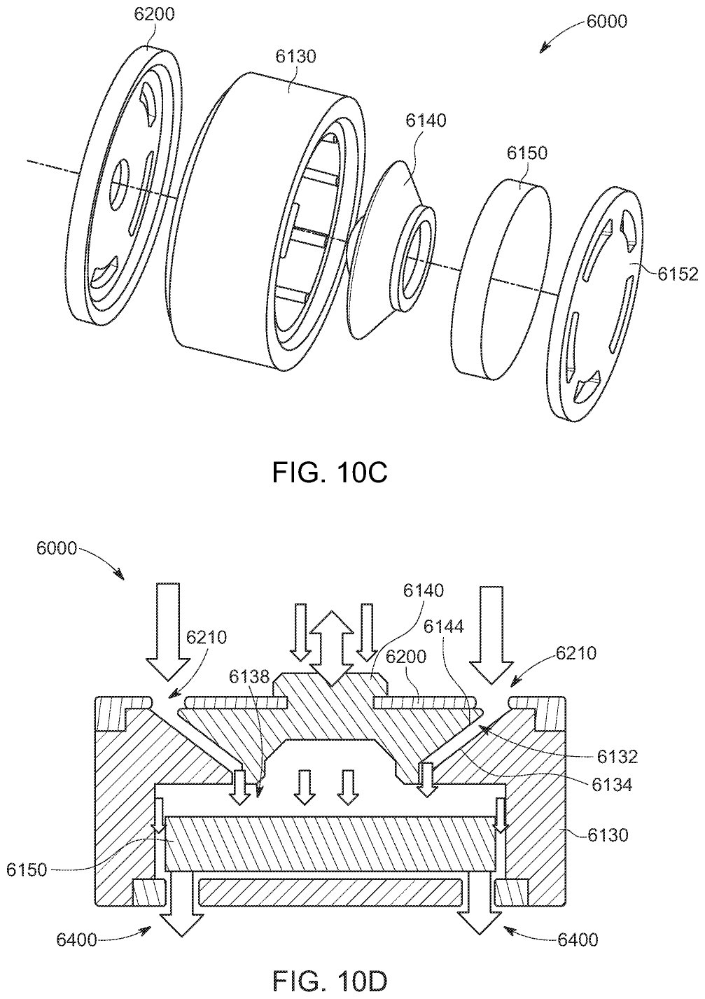

FIGS. 10A-10D show schematic illustrations of a vent assembly according to another example of the present technology.

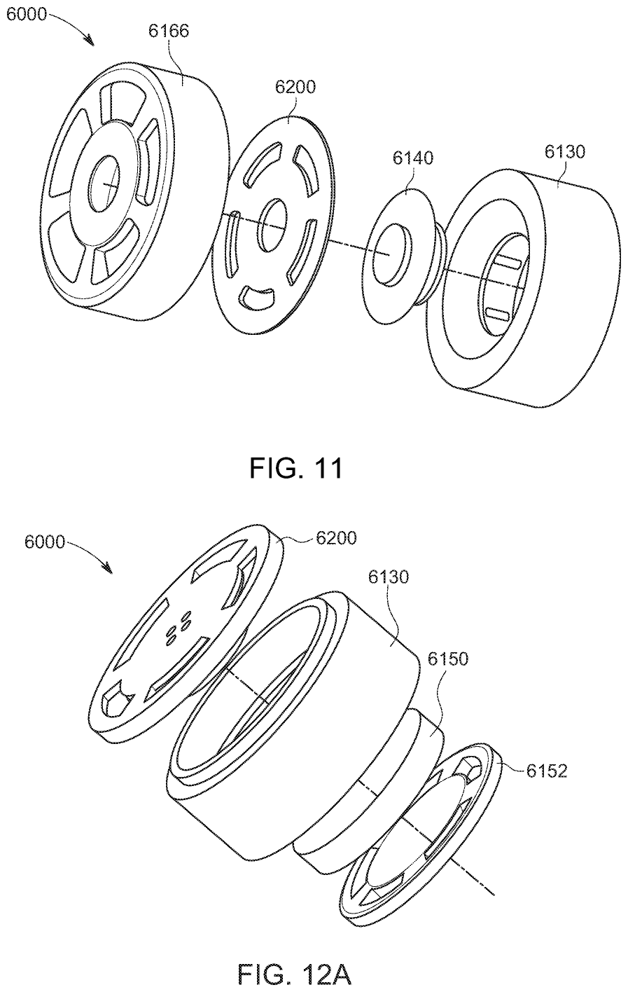

FIG. 11 shows a schematic illustration of a vent assembly according to another example of the present technology.

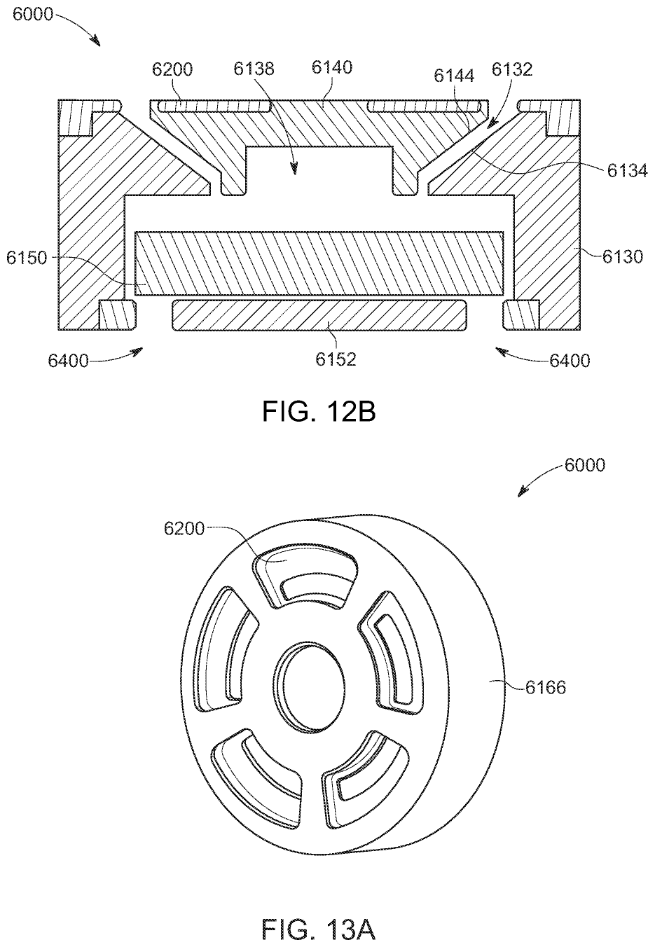

FIGS. 12A-12B show schematic illustrations of a vent assembly according to another example of the present technology.

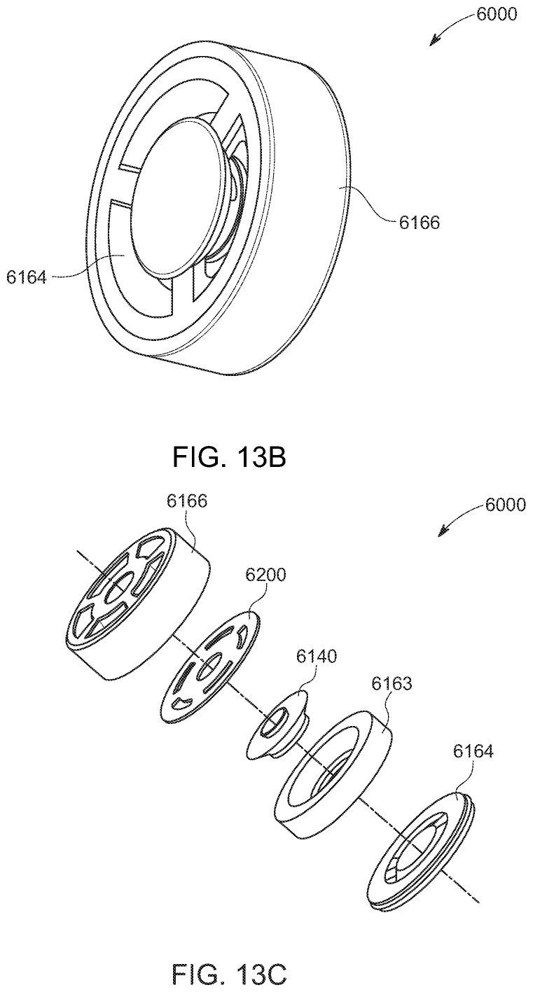

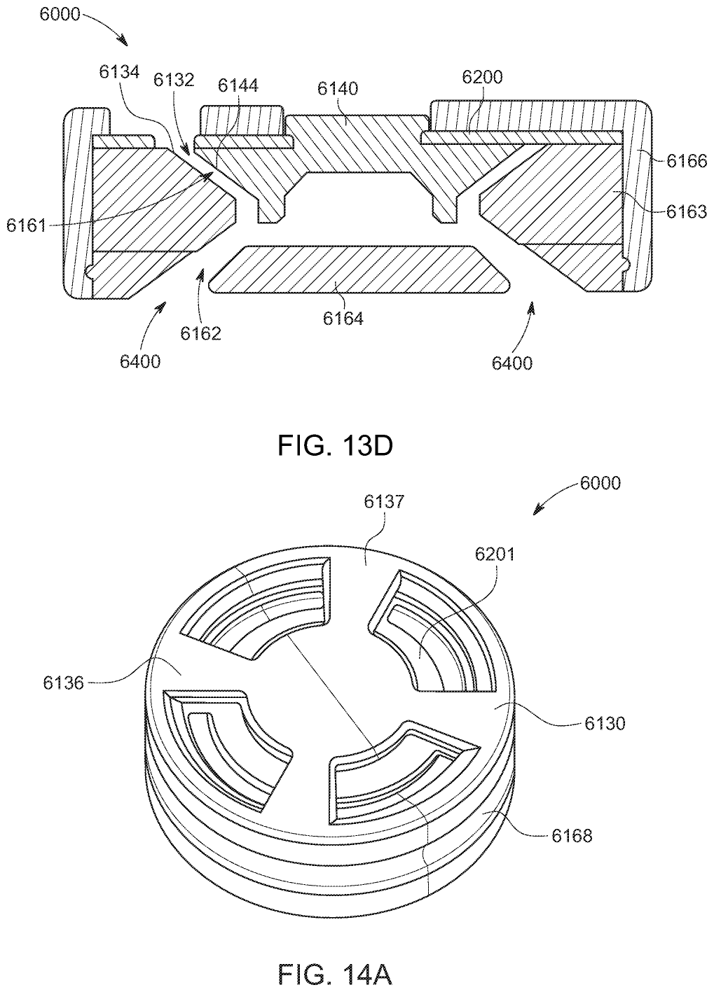

FIGS. 13A-13D show schematic illustrations of a vent assembly according to another example of the present technology.

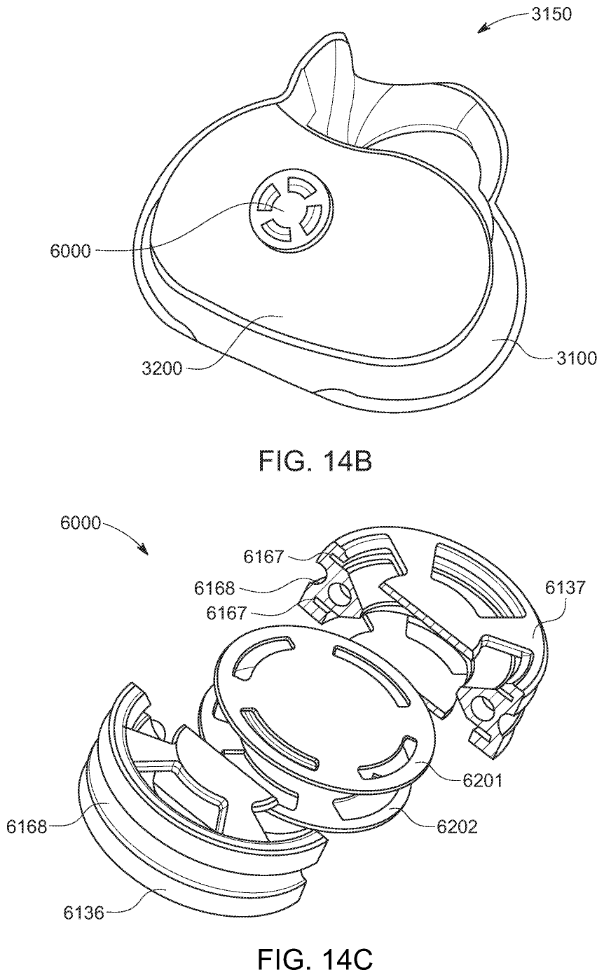

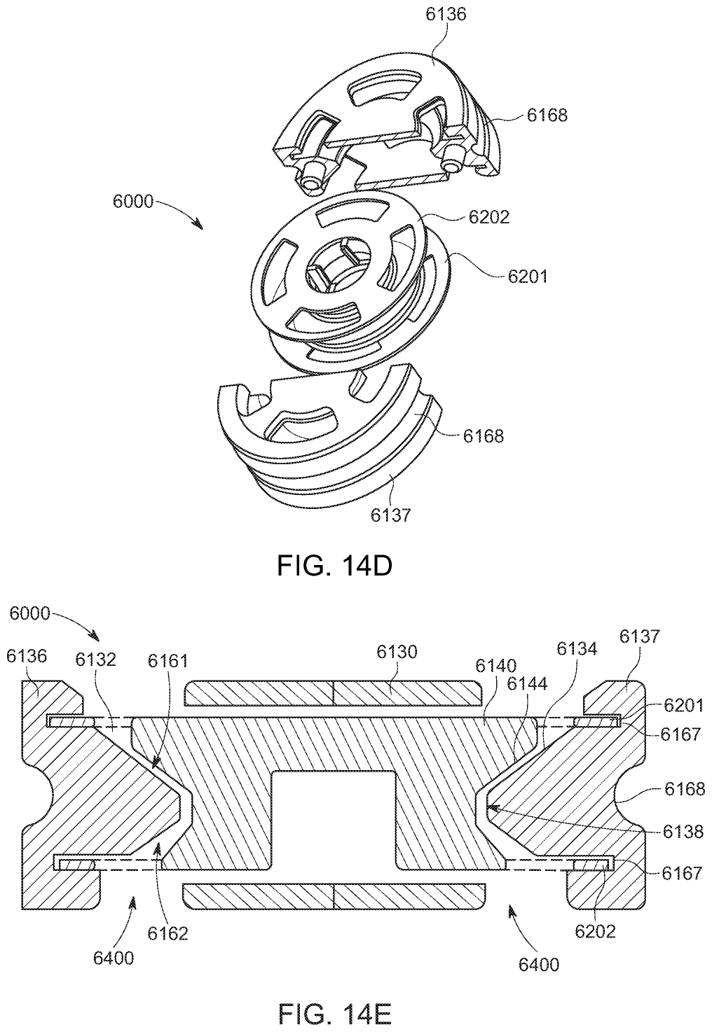

FIGS. 14A-14E show schematic illustrations of a vent assembly according to another example of the present technology.

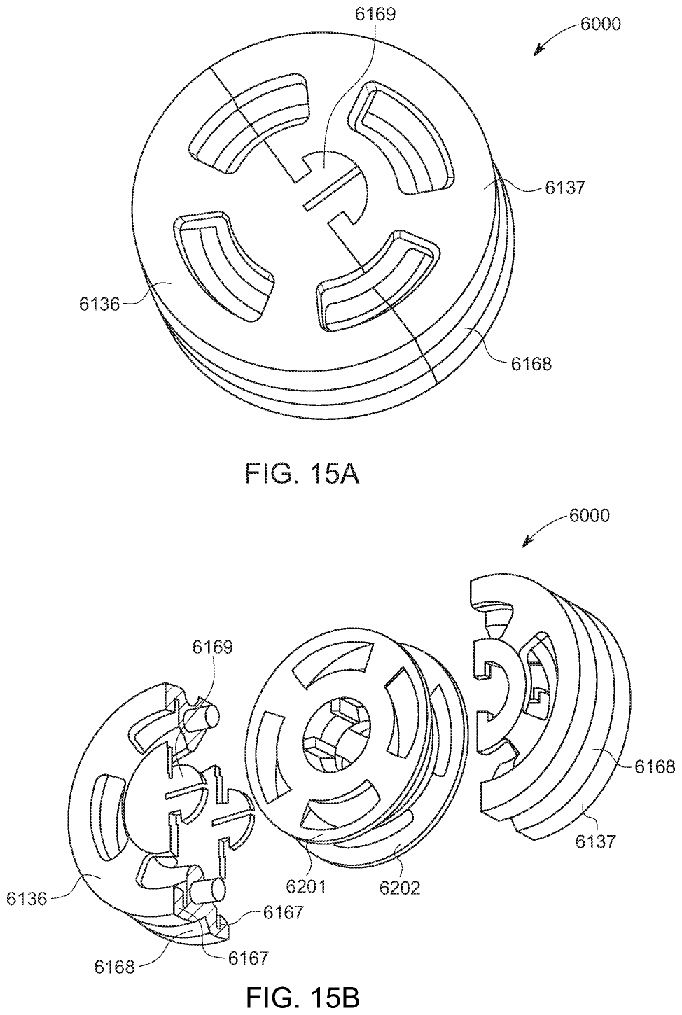

FIGS. 15A-15B show schematic illustrations of a vent assembly according to another example of the present technology.

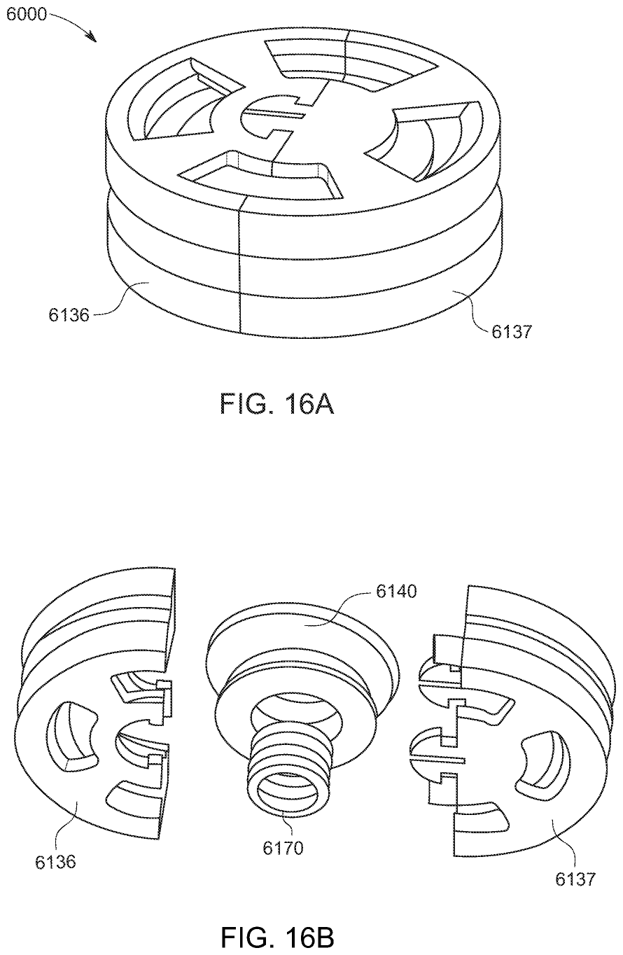

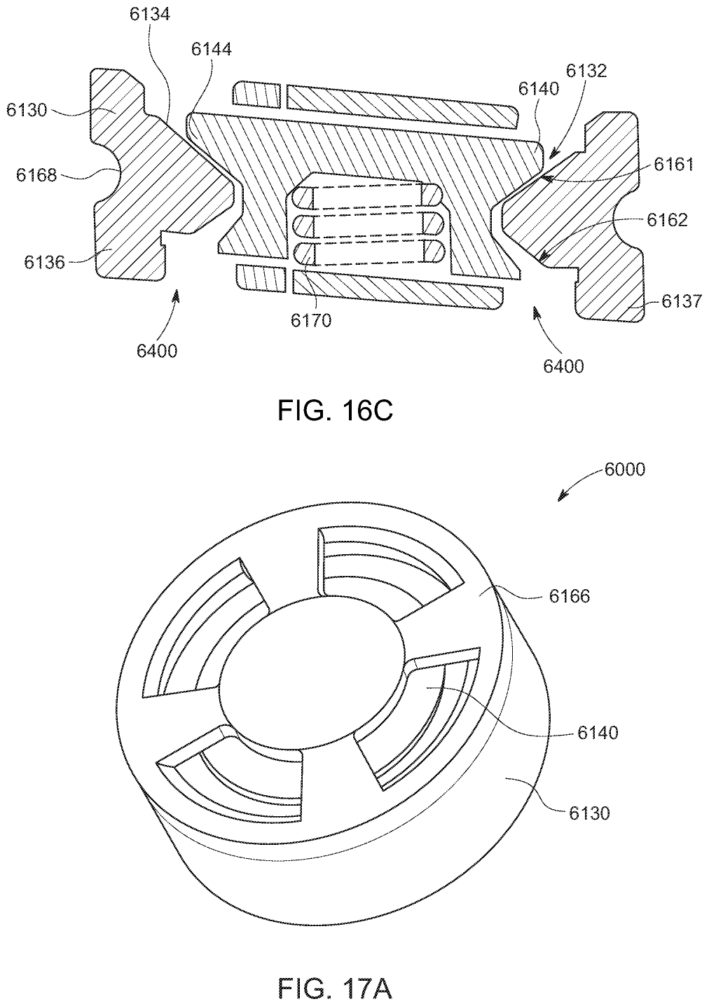

FIGS. 16A-16C show schematic illustrations of a vent assembly according to another example of the present technology.

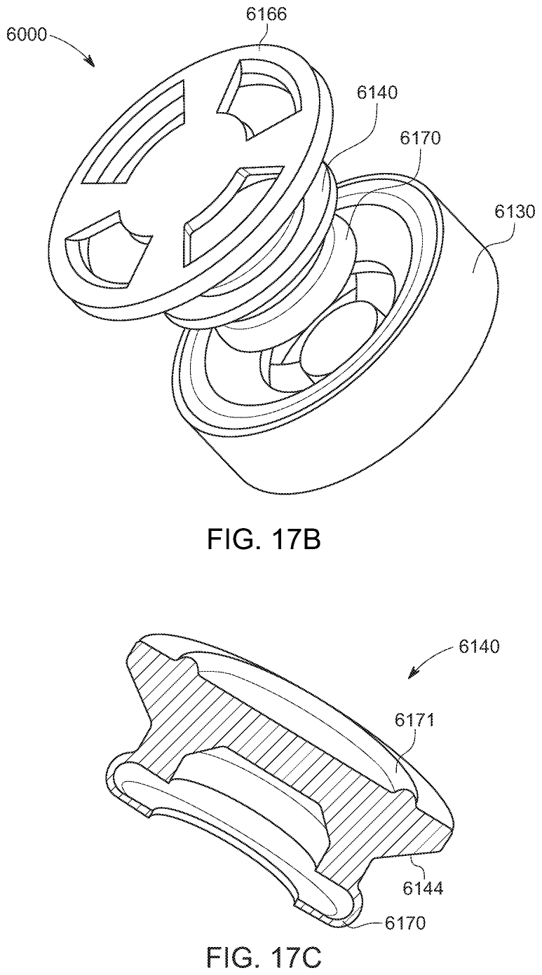

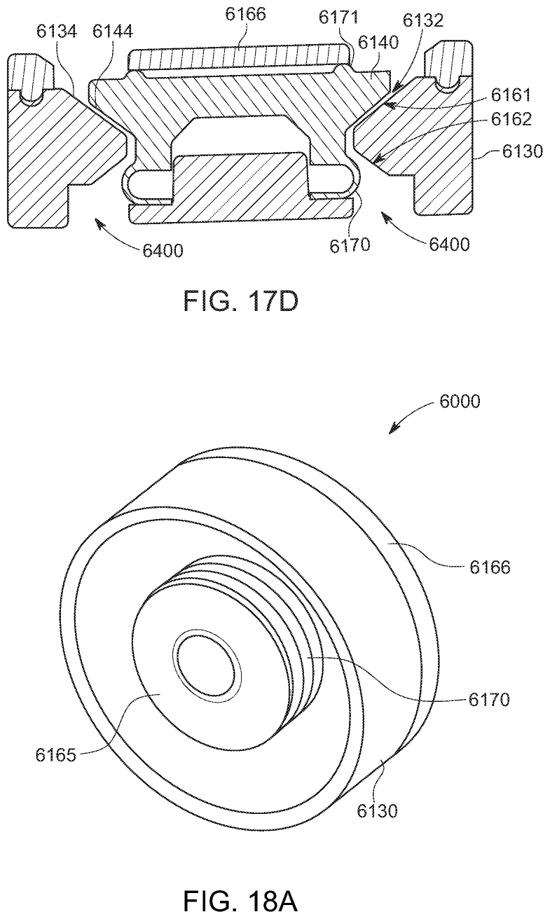

FIGS. 17A-17D show schematic illustrations of a vent assembly according to another example of the present technology.

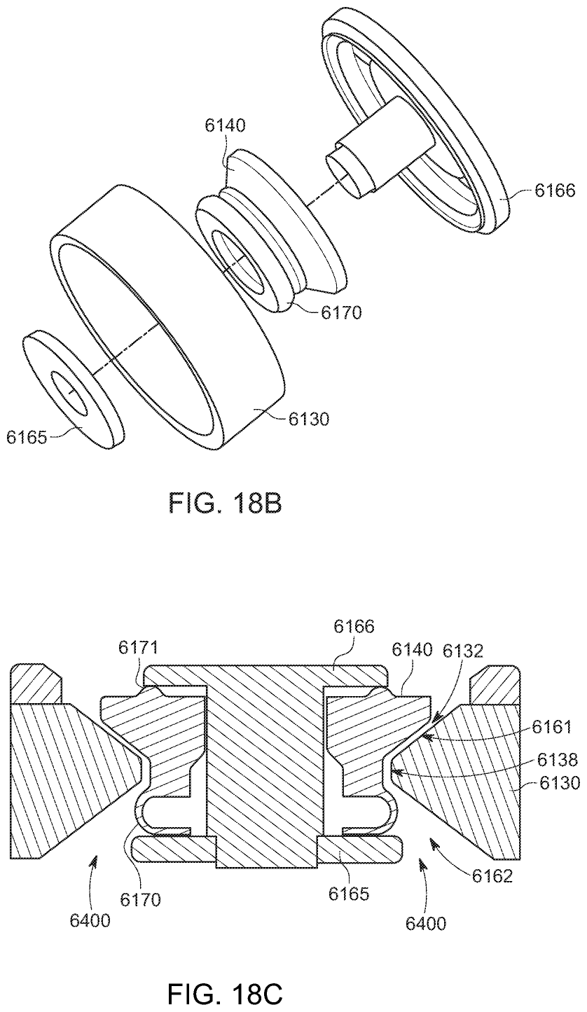

FIGS. 18A-18C show schematic illustrations of a vent assembly according to another example of the present technology.

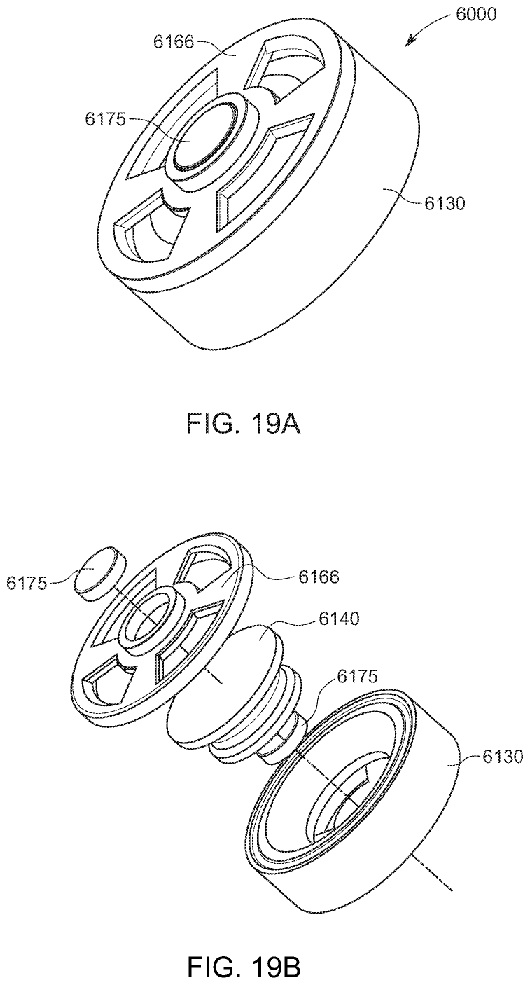

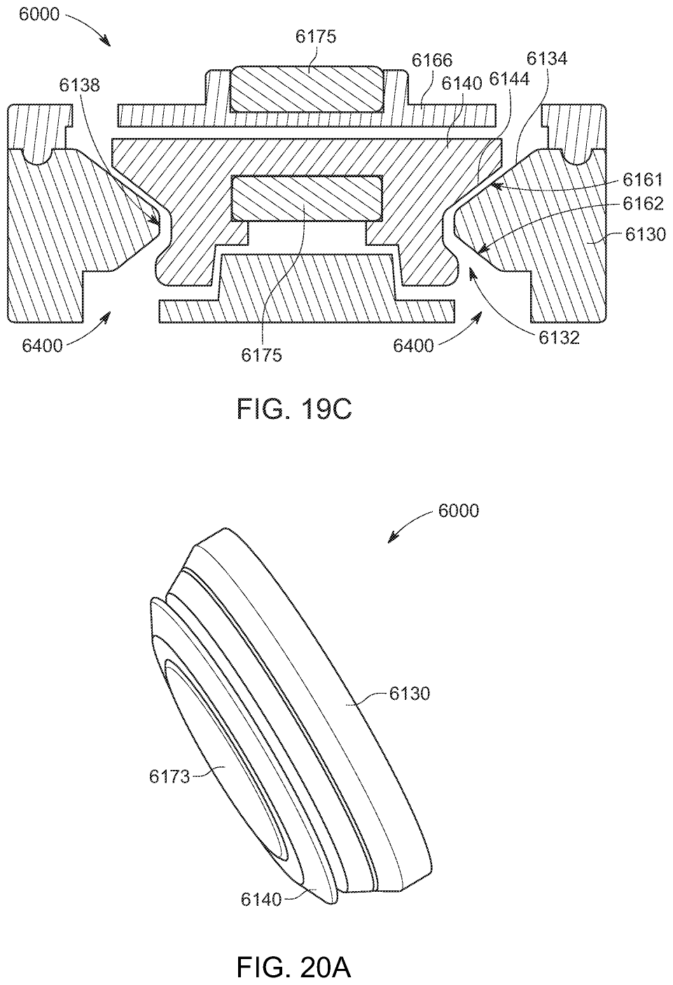

FIGS. 19A-19C show schematic illustrations of a vent assembly according to another example of the present technology.

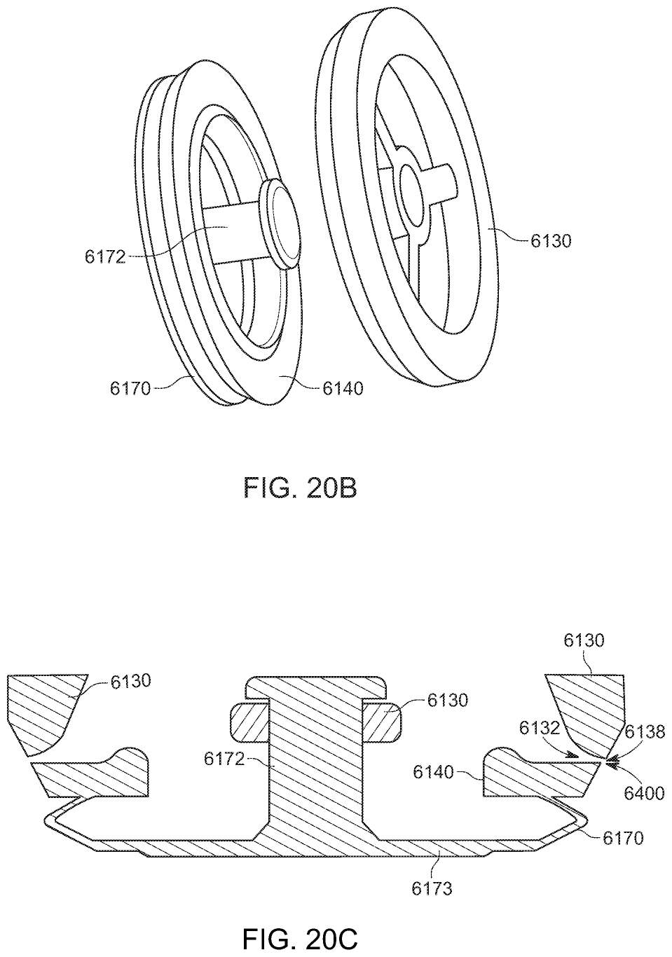

FIGS. 20A-20C show schematic illustrations of a vent assembly according to another example of the present technology.

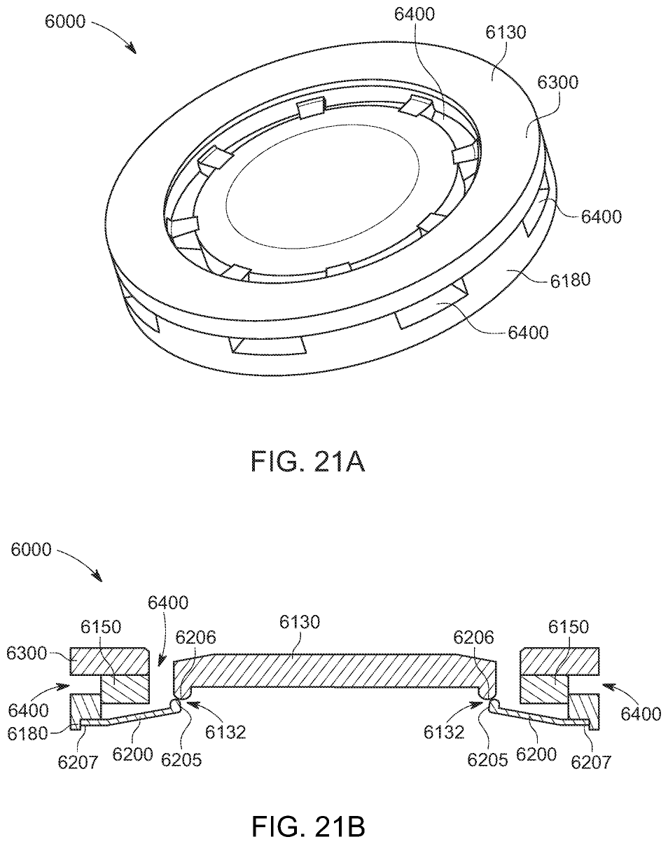

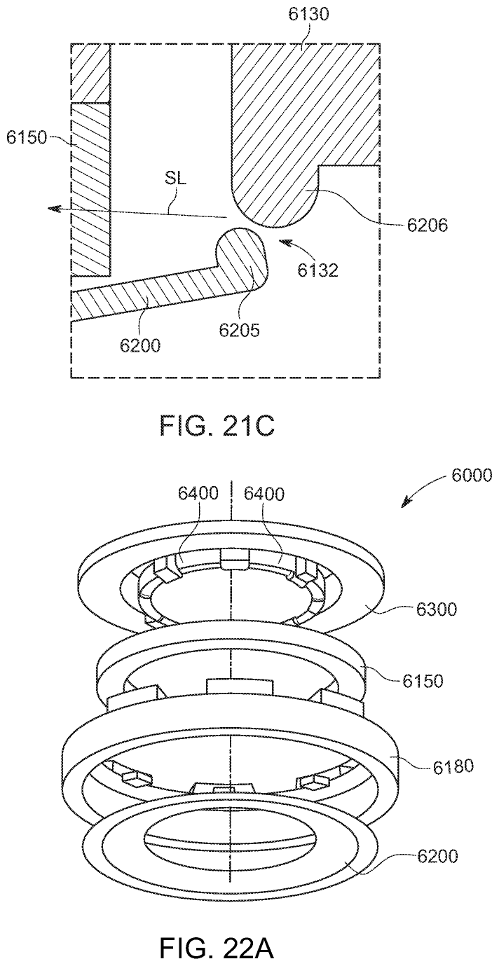

FIGS. 21A-21C show schematic illustrations of a vent assembly according to another example of the present technology.

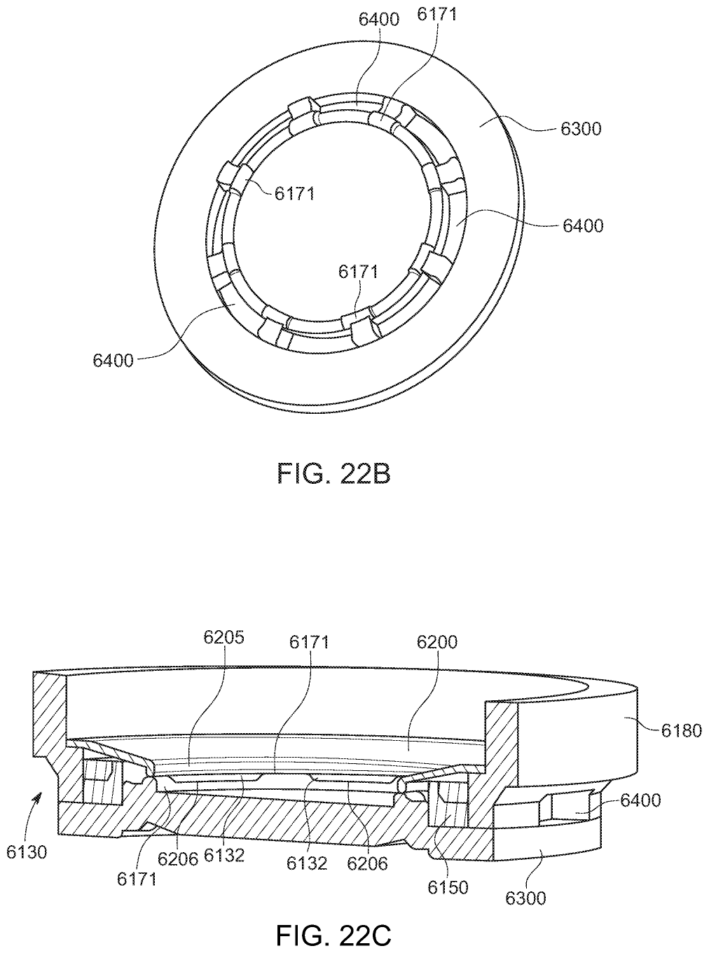

FIGS. 22A-22C show schematic illustrations of a vent assembly according to another example of the present technology.

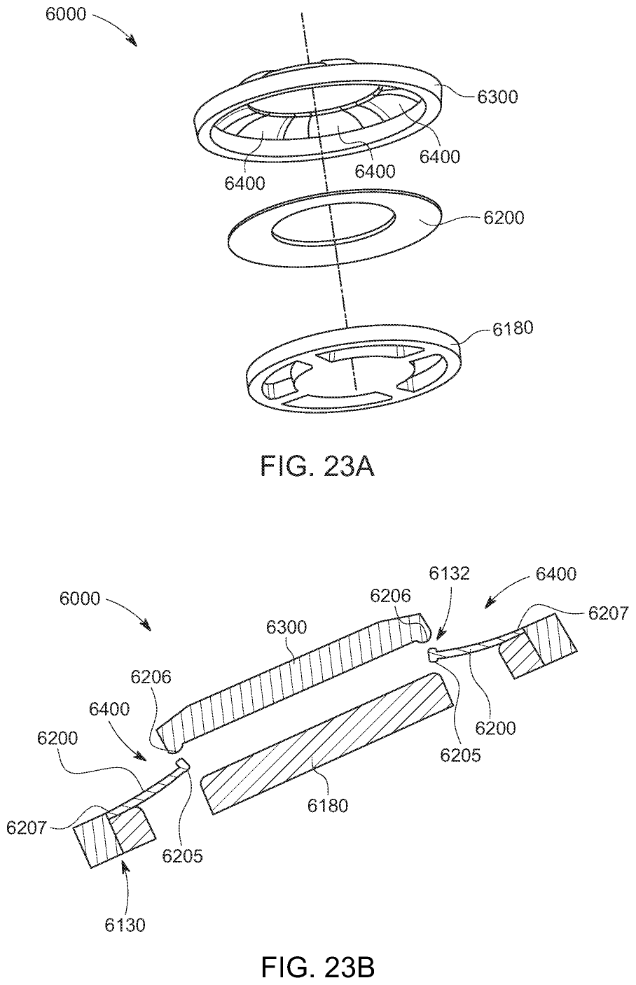

FIGS. 23A-23B show schematic illustrations of a vent assembly according to another example of the present technology.

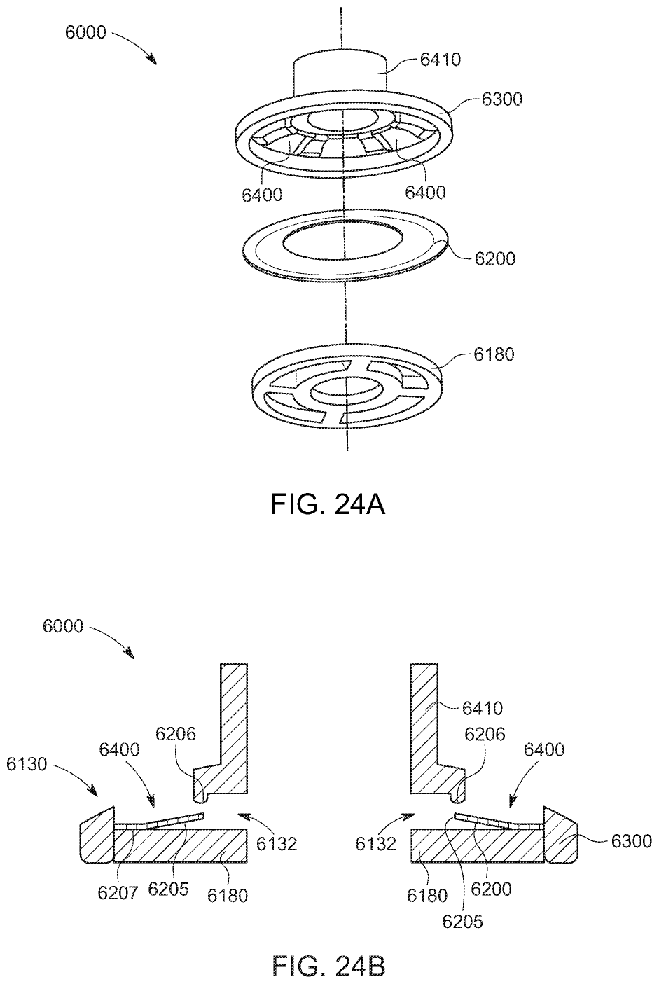

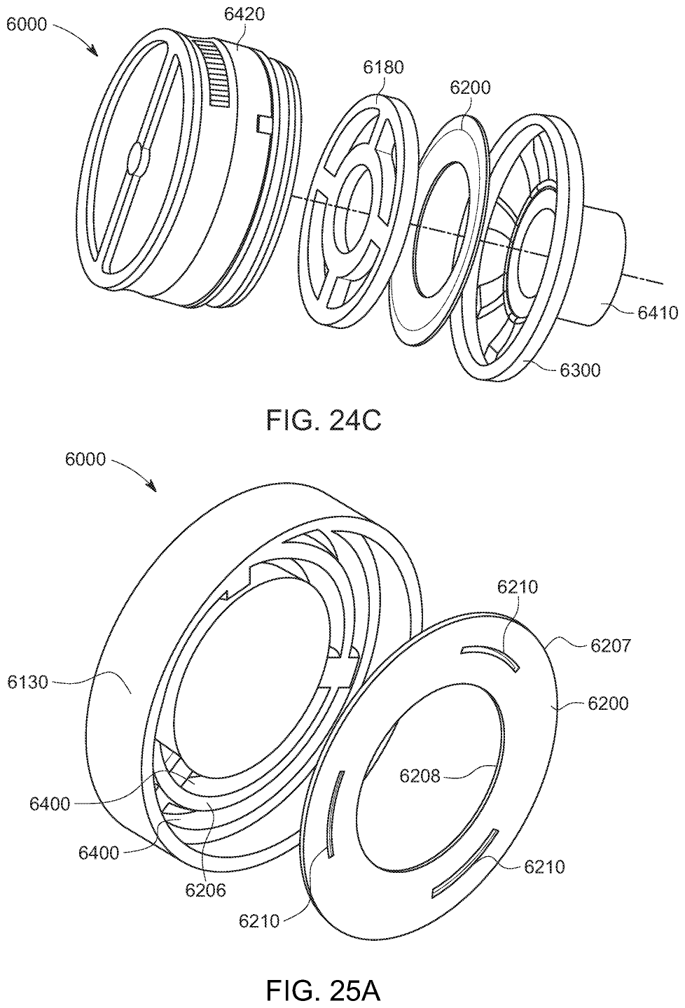

FIGS. 24A-24B show schematic illustrations of a vent assembly according to another example of the present technology.

FIG. 24C shows a schematic illustration of a vent assembly according to another example of the present technology.

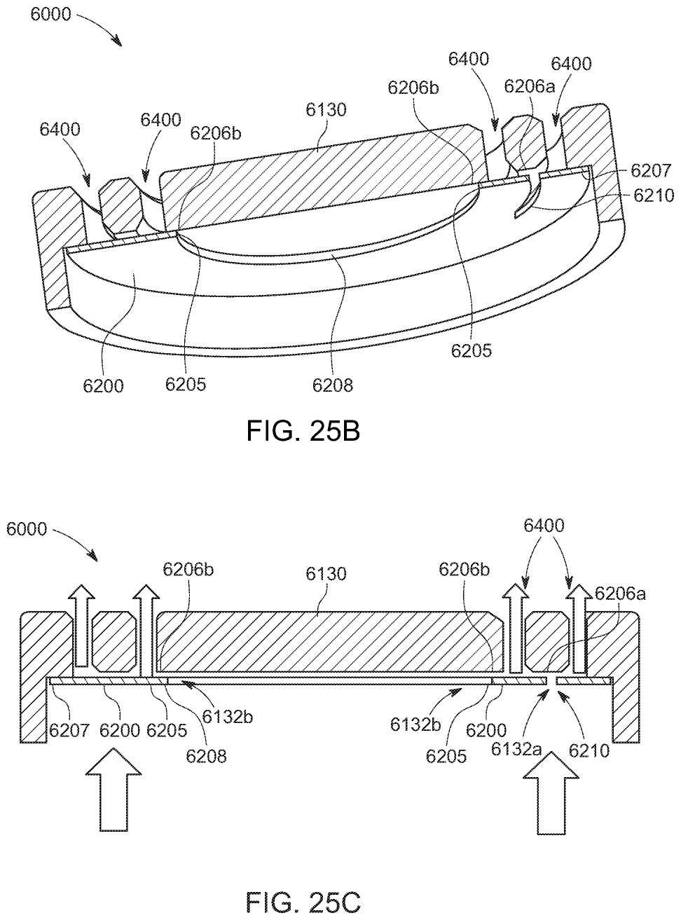

FIGS. 25A-25C show schematic illustrations of a vent assembly according to another example of the present technology.

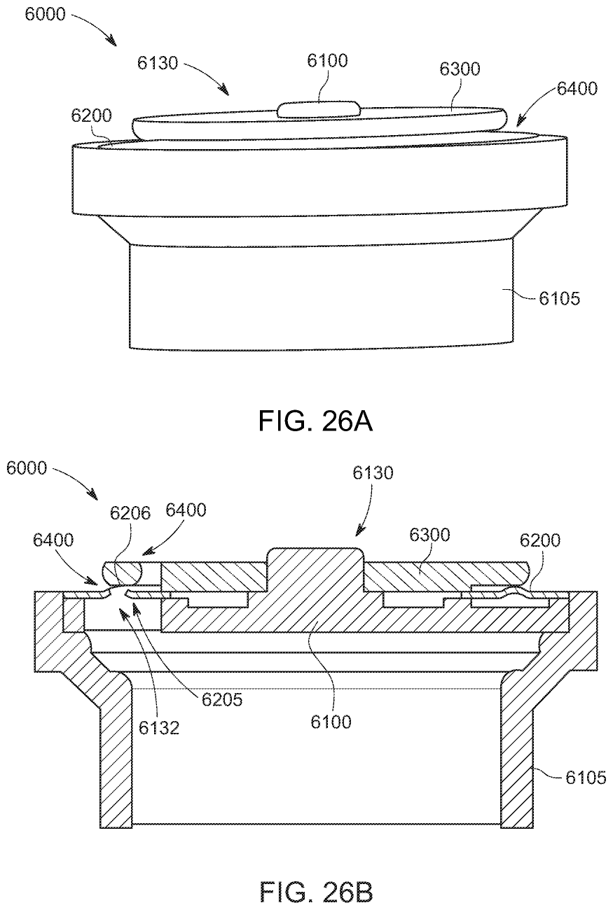

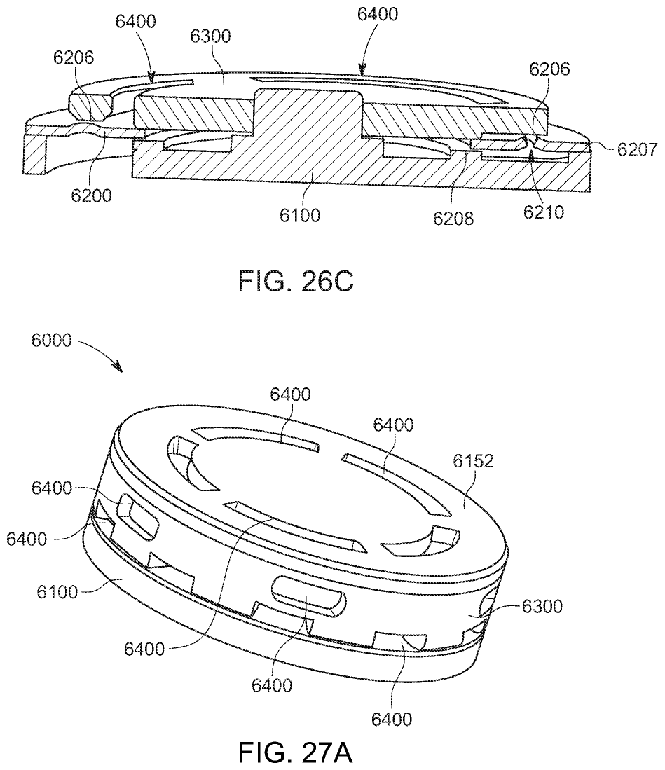

FIGS. 26A-26C show schematic illustrations of a vent assembly according to another example of the present technology.

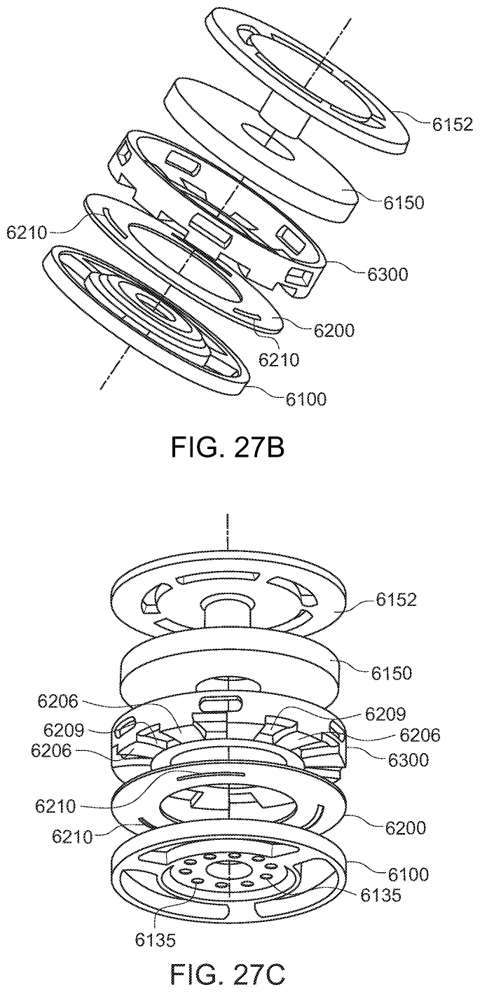

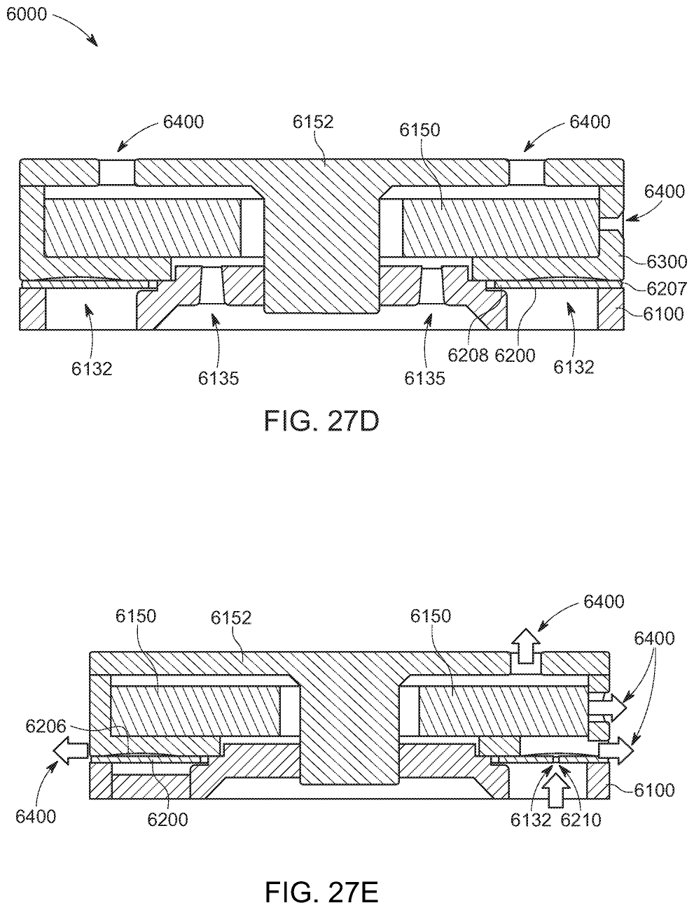

FIGS. 27A-27E show schematic illustrations of a vent assembly according to another example of the present technology.

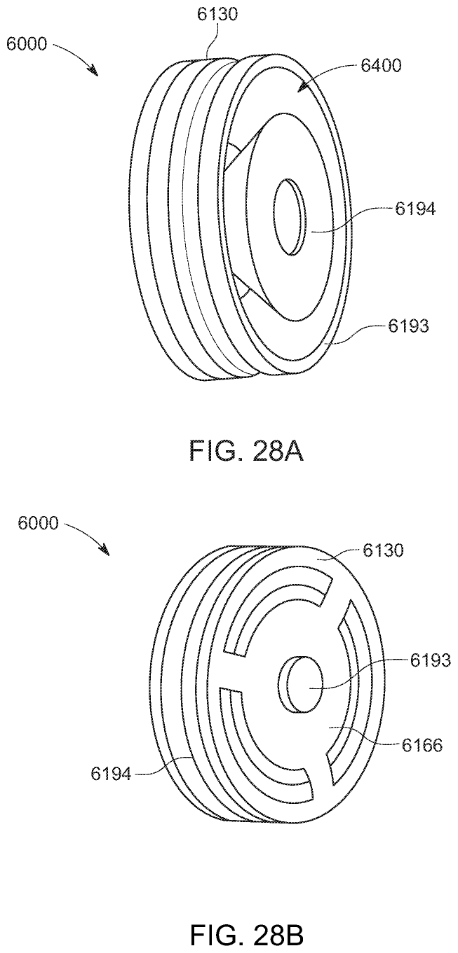

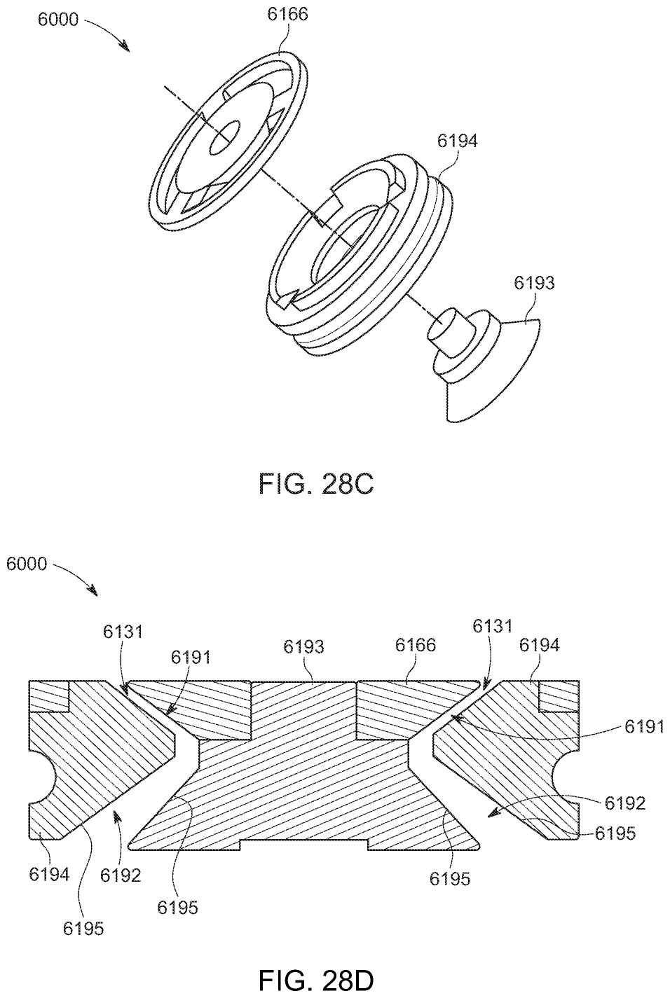

FIGS. 28A-28D show schematic illustrations of a vent assembly according to another example of the present technology.

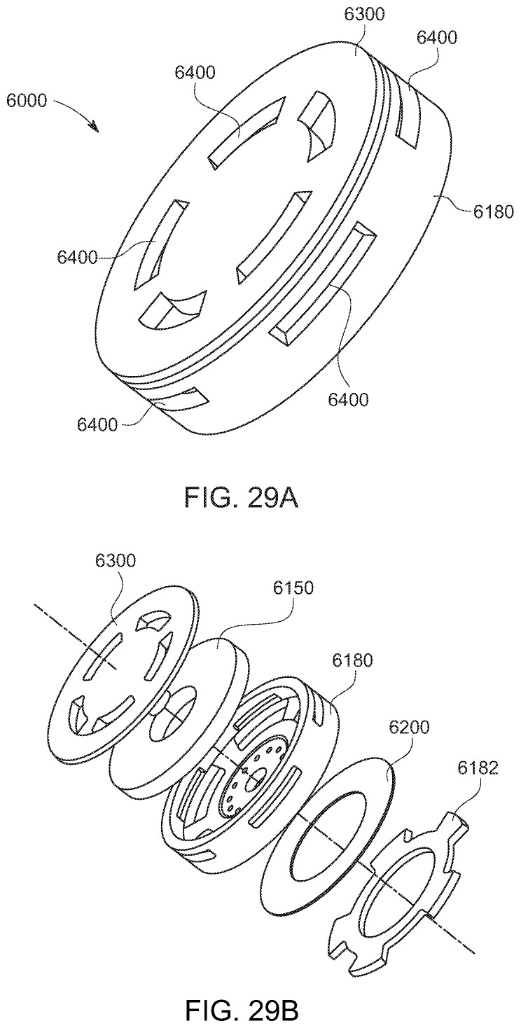

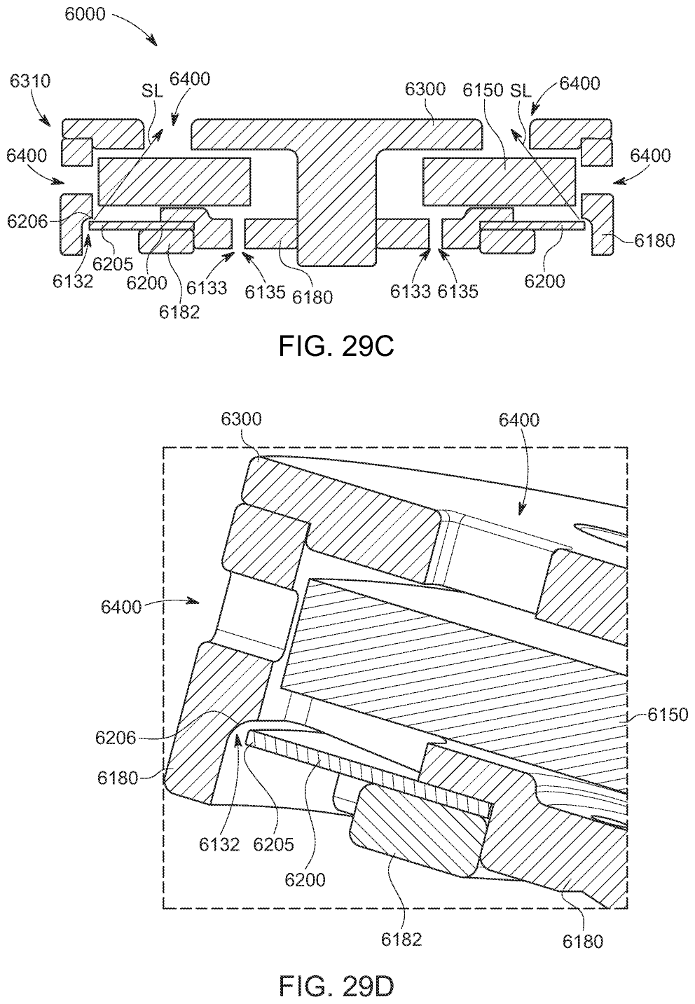

FIGS. 29A-29D show schematic illustrations of a vent assembly according to another example of the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3B, a non-invasive patient interface 3000 in accordance with another aspect of the present technology comprises the following functional aspects: a seal-forming structure 3000, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 4.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.1.7 Nasal Mask

In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice. This type of seal-forming structure 3100 may be referred to as a “nasal cushion” and a patient interface 3000 having such a seal-forming structure 3100 may be identified as a “nasal mask”.

### 4.3.1.8 Full Face Mask

In one form the patient interface 3000 comprises a seal-forming structure 3100 that forms a seal in use on a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “full face cushion” and the patient interface 3000 may be identified as a “full-face mask”.

### 4.3.1.9 Ultracompact Full-Face Mask

In one form the patient interface 3000 comprises a seal-forming structure 3100 that forms a seal in use on a patient's chin region (which may include the patient's lip inferior and/or a region directly inferior to the lip inferior), to an inferior and or anterior surface of the patient's pronasale and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may be known as an ultra-compact full face mask and may comprise an ultra-compact full face cushion.

### 4.3.1.10 Nasal Cradle Mask

In one form, for example as shown in FIG. 3B, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of the patient's pronasale and to the patient's nasal alae. The seal-forming structure 3100 may seal to the patient's lip superior. This type of seal-forming structure 3100 may be referred to as a “cradle cushion”, “nasal cradle cushion” or “under-the-nose cushion”, for example.

The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

### 4.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 4.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. Suitable for a small sized head, but not a large sized head.

### 4.3.3.1 Conduit Headgear

### 4.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3B, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth). This allows the conduit of air circuit 4170 providing the flow of pressurised air to connect to a connection port 3600 of the patient interface in a position other than in front of the patient's face, for example on top of the patient's head.

Since air can be contained and passed through headgear tubing in order to deliver pressurised air from the air circuit 4170 to the patient's airways, the positioning and stabilising structure 3300 may be described as being inflatable. It will be understood that an inflatable positioning and stabilising structure 3300 does not require all components of the positioning and stabilising structure 3300 to be inflatable. For example, in the example shown in FIG. 3B, the positioning and stabilising structure 3300 comprises the tubes 3350, which are inflatable, and the strap 3310, which is not inflatable.

In the form of the present technology illustrated in FIG. 3B, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes is compressed to block or partially block the Flow of gas along the tube, the other tube remains open to supply pressurised gas to the patient. In other examples of the technology, the patient interface 3000 may comprise a different number of tubes, for example one tube, or three or more tubes. In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head.

In the form of the technology shown in FIG. 3B the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used they may be indirectly connected together, for example each may be connected to a T-shaped connector having two arms/branches each fluidly connectable to a respective one of the tubes 3350 and a third arm or opening providing the connection port 3600 for fluid connection to the air circuit 4170 in use.

The tubes 3350 may be formed from a flexible material, such as an elastomer, e.g. silicone or TPE, or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

As described in U.S. Pat. No. 6,044,844, the contents of which are incorporated herein, the tubes 3350 may be crush resistant to avoid the flow of breathable gas through the tubes being blocked if either is crushed during use, for example if it is squashed between a patient's head and pillow. Crush resistant tubes may not be necessary in all cases as the pressurised gas in the tubes may act as a splint to prevent or at least restrict crushing of the tubes 3350 during use. A crush resistant tube may be advantageous where only a single tube 3350 is present as if the single tube becomes blocked during use the flow of gas would be restricted and therapy will stop or reduce in efficacy. In some examples, the tubes 3350 may be sized such that each tube 3350 is able to provide sufficient flow of gas to the plenum chamber 3200 on its own should one of the tubes 3350 become blocked.

Each tube 3350 may be configured to receive a flow of air from the connection port 3600 on top of the patient's head and to deliver the flow of air to the seal-forming structure 3100 at the entrance of the patient's airways. In the example shown in FIG. 3B, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force vector in a posterior and superior direction (e.g. a posterosuperior direction). A sealing force vector with a posterosuperior direction may facilitate the seal-forming structure 3100 forming a good seal to both the inferior periphery of the patient's nose and the anterior-facing surfaces of the patient's face on either side of the patient's nose and the patient's lip superior.

### 4.3.3.1.2 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3B, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

### 4.3.3.1.3 Conduit Headgear Straps

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3B, the patient interface 3000 comprises a strap 3310 forming part of the positioning and stabilising structure 3300. The strap 3310 may be known as a back strap or a rear headgear strap, for example. In other examples of the present technology, one or more further straps may be provided. For example, patient interfaces 3000 according to examples of the present technology having a full face cushion may have a second, lower, strap configured to lie against the patient's head proximate the patient's neck and/or against posterior surfaces of the patient's neck.

In the example shown in FIG. 3B, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 3B, the positioning and stabilising structure 3300 comprises a pair of tabs 3320. In use a strap 3310 may be connected between the tabs 3320, The strap 3310 may be sufficiently flexible to pass around the back of the patient's head and lie comfortably against the patient's head, even when under tension in use.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

In certain aspects of the present technology, a vent assembly 6000 is provided that is configured to form or provide a vent 3400 for the respiratory therapy system in use.

### 4.3.4.1 Location of the Vent Assembly

As is the case with the forms of the technology shown in FIGS. 6A to 6E, 9D and 9E, the vent assembly 6000 may form part of the plenum chamber 3200. For example, the vent assembly 6000, as part of the plenum chamber 3200, may enclose a volume of space containing pressurised air during use of the patient interface 3000. In some forms, for example as shown in FIGS. 6E and 9D, the vent assembly 6000 and another component, for example a cushion module 3150, together form the plenum chamber 3200. The cushion module 3150 may also comprise the seal-forming structure 3100. In other forms, the plenum chamber 3200 may be formed by a frame, or a combination of a frame and a cushion module 3150. In such forms, the vent assembly 6000 may be provided to the frame, the cushion module 3150, or both. In the form shown in FIG. 9D, there are two vent assemblies 6000 provided on each of the left side and right side of the cushion module 3150. In FIG. 9E, the vent assembly is located at a central region of the cushion module 3150.

In some forms of the technology, the cushion module 3150 may be provided with a plurality of vent assemblies 6000. The plurality of vent assemblies 6000 may be arranged in a line, in a circular arrangement or along a plurality of lines, along one or more parts of the cushion module 3150, for example.

In one form of the present technology, the patient interface 3000 is configured so that, in use, the vent assembly 3400 is located proximate the airway entrance of a patient, for example proximate the nasal entrance as in the case of the patient interface 3000 of FIGS. 6E. 9D and 9E. The seal-forming structure 3100, constructed and arranged to form a seal with a region of the patient's face surrounding the airway entrance, includes an aperture therein. The aperture is fluidly connected to the vent assembly 6000, such that a flow of exhaled gas from the airway of the patient passes through the aperture of the seal-forming structure 3100 and may flow to the vent assembly 6000.

It has been explained above that, in certain forms of the technology, for example the form of the technology shown in FIG. 3B, the positioning and stabilising structure 3300 comprises one or more tubes 3350 that deliver pressurised air received from a conduit forming part of the air circuit 4170 from the RPT device to the patient's airways. In some forms, the vent assembly 6000 may be further located on the gas delivery tubes 3350 of such conduit headgear. In such forms, the vent assembly 6000 may be located proximate an end of the tubes 3350 that fluidly connect to the plenum chamber 3200.

In some forms of the technology, the vent assembly 6000 may be located in the air circuit 4170 or in a decoupling structure, e.g., a swivel or elbow.

### 4.3.4.2 Vent Assembly Overview

The vent assembly 6000 for a respiratory pressure therapy system may be configured in use to convey a vent flow of gases exhaled by a patient from a first volume, which is interior to the respiratory pressure therapy system, to ambient. The first volume may be supplied with a pressurised flow of gas from the RPT device 4000 in use, and also may be a volume that receives exhaled gas from the patient. The first volume may depend on the location of the vent assembly 6000 within the respiratory pressure therapy system. For instance, in forms of the technology where the vent assembly 6000 is formed as part of the plenum chamber 3200, the first volume may be or at least include the volume inside the plenum chamber 3200. In other forms, the first volume may be formed, at least in part, by a volume inside, for example headgear tubes 3350, air circuit 4170, a decoupling structure or elbow, in addition to the volume inside the plenum chamber 3200.

In one form of the present technology, the vent assembly 6000 may comprise a vent base 6100 having formed therein a vent base aperture 6110. The vent assembly 6000 may further comprise a flexible membrane 6200 mounted within the vent assembly 6000, for example to the vent base 6100, and spanning across the vent base aperture 6110. The membrane 6200 may have formed therein a membrane aperture 6210 to allow the vent flow to pass through the membrane 6200. The vent assembly 6000 may also comprise a vent cap 6300 connected to the vent base 6100, for example mounted directly to the vent base 6100 or connected to it via one or more intermediate components. The vent cap 6300 may be located downstream of the membrane 6200 relative to the vent flow. The vent cap 6300 may be positioned in the path of the vent flow through the membrane aperture 6210.

FIGS. 6A to 6E show a vent assembly 6000 according to one form of the technology. The vent assembly 6000 has a generally cylindrical geometry with the vent base 6100, membrane 6200, and vent cap 6300 all having a circular cross-sectional shape with aligned longitudinal axes. In other forms of the technology, the vent assembly 6000 may have a different geometrical layout for instance a rectangular cuboid or truncated pyramid. The vent base 6100, membrane 6200, and vent cap 6300 may have a corresponding cross-sectional shape to that of the vent assembly 6000 or one or more components may have a different shape.

FIGS. 7A to 7F show a vent assembly 6000 according to another form of the technology. The vent base 6100 is not shown in the figures but may comprise a component of the respiratory pressure therapy system, for instance one of the plenum chamber 3200, seal-forming structure 3100, air circuit 4170, or decoupling structure. The membrane 6200 and the vent cap 6300 are mounted around their periphery directly to the vent base 6100 in this particular example. In other examples the membrane 6200 and or vent cap 6300 may be connected to the vent base 6100 via one or more intermediate components.

In the case of both the vent assembly 3400 shown in FIGS. 6A to 6E and FIGS. 7A to 7F, in use, the pressure of the gas in the first volume acts on the membrane 6200 such that changes in the pressure of the gas in the first volume causes the membrane 6200 to flex. The varying flex in the membrane 6200 varies a position of the membrane 6200 or at least a portion thereof relative to the vent cap 6300. As will be described in more detail later, this mechanism may be used to control the vent flow through one or more vent outlets 6400 to ambient, e.g. the volumetric rate of vent flow (e.g. in L/min). Generally, when the pressure of the gas in the first volume increases the membrane 6200 flexes into a position closer to the vent cap 6300 and there is a reduced vent flow through the vent outlet(s) 6400.

A patient interface 3000 may require a vent flow rate that is at least large enough to “wash out” exhalate from the plenum chamber 3200 (to prevent rebreathing of exhaled air and the associated carbon dioxide) for the lowest therapy pressure at which a patient interface 3000 may be used. This requirement may be based on the size of the plenum chamber 3200. In a vent with fixed size orifice(s) and no moving parts, when the pressure of the air entering the first volume of the vent assembly 6000 is higher, as may be required by some patients for appropriate treatment, more air is forced to exit the vent outlet(s) 6400 to ambient such that at higher treatment pressures, more air is typically lost by the system due to an increased vent flow rate. The respiratory pressure therapy device generating the flow of air needs to compensate for this loss, resulting in increased power consumption. Furthermore, the higher vent flow rates may be noisy or uncomfortable to the patient or a bed partner. In one form of the present technology, the configuration (e.g. shape, size, orientation and/or position) adopted by the membrane 6200 is based on the pressure of gas in the first volume. A higher regulated air pressure entering the first volume will exert more force on the membrane 6200, urging the membrane closer to the vent cap 6300, such that the vent outlet(s) 6400 are occluded to a greater extent, reducing what would otherwise have been an increased flow rate through the vent outlet(s) 6400 to ambient.

The way in which the configuration of the membrane 6200 varies with changes in pressure, and consequently the way in which the vent flow rate changes with pressure, may be determined based on certain characteristics of the membrane 6200. Examples of such characteristics and how they may be varied will be discussed further below. In certain forms, the vent assembly 6000 may be configured so that, in use, the vent flow rate of exhaled air from the first volume through the vent assembly 6000 to ambient is substantially constant for a range of pressures inside the first volume. In some forms the vent assembly 6000 is configured such that in use the vent flow rate from the first volume increases with increasing pressure to a lesser extent than it would increase in a fixed-size aperture vent.

In the example shown in FIGS. 9A-9E, the vent assembly 6000 comprises a plurality of flaps 6700. The plurality of flaps 6700 may form a central aperture 6800, through which a flow of air can pass in use from the first volume to surrounding ambient air. In some forms, the plurality of flaps 6700 may connect directly to a vent base 6100 or membrane 6200, as described below. Alternatively, the plurality of flaps may connect directly to a part of the cushion module 3150, elbow or air circuit 4120, i.e. there may be no membrane 6200 present in the vent assembly. In some forms, the plurality of flaps 6700 may be located at a central region of the membrane 6200. The central aperture 6800 of the plurality of flaps 6700 may form the aperture in the central region of the membrane.

In certain forms of the present technology, since the plurality of flaps 6700 are in contact with the first volume, the pressurised air in the first volume is able to act on the flaps 6700 and cause them to move. The movement of the flaps 6700 may change the area of the central aperture 6800. Consequently, the configuration adopted by the plurality of flaps 6700 is based on the pressure of gas in the first volume. The central aperture 6800 may in some forms be the vent outlet to ambient, such that changing the area of the central aperture 6800 changes the flow rate through the vent outlet to ambient.

The plurality of flaps 6700, along with the membrane 6200 in forms of the technology comprising a membrane 6200, may be configured such that, in use when the pressure inside the first volume increases, the plurality of flaps 6700 move to a first configuration and when the pressure inside the first volume decreases, the plurality of flaps move to a second configuration. An area of the central aperture 6800 is greater in the second configuration compared to the first configuration. The movement of the flaps 6700 may be movement relative to the membrane 6200. Alternatively, in some forms, the membrane 6200 may move, at least in part, with the flaps 6700.

The way in which the configuration of the plurality of flaps 6700 varies with changes in pressure, and consequently the way in which the vent flow rate changes with pressure, may be determined based on certain characteristics of the plurality of flaps 6700. Examples of such characteristics and how they may be varied will be discussed further below. In certain forms, the vent assembly 6000 may be configured so that, in use, the vent flow rate of exhaled air from the first volume through the vent assembly 6000 to ambient is substantially constant for a range of pressures inside the first volume. Operation of the vent assembly 6000 and the configuration of the plurality of flaps 6700 is discussed further below.

### 4.3.4.3 Vent Membrane

The membrane 6200 may be constructed and arranged to allow the regulation of a vent flow of exhaled gas from the airway of a patient leaving the vent assembly 6000 to ambient.

In the example of the technology shown in FIGS. 6A-6F, the membrane 6200 is mounted to the vent base 6100. For example, the membrane 6200 may be sealingly mounted to the vent base 6100. The membrane 6200 may be mounted to the vent base 6100 around a periphery of the membrane 6200. The membrane 6200 may be mounted to the vent base 6100 via any appropriate mounting mechanism, for example one of a clip mechanism, friction fit, snap-fit connection, or glue/adhesive. The membrane 6200 may alternatively be mounted within the vent assembly 6000 to another component in other forms of the present technology.

In certain forms, the membrane 6200 spans across the vent base aperture 6110. The membrane 6200 may be positioned such that it bridges the vent base aperture 6110, for instance the periphery of the membrane 6200 may be mounted to the vent base 6100 around the vent base aperture 6110 such that the central region of the membrane 6200 is positioned over the vent base aperture 6110. The membrane 6200 may therefore at least partially cover the vent base aperture 6110 such that it at least partially blocks or obstructs the flow path of the vent flow of gas from the vent base aperture 6110.

In the forms of the technology illustrated, the membrane 6200 is formed to be flexible such that it can flex or bend as the pressure in the first volume increases and decreases. For example, the membrane 6200 may be formed from a flexible material. The material may also be impermeable to gas. Examples of suitable materials include silicone or rubber. The flexibility of the membrane 6200 may be affected by a variety of factors, including the material of the membrane and/or the thickness of the membrane, which in some forms may be in the range 0.45 mm-0.55 mm and in some forms may be approximately 0.5 mm. In some forms, as described below, where the membrane moves as pressure in the first volume changes, the flexibility may be a characteristic of the membrane 6200 that affects the rate of vent flow through the vent assembly 6000.

In some forms, the membrane 6200 may be entirely or substantially planar. In alternative forms, the membrane 6200 may comprise a non-planar portion 6220. The non-planar portion 6220 may be contoured inwardly in the direction of vent flow. That is, the side of the non-planar portion 6220 facing towards the first volume may be positively curved (i.e. concave) and the side of the non-planar portion 6220 facing away from the first volume may be negatively curved (i.e. convex). The central region of the membrane 6200 may comprise the non-planar portion 6220. The membrane may have a concave inner surface and a convex outer surface. In the illustrated forms, the concave inner surface is on the side of the non-planar portion 6220 facing towards the first volume, e.g. the internal volume of the respiratory therapy system, and the convex outer surface is on the side of the non-planar portion 6220 facing away from the first volume, i.e. facing the surrounding ambient air. In this way, the non-planar portion 6220 may be substantially dome-shaped, as is the case in the forms of the technology shown in FIGS. 9A-9I. The non-planar portion 6220 may be curved inwardly in the direction of vent flow to form a substantially dome-shaped surface, i.e. the membrane 6200 forms a dome, as is the case in the forms of the technology shown in FIGS. 6A to 6F, 7A to 7F and 8A to 8B. In other forms of the technology, the membrane 6200 may have a different shape, for instance a truncated pyramid with rounded edges. The non-planar portion 6220 of the membrane may be substantially circular in plan view. In other forms, the non-planar portion 6220 may be another shape in plan view, for example oval, square with curved corners or some other polygonal shape, optionally with curved corners.

The membrane 6200 may comprise the non-planar portion 6220 even when there is no pressure difference between the first volume on one side of the membrane 6200 and ambient on the opposite side of the membrane 6200, i.e. the resting position of the membrane 6200 when not in use comprises the non-planar portion 6220. The non-planar portion 6220 may substantially span the vent base aperture 6110. The size of the non-planar portion 6220 may therefore correspond to the size of the vent base aperture 6110.

The non-planar portion 6220 may be the portion of the membrane 6200 that flexes when the pressure of the gas in the first volume acts on the membrane 6200. The non-planar portion 6220 may therefore flex in a direction away from the first volume and towards the cap 6300.

In other forms, the membrane 6200 may be substantially planar in the absence of forces on the membrane, and the force exerted by the pressure of the gas in the first volume may cause the membrane 6200 to flex in a direction away from the first volume and towards the cap 6300.

As explained above, the membrane 6200 may have formed therein a membrane aperture 6210 to allow the vent flow to pass through the membrane 6200. In the form of the technology shown in FIGS. 6A to 6E, the membrane aperture 6210 is located in a central region of the membrane 6200, e.g. on the longitudinal axis of the membrane 6200 in forms in which the membrane 6200 is circular. The surrounding non-central regions of the membrane 6200 are impermeable to gas. The membrane aperture 6210 may be substantially circular in shape. The membrane aperture 6210 may have a central axis aligned with the central axes of one or more of the membrane 6200, the vent cap 6300, and/or the vent base 6100. The membrane aperture 6210 may be located in the non-planar portion 6220 of the membrane 6200. In the form of the technology shown in FIGS. 6A to 6E, the membrane aperture 6210 is located in a region of the dome-shaped membrane 6200 proximate the apex of the dome. The size of the membrane aperture 6220 may affect the rate of vent flow through the vent assembly 6000, and in some forms may have an area of approximately 50-75 mm², or more specifically in some forms may have an area of approximately 57-69 mm².

In the form of the technology shown in FIGS. 7A to 7F, there are a plurality of additional membrane apertures 6210 located closer to the periphery of the membrane 6200. The plurality of additional membrane apertures 6210 may be equally spaced around the periphery. In forms where the membrane 6200 is circular, the additional membrane apertures 6210 may be arranged spaced (e.g. equally spaced) circumferentially, and extend radially and circumferentially. The additional membrane apertures 6210 may all have the same shape, or alternatively may have a number of different shapes. The plurality of additional membrane apertures 6210 may also be substantially rectangular in shape. In other forms, the plurality of additional membrane apertures 6210 may be circular in shape or may be formed by sectors of a circle or annulus sectors. In the form of the technology shown in FIGS. 7A to 7F, the membrane apertures 6210 are formed between rib sections 6250 of the membrane 6200. The rib sections 6250 extend between an inner region of the membrane 6200 surrounding the central membrane aperture 6210 and an outer periphery region of the membrane 6200.

In the embodiment shown in FIGS. 6A to 6E and 9F to 9I the membrane 6200 may additionally comprise a cylindrical portion 6230 that joins to the radially outer region of the non-planar portion 6220. The cylindrical portion 6230 may comprise a flange 6240 on its radially outer periphery that is mounted to the vent base 6100. The cylindrical portion 6230 may serve to position the membrane 6200 as desired in relation to other components of the vent structure.

The shape and structure in combination with the material of the membrane 6200 may be configured such the membrane 6200 is flexible and/or resilient. For example, the membrane 6200 may be formed from a relatively soft material such as silicone or rubber.

In vent assemblies 6000 having one or more flaps 6700, the membrane 6200 and the plurality of flaps 6700 may be integrally formed, as shown in the examples shown in FIGS. 9A-9I. In other forms, the plurality of flaps 6700 may be formed separately and connected to the membrane 6200. In such forms, the plurality of flaps 6700 may be connected to the membrane 6200 in a manner that permits each flap 6700 to move relative to the membrane, for example through bending of the flap 6700. In some forms, the flap 6700 may be rotatably connected to the membrane at a hinged or hinge-like connection. Additionally, or alternatively, the flaps 6700 may be connected to the membrane 6200 via an adhesive or overmoulded. The connection between the flaps 6700 and the membrane 6200 may be sealed so that air cannot pass between these components.

### 4.3.4.4 Plurality of Flaps

The plurality of flaps 6700 may be constructed and arranged to allow the regulation of a vent flow of exhaled gas from the airway of a patient that passes through the vent assembly 6000 to ambient.

In the examples shown in FIGS. 9A-9I, the net flow of air through the vent assembly in use is from the first volume to the surrounding ambient air, as indicated by the straight arrows in FIG. 9C. The straight arrows are oriented parallel to the central longitudinal axis of the membrane 6200, indicating the net flow of air moves in this direction. Generally, in the following description, unless clearly indicated otherwise, when the terms “upstream” and “downstream” are referred to, this is indicating a direction in relation to the net flow of air indicated by the straight arrows in FIG. 9C. Upstream is relatively closer along the flow path to the first volume and further from the surrounding ambient air than downstream, which is relatively further along the flow path from the first volume and closer to the surrounding ambient air.

In the examples shown in FIGS. 9A-9I, the plurality of flaps 6700 extend from the membrane 6200 in a radially inwards direction towards the central aperture 6800. The plurality of flaps 6700 may extend from the perimeter of the membrane aperture. The plurality of flaps 6700 may also extend in a direction towards the flow of air through the vent 3400, i.e. in an upstream direction. That is, the flaps 6700 may be oriented at an acute angle to the general direction of vent flow through the vent assembly 6000, as shown by the series of straight arrows in FIG. 9C. This general direction of vent flow may be parallel to a longitudinal axis of the vent assembly 6000, so each of the flaps 6700 may be oriented at an angle to this axis, at least in their at rest position i.e. when there are no forces acting on the flaps 6700 which may cause them to move position and/or orientation.

The plurality of flaps 6700 may each be shaped like a sheet, such that they are relatively thin in one direction. The plurality of flaps 6700 may have a curved shape, i.e. a non-planar shape. The plane of the sheet of the plurality of flaps 6700 may be curved in one or more direction, for instance it may curve towards the first volume, i.e. the inner surface of the flaps 6700 (located on an upstream side of the flaps 6700 and facing the first volume) may be concave and the outer surface of the flaps (opposite the inner surface, located on a downstream side of the flaps 6700 and facing away from the first volume) may be convex. The curved shape of the plurality of flaps is shown in cross-section in FIGS. 9C and 9I and described further below.

The thickness of the flaps 6700 may be similar to that of the membrane 6200 and, in some forms the inner surface of the flaps 6700 may be contiguous with the inner surface of the membrane 6200 and the outer surface of the flaps 6700 may be contiguous with the outer surface of the membrane 6200. In the illustrated forms, the inner and outer surfaces of the plurality of flaps 6700 have a shape of a sector of an annulus in plan view (e.g. when projected onto a plane), while in other forms they may have another shape, for instance square, trapezoidal, triangular or rectangular when projected onto a plane.

The plurality of flaps 6700 may have a substantially arc-shaped cross-section as shown in FIG. 9I. The curvature of the inner surface of the flaps 6700 may be greater than a curvature of the outer surface of the flaps 6700. In cross-section, as shown in FIG. 9I, the outer surface may have a greater curvature than the inner surface, i.e. the curvature of the outer surface may be greater than the curvature of the inner surface. This may mean that the arc-length of the outer surface of the flaps 6700 in cross-section (as shown in FIGS. 9C and 9I) may be longer than the arc-length of the inner surface of the flaps 6700 in the same cross-section.

Each flap 6700 may have a thickness that varies along its length. For example, the flaps 6700 may taper towards their radially inner end, i.e. they may have a greater thickness at a radially outer region compared to a radially inner region. Due to the orientation of the flaps, this means that the downstream portions of the flaps 6700 may have a greater thickness than the upstream portions. The taper of the flaps 6700 may assist flaps 6700, in particular the radially inner end of the flaps 6700, to be more responsive to pressure changes in the first volume compared to the thicker, radially outer portions of the flaps, i.e. the radially inner end of the flaps 6700 may move more easily in response to pressure changes in the first volume than the radially outer portions of the flaps. The Durometer hardness of the material forming the flaps 6700 may also affect how responsive the flaps 6700, or portions of the flaps 6700, are to pressure changes. For instance a harder flap 6700, or portion of the flap 6700, may be stiffer and therefore less responsive to pressure changes in the first volume compared to a less hard flap 6700, or portion of the flap 6700.

In different forms of the technology, the shape, including the thickness and arc shape, of the plurality of flaps 6700 may be selected based on the amount the flaps 6700 are desired to move as pressure changes. This is discussed further below.

In certain forms, each of the plurality of flaps 6700 may have the same shape and size as the other flaps, as in the illustrated examples, while in other forms of the technology the flaps may have different shapes and/or sizes to alter the function of the flaps 6700 and therefore the flow of air through the vent assembly 6000.

In certain forms, the plurality of flaps 6700 are arranged to form the central aperture 6800 between their radially inward free ends (which may take the form of a tip), and may be located around an outer circumference of the central aperture 6800. The central aperture 6800 may be substantially circular in cross-section in some forms, while in other forms it may have an alternate shape, for instance square, rectangular, or oval. The shape of the central aperture 6800 may be determined by the shape of the plurality of flaps 6700, particularly their free ends. In the forms of the technology illustrated, the portion of the plurality of flaps 6700 adjacent the central aperture may have an arc-shape, curved circumferentially around the longitudinal central axis of the membrane 6200. The arc-shape can be seen in FIGS. 9A and 9B and when the plurality of flaps 6700 are viewed from the top or bottom, i.e. in or opposing the direction of the flow of air. The arc-shapes are configured such that they together form a substantially circular perimeter that forms the central aperture 6800.

Each of the plurality of flaps 6700 may have a fixed end 6701 that is connected to the membrane 6200, for example to an inner surface of a radially inner region of the membrane 6200, or to another portion of the respiratory therapy system for instance the inner surface in a wall of the plenum chamber 3200 or seal-forming structure 3100. That is, in some forms, the vent structure 3400 may be absent a membrane and the flaps 6700 may be mounted directly in an aperture of another component of the patient interface 3000. Each of the plurality of flaps 6700 may also have a free end 6702 that is opposite the fixed end 6701 and is not connected to anything. The fixed end 6701 may be a downstream portion of the flaps 6700 and the free end 6702 may be an upstream portion of the flaps 6700 relative to the straight arrows of FIG. 9C. As the plurality of flaps 6700 move between the first and second configurations they may rotate about the fixed ends 6701. The plurality of flaps 6700 may rotate about the fixed ends 6701 in a direction downstream or away from the first volume as they are urged by the pressure of air in the first volume from the second configuration to the first configuration. The plurality of flaps 6700 may rotate about the fixed ends 6701 in a direction upstream or towards the first volume as they move from the first configuration to the second configuration.

In some forms, alternatively or in addition to the rotation about the fixed ends 6701, the shape of the flaps 6700 may change between the first configuration and the second configuration, for example through elastic deformation of the flaps 6700. In some forms the curvature of the plurality of flaps 6700 may decrease as they move from the second configuration to the first configuration, i.e. the flaps 6700 may become less arched or may flatten due to the action of the pressurised air in the first volume.

In use when there is no pressurised flow of air provided to the first volume, the plurality of flaps 6700 may be in a rest configuration. In use the plurality of flaps 6700 may be able to move between a plurality of configurations, or a continuum of configurations, as the pressure in the first volume changes, whereby each of the different configurations provide a different area of the central aperture 3800 and therefore vary the amount of flow of air exiting the vent 3400. The first and second configurations described earlier are configurations of the plurality of configurations.

In some forms the shape and the structure in combination with the material of the plurality of flaps 6700 may be configured such that the flaps 6700 are resilient such that they return towards the rest position when the pressure in the first volume decreases. In other forms, a spring mechanism may act on the flaps such that they return towards the rest position when the pressure in the first volume decreases.

In the example shown in FIGS. 9A-9I, the plurality of flaps 6700 are separate from each other, i.e. they are not connected along their side edges (which may be aligned radially, as explained below). The plurality of flaps 6700 are separated by a plurality of slits 6710 formed between the plurality of flaps 6700. The plurality of slits 6710 may extend from the central aperture 6800 to the edge of the membrane 6200 adjacent the plurality of flaps 6700. The flaps 6700 may be oriented radially with respect to the circular geometry membrane 6200. In some forms of the technology where the membrane 6200 and the plurality of flaps 6700 are integrally formed, the plurality of slits 6710 may be formed in the membrane 6200 such that the plurality of flaps 6700 are formed therebetween. In use, when the plurality of flaps 6700 move into the first configuration, the free ends of the plurality of flaps 6700 may be pushed together and the size of the slits 6710 may decrease, to decrease the size of the central aperture 6800.

The shape and structure of the flaps 6700, in combination with the material of the plurality of flaps 6700 may be configured such the plurality of flaps 6700 are flexible and/or resilient. In certain forms, the plurality of flaps 6700 may be formed from a silicone or rubber, for example.

In certain forms of the technology, the vent assembly 6000 may also comprise a vent cap 6300 mounted to the vent base. The vent cap 6300 may be located downstream of the membrane 6200 and the plurality of flaps 6700 relative to the vent flow. The vent cap 6300 may be positioned in the path of the vent flow after it has passed through the central aperture 6800.

In other forms, for example the form shown in FIGS. 9A to 9C, the vent assembly 6000 has no vent cap.

### 4.3.4.5 Vent Cap

In examples of the present technology in which the vent assembly 6000 comprises a vent cap 6300, the vent cap 6300 may be mounted to the vent base 6100 and located downstream of the membrane 6200 relative to the vent flow. The vent cap 6300 may be positioned in the path of the vent flow through the membrane aperture 6210. The vent cap 6300 may therefore be configured to direct vent flow through at least a part of the vent assembly 6000. In some forms, a first surface, inner surface 6310 of the vent cap 6300 faces towards the membrane 6200. The first surface 6310 may be in contact with vent flow that has passed through the membrane aperture 6210 and flows through the space between the vent cap 6300 and the membrane 6200. In some forms, as shown in FIGS. 8A and 8B, a second, outer surface 6320 of the vent cap opposite the first surface 6310 faces away from the membrane 6200. This surface may also face away from the patient when the patient interface 3000 is worn. The second surface 6320 may be in contact with the surrounding ambient air.

As shown in the embodiments in FIGS. 6A to 6E, 7A to 7F, and 9I, the first surface 6310 of the vent cap 6300 facing the membrane 6200 may have a shape substantially corresponding to that of the membrane 6200, for instance the first surface may have substantially the same or similar contours as the membrane 6200. For example, the first surface 6310 may have a shape that substantially corresponds to the non-planar portion 6220 of the membrane 6200 . . . . For instance, in examples where the outer surface of the non-planar portion 6220 is convex, the inner surface of the vent cap 6300 may be concave. For example, where the non-planar portion 6220 is dome shaped, the first surface 6310 is inverted dome shaped.

In some forms, the second, outer surface 6320 has a different shape to the first surface 6310. In some forms, for instance that shown in FIGS. 8A and 8B, the second surface 6320 is substantially planar with a flat surface oriented perpendicular to the direction of vent flow entering the vent base aperture 6110. In other forms, the second surface 6320 may have a substantially similar shape as the first surface 6310, only on the other side of the vent cap 6300 and therefore be convex instead of concave. For instance, in the form of the technology, shown in FIGS. 6A to 6E, the second surface 6320 is also dome shaped such that the entire vent cap 6300 is dome shaped with the second surface 6320 being the convex surface that is complementary to the convex-shaped surface of the first surface 6310.

In certain forms of the technology, the first, inner surface 6310 may be a substantially smooth surface, such that it reduces the amount of turbulence created in the vent flow as it passes along adjacent to the first surface 6310 towards the vent outlet(s) 6400 compared to forms in which the first surface 6310 has protrusions or ridges. In some forms, the vent flow is directed by the vent cap towards an outer periphery of the vent cap 6300 where the vent outlet(s) 6400 are located. In some forms, as shown in FIGS. 8A and 8B, the first surface 6310 around the periphery of the vent cap 6300 is substantially planar and has a flat surface perpendicular to the direction of vent flow entering the vent base aperture 6110. The region of the first surface 6310 that is substantially flat may be the region that faces an upper surface of the vent base 6100.

In some forms, the vent flow exits the vent assembly 6000 in a direction perpendicular to the direction of vent flow entering the vent base aperture 6110. This direction may be parallel to the surface of the surrounding patient interface 3000, for instance the plenum chamber 3200. In other forms, like that shown in FIGS. 6A to 6E and 9F to 9I the first surface 6310 is contoured, in this case dome-shaped, even at the peripheral regions of the vent cap 6300. The vent flow therefore exits the vent outlet(s) in a direction towards the vent base 6100. In the form shown in FIGS. 6A to 6E, an upper region 6120 of the vent base 6100 adjacent the periphery of the membrane 6200 may also be contoured in a shape that follows the membrane 6200, such that the membrane 6200 and the upper region 6120 have substantially the same or a similar dome-shape as the vent cap 6300. The region where the periphery of the vent membrane 6200 and the upper region 6120 meet may be aligned such that the surface of the flow path formed by the outer surface of the vent membrane 6200 and the outer surface of the vent base 6100 may be substantially continuous, i.e. the edges do not meet at an angle unlike in the form shown in FIGS. 8A and 8B. This may assist in reducing turbulence of the vent flow through the vent assembly 6000. Reducing turbulence may be beneficial in reducing the amount of noise generated by the vent.

The vent cap 6300 may substantially cover the membrane 6200 forming a vent gap 6600 therebetween, discussed further below. In alternative embodiments, the vent cap 6300 may be substantially planar and have a recess in the surface of the vent cap 6300 facing the membrane 6200. The recess may have a shape that substantially corresponds to the non-planar portion 6220 of the membrane 6200.

The vent cap 6300 is formed from a substantially hard material and constructed in a shape that results in the vent cap being substantially inflexible when subject to the forces encountered during typical use, and in some forms may be formed from a polycarbonate. The vent cap 6300 therefore may have a constant shape during pressure changes in the first volume. The vent base 6100 may also be formed from the same material as the vent cap 6100 or another material and in a shape that renders the vent base 6100 similarly inflexible during typical use.

The vent cap 6300 may be mounted to the vent base 6100 at a region around the periphery of the vent cap 6300. In some forms of the technology the vent cap 6300 is mounted at a single region on the periphery of the vent cap 6300. In other forms the vent cap 6300 may be mounted at a plurality of regions on the periphery of the vent cap 6300. The vent cap 6300 may be mounted to the base by several struts between the periphery of the vent cap 6300 and the upper region 6120 of the vent base 6100 to form gaps between the outer periphery of the vent cap 6300 and the vent base 6100. The gaps formed between these struts may form vent outlets 6400. In the form of the technology as shown in FIGS. 7A to 7F, the vent cap 6300 may be mounted to the vent base (not shown) around the entirety of the periphery of the vent cap 6300. The vent cap 6300 may be mounted to the vent base 6100 via one of a clip mechanism, a snap-fit mechanism, or by being integrally formed, by way of example only.

FIGS. 9F-9I show a vent assembly 6000 according to one form of the technology having flaps 6700 in which a vent cap 6300 is provided. In this form, the vent assembly 6000 has a generally cylindrical geometry with the vent base, membrane 6200, and vent cap 6300 all having a circular cross-sectional shape with aligned longitudinal axes. In other forms of the technology, the vent assembly 6000 may have a different geometrical layout, for instance having cuboidal or truncated pyramidal geometry. The vent base, membrane 6200, and vent cap 6300 may have a corresponding cross-sectional shape to that of the vent assembly 6000 or one or more components may have a different shape.

### 4.3.4.5.1 Movement of the Membrane

In some forms of the vent assembly 3400, in use, the membrane 6200 may be sufficiently flexible that the pressure of the gas in the first volume acts on the membrane 6200 and causes the membrane 6200 to flex, e.g. bulge outwardly, particularly the non-planar portion of the membrane, particularly at higher pressures. The varying flex in the membrane 6200 varies a position of the membrane 6200 or a portion or portions thereof relative to the vent cap 6300, which changes the height of the vent gap 6600. As described above, this mechanism (in addition to or combination with the movement of any flaps 6700 that may be present) may be used to control the vent flow through one or more vent outlets to ambient, e.g. the rate of vent flow. Generally, when the pressure of the gas in the first volume increases the membrane 6200 flexes into a position closer to the vent cap 6300 and such that there is a reduced vent flow through the vent outlet(s) than would be the case at the same pressure if the membrane did not move closer to the vent cap.

In these forms of the technology, as the vent membrane 6200 flexes the size (e.g. cross sectional area) of the vent gap 6600 changes which varies the amount of vent flow through the vent gap 6600 and therefore the rate of vent flow exiting the vent outlet. The vent cap 6300 and the membrane 6200 may be positioned far enough apart such that the membrane 6200 never fully closes or blocks the vent gap 6600, even at relatively high pressures typically encountered during use in the first volume. Similarly, the central aperture 6800 may never fully close or be blocked off by the plurality of flaps 6700.

The way in which the configuration of the membrane 6200 varies with changes in pressure, and consequently the way in which the vent flow rate changes with pressure, may be determined based on certain characteristics of the membrane 6200. Examples of such characteristics and how they may be varied will be discussed further below. In certain forms, the vent assembly 6000 may be configured so that, in use, the vent flow rate of exhaled air from the first volume through the vent assembly 6000 to ambient is substantially constant for a range of pressures inside the first volume.

In some forms, the membrane 6200 may be substantially planar in the absence of forces on the membrane, and the force exerted by the pressure of the gas in the first volume may cause the membrane 6200 to flex such that the size and shape of the membrane aperture and/or the vent gap 6600, as the case may be, changes.

### 4.3.4.6 Vent Outlet(s)

The vent structure 3400 may comprise or define one or more vent outlets where the vent flow from the respiratory pressure therapy system leaves the vent structure 3400 to ambient.

In the form of the technology shown in FIGS. 6A to 6E the vent cap 6300 is mounted to the vent base 6100 to form the one or more vent outlets 6400 between the vent cap 6300 and the vent base 6100. In the form of the technology shown in FIGS. 6A to 6E, the vent flow passes through the membrane aperture 6210 and is directed by the vent cap 6300 into a flow path formed as the vent gap 6600. The vent gap 6600 is formed between the vent membrane 6200 and the vent cap 6300. The vent flow passes through the vent gap 6600 and then through the vent outlet 6400. As the vent membrane 6200 flexes the size of the vent gap 6600 changes which varies the amount of vent flow through the vent gap 6600 and therefore the rate of vent flow exiting the vent outlet 6400. The vent cap 6300 and the membrane 6200 are positioned far enough apart such that the membrane 62000 never fully closes or blocks the vent gap 6600 even at relatively high pressures typically encountered during use in the first volume.

In some forms of the technology, a single vent outlet 6400 is formed around at least a portion of the periphery of the vent cap 6300. In other forms of the technology, one or more struts may be formed between the vent cap 6300 and the vent base 6100 around the periphery of the vent cap 6300 to form a plurality of vent outlets 6400. The struts may be equally spaced around the periphery of the vent cap 6300. The struts may help to separate the flow paths of the vent flow and the struts may have a streamlined cross-sectional shape, which may assist in reducing turbulence and noise of vent flow exiting the vent assembly 6000. The vent flow of gas may additionally or alternatively be discharged form vent cap holes 6301 or from a central hole 6302 shown in FIGS. 6A, 6B and 6D. In other examples the central hole 6302 may not be provided, may be plugged by a different component or may provide a flow path for air supply into a plenum chamber 3200 of the patient interface 3000.

In the example shown in FIGS. 8A and 8B the vent assembly 6000 comprises a pair of vent outlets 6400 formed by gaps between the vent base 6100 and the vent cap 6300. Each one of the pair of vent outlets 6400 in this example extends around substantially half of the circumference of the vent assembly 6000. The vent outlets 6400 are separated from each other around the circumference only by a pair of struts connecting the vent base 6100 and vent cap 6300.

In the form of the technology shown in FIGS. 7A to 7F, the one or more vent outlets 6400 are formed as a plurality of cap apertures 6330 in the vent cap 6300. The cap apertures 6330 may be distributed across the vent cap 6300, for example substantially evenly distributed. The membrane 6200 and the vent cap 6300 are configured so that varying the flex of the membrane 6200 varies the number of the plurality of cap apertures 6330 that are blocked by the membrane 6200 to restrict the vent flow of gas to ambient through the plurality of cap apertures 6330. In forms of the technology, the rib sections 6250 that are formed around the additional membrane apertures 6210 are aligned with one or more of the cap apertures 6330. The rib sections 6250 may move closer to the cap apertures 6330 as the pressure of the gas in the first volume increases, until the rib sections eventually cover the cap apertures 6330, completely blocking vent flow through cap apertures 6330. In forms such as that shown in FIGS. 7A to 7F, the vent cap 6300 and the membrane 6200 are positioned such that the vent gap 6600 between them can be closed as the pressure increases. As the degree of flex of the membrane 6200 increases, the apertures of the plurality of cap apertures 6330 located closer to a periphery of the vent cap 6300 are blocked by the membrane 6200 prior to cap apertures 6330 of the plurality of cap apertures 6330 located further from the periphery of the vent cap 6300. The number of cap apertures 6330 blocked by the membrane 6200 may regulate the vent flow through the vent assembly 6000. In other forms of the technology, as the amount of flex of the membrane 6200 increases, the cap apertures 6330 of the plurality of cap apertures 6330 located further to a periphery of the vent cap 6300 are blocked by the membrane 6200 prior to apertures of the plurality of apertures located closer to the periphery of the vent cap 6300. The plurality of apertures may be equally spaced and located concentrically around the vent cap 6300.

In some forms of the technology, as shown in FIGS. 9A-9E, the vent outlet is the central aperture 6800 formed by the plurality of flaps 6700.

### 4.3.4.7 Operation of the Vent Assembly

In the form of the technology shown in FIGS. 6A to 6E, 8A and 8B, the vent assembly 6000 operates as follows. The air exhaled by the patient is initially received by the vent assembly 6000 through the vent base aperture 6110. The vent flow passes through the membrane aperture 6210 which is in a central region of the membrane 6200. The vent flow then contacts the vent cap 6300 which is positioned in the path of the vent flow after it passes through the membrane aperture 6210. The first surface 6310 of the vent cap 6300 that faces the membrane 6200 then causes the vent flow to turn radially outwardly through a vent gap 6600 formed between the membrane 6200 and the vent cap 6300. The vent cap 6300 directs the vent flow towards the vent outlet 6400 which is located between an upper region 6120 of the vent base and a periphery of the vent cap 6300.

As the pressure of the gas in the first volume increases, the membrane 6200 flexes in the direction of the vent flow to reduce the size of the vent gap 6600 to reduce the vent flow exiting the vent outlet 6400. Similarly, as the pressure of gas in the first volume decreases, the membrane 6200 relaxes, increasing the size of the vent gap 6600. This allows the vent assembly 6000 to be configured in a manner that means the vent flow at a given pressure can be controlled. For example, in some forms, the vent assembly 6000 may be configured so that a relatively constant flow exiting the vent assembly 6000 may be achieved for a variety of pressures in the first volume. The pressure-flow relationship of the form of the technology in FIGS. 8A and 8B is shown in FIG. 8C, which shows that the flow exiting the vent for three different samples remains between 8 to 13 L/min for pressures from 4 cmH₂O to 25 cmH₂O. The vent flow rates at high pressures are lower than would be the case if the membrane 6200 did not reduce the size of the vent flow path as the pressure in the first volume increased.

In the form of the technology shown in FIGS. 7A to 7F, the vent assembly 6000 operates as follows. The air exhaled by the patient is initially received by the vent assembly 6000 through the vent base aperture (not shown). The vent flow then passes through a plurality of membrane apertures 6210 into a vent gap 6600, shown in FIG. 7F, formed between the membrane 6200 and the vent cap 6300. The vent flow then passes through a plurality of vent outlets 6400 formed as cap apertures 6330 in the vent cap 6300. The vent flow therefore exits in a direction away from the surrounding patient interface 3000. As the pressure of the gas in the first volume increases, the membrane 6200 flexes in the direction of the vent flow to reduce the size of the vent gap 6600 and sequentially block or cover a greater proportion of the cap apertures 6330. This reduces the vent flow exiting the vent outlets 6400. Similarly, as the pressure of gas in the first volume decreases, the membrane 6200 relaxes, uncovering some of the cap apertures 6350. Therefore the vent assembly 6000 can be configured to achieve the desired pressure-flow relationship for vent flow passing through the vent assembly 6000 in relation to the pressure of gas in the first volume. For example, in some forms, the vent assembly 6000 may be configured to maintain a relatively constant flow exiting the vent assembly 6000 for a variety of pressures in the first volume.

In the form of the technology shown in FIGS. 9A-9E, the vent assembly 6000 operates as follows. The air exhaled by the patient is initially received by the vent assembly 6000 through the vent base aperture. The vent flow passes through the central aperture 6800 which is formed by the plurality of flaps 6700. As the pressure of the gas in the first volume increases, the plurality of flaps 6700 move or flex in the direction of the vent flow to reduce the size of the central aperture 6800. This reduces the vent flow exiting the vent compared to if the aperture was larger at the same pressure. Similarly, as the pressure of the gas in the first volume decreases, the plurality of flaps 6700 may be configured to move or flex in a direction towards the flow of air, increasing the size of the central aperture 6800. As discussed above, the plurality of flaps 6700 may be biased towards a rest position, via resilience in the flaps 6700 or a spring mechanism, such that when there is less pressure acting on the flaps 6700 they move towards the rest position in which the size of the central aperture 6800 is larger than when there greater pressure acting on the flaps 6700 causing them to move or flex. Flow of air from the central aperture 6800 may directly exit to the surrounding ambient air, as shown in FIG. 9D. In some forms of the technology, as shown in FIG. 9E, rigidiser arms 3310, discussed below, or other portion of the patient interface 3000, may comprise an aperture through which the flow of air from the central aperture 6800 passes before exiting to the surrounding ambient air. This allows the vent assembly 6000 to be configured in a manner that means the vent flow at a given pressure can be controlled. For example, in some forms, the vent assembly 6000 may be configured so that a relatively constant flow exiting the vent assembly 6000 may be achieved for a variety of pressures in the first volume.

In the forms of the technology shown in FIGS. 9F-9I, the operation of the vent assembly 6000 is similar to as just described with reference to the form of FIGS. 6A to 6E but, in addition, the vent flow, having passed through the central aperture 6800, contacts the vent cap 6300 which is positioned in the path of the vent flow after it passes through the central aperture 6800. The first surface 6310 of the vent cap 6300 that faces the membrane 6200 then causes the vent flow to turn radially outwardly through a vent gap 6600 formed between the membrane 6200 and the vent cap 6300. The vent cap 6300 directs the vent flow towards the vent outlet which may be located between an upper region of the vent base and a periphery of the vent cap 6300 or alternatively in a connector portion 6500, which is described below. With reference to FIG. 9I, a vent flow of gas may additionally or alternatively flow through the vent cap holes 6301.

### 4.3.4.8 Connection to Headgear

As mentioned above, FIGS. 6A-6E and 9A-9I show a vent assembly 6000 that may in use be located in the plenum chamber 3200, as shown in FIGS. 6E, 9D and 9E. The vent assembly 6000 in these embodiments comprises a further connector portion 6500 which is configured to connect the vent assembly 6000, and therefore the plenum chamber 3200, to a portion of the positioning and stabilising structure 3300. The connector portion 6500 may be mounted to either the vent cap 6300 or vent base 6100. For example, the connector portion 6500 may be mounted to the side of the vent cap 6300 facing away from the membrane 6200. In the forms of the technology shown in FIGS. 6E and 9F-9I, the connector portion 6500 is integrally formed as part of the side of the vent cap 6300 facing away from the membrane 6200.

In some forms, the flange 6240 may connect directly to the positioning and stabilising structure 3300, and in some forms directly to the rigidiser arms 3310. A portion of the flange 6240 may connect to an end region of the rigidiser arms 3310.

In the form of the technology shown in FIG. 6F, the connection portion, or in other forms the vent base, makes a friction fit connection to a pair of rigidiser arms 3310 that form part of the positioning and stabilising structure 3300. In other forms of the technology, the connection portion may connect to the positioning and stabilising structure 3300 via one of a snap-fit connection, or glue or adhesive, or by having a loop through which a portion of the positioning and stabilising structure 3300 can pass through and then reattach to itself.

### 4.3.4.9 Tuning the Vent

It has already been explained that the vent flow rate of a vent assembly 6000 according to certain forms of the technology may depend on the pressure of the gas within the first volume. The manner in which the vent flow rate depends on the pressure may be known as the pressure-flow relationship of the vent assembly 6000. The pressure-flow relationship for any vent assembly 6000 may be determined by certain aspects of the configuration of the membrane 6200 of that vent assembly 6000. When designing or manufacturing a vent assembly 6000, any one or more of those aspects of the configuration may be selected in order to provide the desired pressure-flow relationship. The selection of these characteristics, and configuring the membrane 6200 accordingly, may be referred to as tuning the vent assembly 6000. Tuning a vent assembly 6000 enables the vent flow rate for any given air pressure inside the first volume to be selected as desired.

In some forms, tuning the vent assembly 6000 may involve configuring the vent assembly 6000 to provide the desired pressure-flow relationship during inhalation and, separately, the desired pressure-flow relationship during exhalation.

The desired pressure-flow relationship may be determined based on various factors including, but not limited to: the nature of the patient interface 3000; the nature of the RPT device 4000; a patient's treatment preferences; a clinician's treatment preferences; and/or the nature of the respiratory treatment.

One factor that affects the vent flow rate at a given pressure, and therefore the overall pressure-flow relationship of the vent assembly 6000, is the degree that the vent outlet(s) 6400 are occluded at the given pressure. Two non-limiting examples of design characteristics that can affect the level of occlusion of the vent outlet(s) 6400 at a given pressure include: the ratio between the effective vent opening area when the membrane 6200 is flexed compared to when it is in a neutral position; and the resistance of the membrane 6200 to flexing. Exemplary ways in which these characteristics may be varied in different forms of the technology will now be described. These design variants, as well as others not described herein, may be used separately or in any combination together.

It will be understood that the term “effective vent opening area” as used herein may refer to the effective area through which the vent flow may pass in order to escape the vent assembly 6000 to ambient. This area may be affected by the number, size, shape and positioning of the vent outlets 6400 and also the degree to which the vent outlet(s) 6400 or the vent gap 6600 are occluded. For example, in the forms of the technology shown in FIGS. 6A to 6E and 8A to 8B, a high level of occlusion (e.g. by the vent membrane 6200 flexing to a greater extent) reduces the size of the vent gap 6600 through which the vent flow must pass through in order to reach the vent outlet 6400, thus reducing the effective area of the vent.

In forms of the technology where the membrane moves, a high level of occlusion (e.g. by the vent membrane 6200 flexing to a greater extent) reduces the number of vent outlet(s) or cap apertures 6330 that are open to the ambient, thus reducing the effective area of the vent. In some forms, the effective area of the vent may also be affected by the membrane apertures 6210. The number, shape, size and/or positioning of these apertures may be selected to achieve the desired pressure-flow characteristics.

The effective area of the vent may also be affected by the size of the vent gap 6600, i.e. the distance between the vent cap 6300 and the membrane 6200. Increasing this distance will generally increase the size of the effective area of the vent. The size of the vent gap 6600 may be selected to achieve the desired pressure-flow characteristics.

Characteristics of the membrane 6200 and/or the plurality of flaps 6700 may also be selected to achieve the desired pressure-flow characteristics. These characteristics of the membrane 6200 include but are not limited to: the material the membrane 6200 and/or the plurality of flaps 6700 is formed from, in particular the stiffness and/or hardness of the material; the thickness of the membrane 6200 and/or and the plurality of flaps 6700, in particular the thickness of the non-planar region 6220; and the shape of the membrane 6200 and/or and the plurality of flaps 6700, for instance the maximum height of the dome region in forms of the technology where the non-planar region 6220 is dome shaped and the curvature of the plurality of flaps 6700.

As has been explained, in certain forms of the technology, characteristics of the vent assembly 6300 may be selected in order to achieve a pressure-flow relationship in which the vent flow rate of the flow of exhaled air from the first volume through the vent assembly 6300 to ambient is substantially constant for a range of pressures inside the first volume or at least increases with increased pressure to a lesser extent than it would in a vent assembly with no moving parts. In some forms, the vent structure 3400 may be configured so that the pressure-flow relationship may remain between 8 to 13 L/min for pressures from 4 cmH₂O to 25 cmH₂O.

### 4.3.4.10 Plunger Vents

Vents in some forms of the present technology comprise a plunger 6140 moveably positioned with respect to a vent body 6130 to define a regulated vent flow passage 6132. The plunger 6140 may also be identified as a moveable portion. The plunger 6140 may be a moveable portion of the vent assembly 6000 that moves relative to another portion of the vent assembly 6000, for example the vent body 6130 or a portion thereof, for example to change a size or cross section of a passage through which gas can flow. In some examples, the plunger 6140 may move in translation, for example along a central axis of the vent assembly 6000 or a portion thereof. In some examples the plunger 6140 may move towards or into a vent body aperture 6138 in use. Some or all of the plunger 6140, for example a portion of the plunger 6140 that defines the regulated vent flow passage 6132, may be substantially rigid. The plunger 6140 may be connected to a flexible portion which is connected to a portion of the vent assembly 6000, the flexible portion allowing the plunger 6140 to move within the vent assembly 6000. As will be described, in some examples the flexible portion may be a membrane 6200.

FIGS. 10A-10D show a vent assembly 6000 according to an example of the present technology. The vent assembly 6000 is for a respiratory therapy system for providing respiratory pressure therapy to a patient. The vent assembly 6000 is configured in use to convey a vent flow of gases from a first volume interior to the respiratory therapy system to ambient. FIGS. 11, 12A-12B, 13A-13D, 14A-14E, 15A-15B. 16A-16C, 17A-17D, 18A-18C, 19A-19C and 20A-20C show similar vent assemblies 6000. Any of the vent assemblies 6000 described herein may be configured to convey a vent flow of gases from the first volume to atmosphere continuously throughout a respiratory cycle of the patient, for example to provide for continuous gas washout of a plenum chamber 3200 of a patient interface 3000. This differs from some other types of vents which open and close during the respiratory cycle, such as opening during exhalation but closing during inhalation. That said, in some other examples of the present technology the vent assembly 6000 may open and close during a single respiratory cycle.

As illustrated in FIGS. 10A-10D, the vent assembly 6000 comprises a vent body 6130 fluidly connected in use to the first volume of the respiratory pressure therapy system and comprising a vent body aperture 6138 through which the vent flow of gases flows in use towards ambient. The vent assembly 6000 further comprises a plunger 6140 positioned with respect to the vent body 6130 to define a regulated vent flow passage 6132 for the vent flow of gases between the plunger 6140 and the vent body 6130. The plunger 6140 is moveable with respect to the vent body 6130 and in this example the plunger 6140 is biased towards a rest position. In the rest position, the regulated vent flow passage 6132 may open. FIG. 10D shows a cross section view through a vent assembly 6000 in which the plunger 6140 is in a rest position and the regulated vent flow passage 6132 is open.

In use, changes in pressure of gas in the first volume cause changes in the position of the plunger 6140 relative to the vent body 6130. The pressure of gas in the first volume may act directly on the plunger 6140 or on another component attached to the plunger 6140, such as a membrane 6200, which will be described below. The plunger 6140 may begin to move when the pressure in the first volume becomes greater than the pressure acting on an opposing side of the plunger 6140, which may be ambient/atmospheric pressure or otherwise a lower pressure than the pressure of the first volume. The plunger 6140 may stop moving towards the vent body 6130 when biasing forces acting on the plunger 6140 (e.g. caused by elastic deformation of a membrane 6200 or other biasing means, to be described below), become sufficiently large to balance the force on the plunger 6140 caused by the pressure difference on opposing sides of the plunger 6140.

The vent assembly may be structured and arranged such that changes in pressure of gas in the first volume cause changes in a position of the plunger 6140 relative to the vent body 6130 to regulate the vent flow of gases through the regulated vent flow passage 6132 throughout a therapeutic pressure range. With increased pressure in the first volume, the plunger 6140 moves towards the vent body to reduce a cross sectional area within the regulated vent flow passage 6132, preventing a corresponding increase in volumetric vent flow rate or causing a lesser increase in volumetric vent flow rate than would otherwise be produced by a vent flow passage of fixed size. Regulation of vent flow rate and its advantages is discussed in detail above with reference to the vent assemblies shown in FIGS. 6A-9I. The vent assemblies 6000 shown in FIGS. 10A-27E and 29A-29D are further examples of vents which at least partially regulate vent flow and the disclosure of these vent assemblies is to be read together with the discussion on regulation of vent flow rate made above with reference to FIGS. 6A-9I.

The therapeutic pressure range throughout which a vent assembly 6000 regulates the vent flow of gases may be, in some examples, between 2 and 30 cmH₂O or, in some examples, between 4 and 20 cmH₂O.

As shown in FIGS. 10C and 10D, the plunger 6140 may comprise a frustoconical portion 6144 and the vent body 6130 may comprise a frustoconical portion 6134. The frustoconical portions 6134, 6144 may together define the regulated vent flow passage 6132. In use when the position of the plunger 6140 relative to the vent body 6130 changes, the frustoconical portion 6144 of the plunger 6140 moves with respect to the frustoconical portion 6134 of the vent body 6130 to change a cross-sectional area of the regulated vent flow passage 6132. The walls of the plunger 6140 move closer to the opposing walls of the vent body 6130 to reduce the size of the regulated vent flow passage 6132. The reduced cross-sectional area means that despite an increase in pressure in the first volume relative to ambient, the amount of gas that flows through the regulated vent flow passage does not increase, or does not increase as much as it would if there was no change in cross-sectional area.

In the example shown in FIGS. 10A-10D the vent assembly 6000 comprises a connecting portion moveably connecting the plunger to the vent body 6130. In this particular example, the connecting portion comprises at least one flexible membrane 6200 supported by the vent body 6130. The plunger 6140 is attached to the membrane 6200 and changes in pressure of gas in the first volume cause deformation of the membrane 6200 causing movement of the plunger 6140. In this example the gas pressure in the first volume may act on the membrane 6200, and a portion of the plunger 6140. The membrane 6200 may deform by bending or stretching, for example. In this example the membrane 6200 is disc shaped with a plurality of membrane apertures 6210 to allow gas to flow into the regulated vent flow passage 6132. The membrane 6200 is supported at its periphery by the vent body 6130. In this example the membrane 6200 fits over a peripheral portion of the vent body 6130. The membrane 6200 may deform in a region between its periphery, which may be fixed to the vent body 6130 and is centre, which may be attached to the plunger 6140.

In the example shown in FIGS. 10A-10D, upon deformation of the membrane 6200 the plunger 6140 moves in a direction aligned with a central axis of the vent body 6130. The vent body 6130, as illustrated, is generally cylindrical and the central axis may be the central axis of the generally cylindrical shape. The plunger 6140 and membrane 6200 may each also comprise a respective central axis, each of which in this example is aligned with the central axis of the vent body 6130.

The vent assembly 6000 shown in FIGS. 10A-10D, also comprises a diffuser 6150. The diffuser 6150 may be positioned such that the vent flow of gases is incident on the diffuser 6150 downstream of the vent body aperture 6138. The diffuser 6150 may be configured to diffuse some or all of the vent flow of gases flowing through the vent assembly 6000. The diffuser 6150 may muffle noise produced by the vent flow of gases or prevent noise being created by slowing and diffusing the flow of gases. The diffuser 6150 may be formed from felt, textile, foam or the like. In some examples disclosed herein, the vent assembly 6000 may be configured so as to allow some of the vent flow of gases to bypass the diffuser 6150, which advantageously may allow the vent assembly 6000 to continue to function even in the event the diffuser 6150 becomes blocked, e.g. clogged with moisture, other fluids and/or dirt etc. As shown in FIG. 10D for example the diffuser 6150 does not fill the entire width of the interior of the vent assembly 6000, so that gas can flow around the outside of the diffuser 6150 if unable to pass through the diffuser 6150. In this example the diffuser 6150 is positioned in line with the vent body aperture 6138, so that the vent flow of gases leaving the regulated vent flow passage 6132 is immediately incident on the diffuser 6150 as opposed to incident on other surfaces or components. Contacting the diffuser 6150 before contacting other surfaces may advantageously provide for a quieter vent assembly 6000.

As shown in FIGS. 10A-10D, the vent assembly 6000 may comprise a diffuser cover 6152. The diffuser cover 6152 may be attached to a downstream side of the vent body 6130 and may be configured to retain the diffuser in the vent body 6130. In this particular example the vent outlets 6400 are formed in the diffuser cover 6152. In other examples some or all of the vent outlets 6400 may be formed in by the vent body 6130, for example on a peripheral surface of the vent body 6130.

In some examples of the present technology, the vent assembly 6000 may comprise one or more fixed size apertures 6135 defining one or more unregulated vent flow passages 6133 in addition to the regulated vent flow passages 6132. The provision of one or more unregulated vent flow passages 6133 in parallel with the regulated vent flow passages 6132 may advantageously help to tune the vent assembly 6000 to convey gases according to a desired pressure-flow relationship. In some examples the vent assembly 6000 may be tuned to allow a slowly rising flow rate with an increase in pressure. In further examples the vent assembly 6000 may be tuned to allow little or no increase in flow rate corresponding to an increase in pressure.

Furthermore, the fixed sizes apertures 6135 may ensure there are apertures through which gas can flow in the event the regulated gas flow passages 6132 inadvertently become closed or became stuck in a flow-restricting position. In the examples shown in FIGS. 10A-10D there are a plurality of fixed sized apertures 6135 formed through the centre of the plunger 6140, formed proximate to the central axis of the plunger 6140, visible in FIG. 10B.

FIG. 11 shows an exploded view of another vent assembly 6000. In this example the vent assembly 6000 comprises an upstream cover portion 6166 attached to the vent body 6130 in use and configured to cover the membrane 6200 upstream of the membrane 6100. The upstream cover portion 6166 may be generally cylindrical and may have an inner cylindrical surface corresponding to and configured to fit around an outer cylindrical surface of the vent body 6130. The way that the upstream cover portion 6166 may fit to the vent body 6130 is shown in FIG. 13D, which shows another vent assembly 6000 having an upstream cover portion 6166. It is to be understood that the upstream cover portion 6166 may cover an upstream end of the vent body 6130 but may nevertheless have a length that extends to a downstream end of the vent body 6130. The upstream cover portion 6166 may have an upstream face with a plurality of holes formed therein defining inlets to the vent assembly 6000. The upstream cover portion 6166 may function as a cage, allowing gas to flow therethrough but protecting the membrane 6200 and plunger 6140 from damage during use or cleaning. Furthermore, in some examples the upstream cover portion 6166 may help secure the membrane 6200 to the vent body 6130. For example, the membrane 6200 may be secured between the upstream cover portion 6166 and the vent body 6130 or the membrane 6200 may be attached to the upstream cover portion 6616. In some examples the upstream cover portion 6166 is a portion of a wall defining the plenum chamber 3200 of the patient interface 3000, such as a wall of a cushion module 3150. In other examples the vent body 6130 is a portion of a wall defining the plenum chamber 3200 of the patient interface 3000. FIG. 14B shows an example of a vent assembly 6000 provided to a wall of a plenum chamber 3200 in a cushion module 3150. The cushion module 3150 may receive a supply of pressured breathable gas from any suitable air circuit connection (not shown in FIG. 14B), such as a central tube connection either through the vent assembly 6000 as will be described below or separately through the wall of the plenum chamber 3200, or via conduit headgear connections. The vent assembly 6000 shown in FIGS. 10A-10D, 11, 12A-12B or any other vent assembly 6000 described herein may be incorporated into a cushion module 3150 in a similar manner, unless context requires otherwise.

FIGS. 12A and 12B show another example of a vent assembly 6000. In this example the plunger 6140 and membrane 6200 are integrally formed. The membrane 6200 and plunger 6140 may be formed by, for example, injection moulding and may be moulded together in a single shot. In further examples the membrane 6200 and plunger 6140 may be formed by overmoulding, for example by the membrane 6200 being overmoulded to the plunger 6140 or vice versa. The vent assembly 6000 shown in FIGS. 12A and 12B may otherwise generally have the same shape and functions as the vent assembly 6000 shown in FIGS. 10A-10D, including the vent body 6130, the regulated vent flow passage 6132 and frustoconical portions 6134 and 6144 of the vent body 6130 and plunger 6140, respectively. In this example the vent assembly 6000 also comprises a diffuser 6150 and diffuser cover 6152.

4.3.4.10.1 Plunger Vent with Diverging Flow Path

FIGS. 13A-13D show a vent assembly 6000 according to another example of the present technology. This vent assembly 6000 comprises a connecting portion in the form of a membrane 6200, and a plunger 6140 substantially as described with reference to the vent assembly 6000 of FIGS. 10A-10D. Furthermore, the vent assembly 6000 comprises an upstream cover portion 6166 as described with reference to FIG. 11. The vent assembly 6000 in the example shown in FIGS. 13A-13D comprises a vent body 6130 formed by two parts. The plunger 6140 moves with respect to the vent body 6130 to form a regulated vent flow passage 6132 in the same way as described with reference to FIGS. 10A-10D. However, in the FIGS. 13A-13D example the regulated vent flow passage 6132 comprises an upstream portion 6161 and downstream portion 6162. The downstream portion 6162 in this particular example is shaped to have a cross-sectional area that enlarges in the downstream direction. The cross-sectional area may enlarge independently of movement or position of the plunger 6140. That is, the cross section of the downstream portion 6162 of the regulated vent flow passage 6132 may be fixed in shape and size.

The vent body 6130 may comprise an upstream body portion 6133 and a downstream body portion 6134. The upstream body portion 6133 may define the vent body aperture 6138 and the downstream body portion 6134 may at least partially define the downstream portion 6162 of the regulated vent flow passage 6132. The upstream portion 6163 of the vent body 6130 may also partially define the downstream portion 6162 of the regulated vent flow passage 6132. In particular, the vent body 6130 comprises opposing divergent surfaces defining the downstream portion 6162 of the regulated vent flow passage 6132. The divergent surfaces may diverge in the downstream direction such that the cross-sectional area of the downstream portion 6162 of the regulated vent flow passage 6132 increases in the downstream direction. A vent flow passage with an enlarging cross-sectional area prior to release of the flow of gas may advantageously increase the vent flow diffusivity even without the use of a diffuser 6150. The absence of a diffuser 6150 may allow for the vent assembly 6000 to be multi-patient multi-use (MPMU) and may allow for the vent assembly 6000 to be shorter, since there is no space required for housing a diffuser 6150, while the shape of the downstream portion 6162 of the regulated vent flow passage 6132 still provides a diffusing effect. In other examples the vent assembly 6000 may also comprise a diffuser 6150 positioned downstream of the regulated vent flow passage 6132 to provide for additional diffusing.

In the example shown in FIGS. 13A-13D the upstream portion 6161 of the regulated vent flow passage 6132 has a substantially constant cross-sectional area along its length. In particular, the upstream portion 6161 of the regulated vent flow passage 6132 is defined by opposing parallel surfaces of the vent body. Either or both of the upstream portion 6161 and the downstream portion 6162 of the regulated vent flow passage 6132 may be annular in cross section. In this particular example both the upstream portion 6161 and the downstream portion 6162 of the regulated vent flow passage 6132 are annular in cross section.

Furthermore, in the upstream portion 6161 of the regulated vent flow passage 6132 the gas flows partially radially inwardly and in the downstream portion 6162 the gas flows partially radially outwardly. This inward and then outward flow increases the length of the passage through which the gas flows in comparison to a straight passage from one end of the vent assembly 6000 to the other, which may advantageously further reduce velocity of the vent flow and/or noise. Furthermore, the directing the vent flow of gas to flow radially outward after leaving the vent outlets 6400 may advantageously cause further dispersal/diffusing of the vent flow, providing for a quiet vent and low flow velocities after venting of gas.

Further description of vent assemblies 6000 with diverging vent flow of gas is provided below in the Divergent Flow Vent section and is to be read together with the above. Likewise, the disclosure above with reference to FIG. 13A-13D is to be read together with the Divergent Flow Vent section below.

### 4.3.4.10.2 Multiple Membranes and Alternative Housing

FIGS. 14A-14E show another vent assembly 6000 which functions in a similar manner to the vent assembly described with reference to FIGS. 13A-13D and has some of the same parts, such as a vent body 6130, membrane 6200 and plunger 6140 which moves with respect to the vent body 6130 and forms a regulated vent flow path 6132 in a similar way to that disclosed above. One difference, as shown in FIG. 14E in particular, is that the plunger 6140 extends through the vent body aperture 6138. The plunger 6140 in this example partially defines the downstream portion 6162 of the regulated vent flow passage 6132. That said, in this example the spacing between the plunger 6140 and the vent body 6130 in the downstream portion 6162 of the regulated vent flow passage 6132 is sufficiently large that the regulation of the vent flow of gas occurs in the upstream portion 6161 of the regulated vent flow passage 6132 like in the example disclosed with reference to FIGS. 13A-13D. Furthermore, due to the shape of the downstream portion 6162 of the regulated vent flow passage 6132, with increased pressure in the first volume, the plunger 6140 moves away from vent body 6132 at the downstream portion 6162 of the regulated vent flow passage 6132.

In the example shown in FIGS. 14A-14E, the connecting portion which connects the plunger 6140 to the vent body 6130 also differs from the examples shown in FIGS. 10A-13D. As shown in particular in FIGS. 14C-14E, the connecting portion comprises a pair of membranes 6200. The pair of membranes 6200 comprise an upstream membrane 6201 and a downstream membrane 6202. The upstream membrane 6201 is attached to an upstream end of the plunger 6140 and the downstream membrane 6202 is attached to a downstream end of the plunger 6140. Each of the upstream membrane 6201 and the downstream membrane 6202 is connected to the vent body 6130. The upstream membrane 6201 and the downstream membrane 6202 may be connected to the vent body 6130 by being held in place by the vent body 6130 at their peripheries, leaving the central portion of each membrane and the plunger 6140 free to move with respect to the peripheries of each membrane. The upstream membrane 6201, the downstream membrane 6202 and the plunger 6140 may be integrally formed, for example by injection moulding in a single shot. In another example the two membranes may be overmoulded to the plunger 6140. As shown in FIGS. 14C-14E in particular, the vent body 6130 comprises a pair of membrane grooves 6167. The membrane grooves 6167 may be internal grooves configured to receive the respective upstream membrane 6201 and downstream membrane 6202 sufficiently securely that they are held in place in use.

In this example, the vent body 6130 encloses the upstream membrane 6201 and the downstream membrane 6202. The vent body 6130 differs from other examples described thus far in that it is formed in two lateral parts. The vent body 6130 comprises a first lateral side portion 6136 and a second lateral side portion 6137 opposing and connected to the first lateral side portion 6136. The first lateral side portion 6136 and the second lateral side portion 6137 may together define the vent body aperture 6138. The first lateral side portion 6136 and the second lateral side portion 6137 may also together define a circumferential outer surface of the vent body 6130, which may be a substantially cylindrical surface.

The first lateral side portion 6136 and the second lateral side portion 6137 may fit together with dowel pins and corresponding holes, as shown in FIGS. 14C and 14D, or by any other suitable manner of connection. The first lateral side portion 6136 and the second lateral side portion 6137 may be glued or welded together, or may be able to separated for cleaning.

In some examples the first lateral side portion 6136 and the second lateral side portion 6137 may snap fit connect together. FIGS. 15A and 15B show a vent assembly 6000 also having a first lateral side portion 6136 and a second lateral side portion 6137 connected together to form a vent body 6130. In this example the first and second lateral side portions 6136 and 6137 comprise complementary snap fit features configured to enable the first lateral side portion 6136 and the second lateral side portion 6137 to snap fit together. As shown in FIGS. 15A and 15B, the first lateral side portion 6136 comprises snap fit arms 6169 configured snap fit a corresponding receiving portion in the second lateral side portion 6137. This may prevent disassembly or ensure inadvertent disassembly is unlikely or impossible. In some examples the snap fit connection may be substantially permanent.

As shown in FIGS. 14A-14E and FIGS. 15A-15B, the vent body 6130 may comprise an outer groove 6168. The outer groove 6168 may be formed in the circumferential outer surface and may be a groove configured to receive a portion of a patient interface 3000 such as a cushion module 3150 and/or a wall of a plenum chamber 3200, enabling connection of the vent assembly 6000 to the patient interface 3000. The outer groove 6168 may be configured to receive the rim of a hole formed in a wall of the plenum chamber 3200. This also fluidly connects the vent assembly 6000 to the first volume interior to the respiratory therapy system when the first volume is the plenum chamber 3200. FIG. 14B shows the vent assembly 6000 received in a wall of a plenum chamber 3200 of a cushion module 3150. The wall of the plenum chamber 3200 to which the vent assembly 6000 is attached is an anterior wall, and the seal-forming structure of the cushion module 3150 is attached to a periphery of the anterior wall of the plenum chamber 3200 and also partially defines the plenum chamber 3200. The air circuit providing a supply of air or breathable gas to the interior of the plenum chamber 3200 is not shown, but may be a tube connected to the anterior wall of the cushion module 3150, may be a pair of headgear conduit connections on either side of the vent assembly 6000 or may be provide through the centre of the vent assembly 6000 as shown or described with reference to FIGS. 24A-24C below. It is to be understood that, unless the context requires otherwise, any vent assembly 6000 disclosed herein may assembled or incorporated into a patient interface 3000 in the manner shown in FIG. 14B or described as an alternative to FIG. 14B.

4.3.4.10.3 Plunger Vent with Spring

As an alternative to a membrane 6200, a vent assembly 6000 according to one form of the present technology may comprise a spring 6170. FIGS. 16A-16C show a vent assembly 6000 in which the connecting portion comprises a spring 6170 provided between the plunger 6140 and the vent body 6130. Other features of the vent assembly 6000 may be as described with reference to other vent assemblies disclosed herein, such as the regulated vent flow passage 6132, frustoconical portion 6144 of the plunger 6140 and frustoconical portion 6134 of the vent body 6130. In this example the plunger 6140 extends through a vent body aperture 6138 and forms a downstream end 6162 of the regulated vent flow passage 6132 which expands in cross sectional area to provide for diffused flow from the vent outlets 6400.

The spring 6170 in this example comprises a coil spring 6170. The coil spring 6170 may be configured to be compressed in use. As shown in FIG. 16C, the plunger 6140 comprises a central recess and the spring 6170 is positioned within the central recess. The spring 6170 is seated against a downstream end of the vent body 6130. The opposite end of the spring 6170 engages an upstream end of the plunger 6140 at an upstream end of the central recess within the plunger 6140. Upon an increase in pressure in the first volume in use the plunger 6140 may be urged towards the vent body 6130 to reduce a cross sectional area of a regulated vent flow passage 6132. In this particular example the frustoconical portion 6144 of the plunger 6140 is urged towards the frustoconical portion 6134 of the vent body 6130 to adjust a size of the regulated vent flow passage 6132.

In this example the spring 6170 is able to be compressed to allow the plunger 6140 to move towards the vent body 6130, and provides a restoring force to prevent the plunger 6140 from completely occluding the passage and to restore the plunger 6140 to a rest position when there is no longer pressure in the first volume.

FIGS. 17A-17D show a vent assembly 6000 according to another example of the present technology, also comprising a vent body 6130 and plunger 6140 forming a regulated vent flow passage 6132 in a similar manner to other vent assemblies 6000 disclosed herein. In this particular example the connecting portion of the vent assembly 6000 comprises a spring 6170 in the form of a bellows spring 6170.

As illustrated in FIGS. 17C and 17D in particular, the bellows spring 6170 is integrally formed with the plunger 6140. The bellows spring 6170 extends from a downstream side of the plunger 6140 and is seated against a downstream end of the vent body 6130. The downstream end of the vent body 6130 comprises a central portion/hub against which the bellows spring 6170 is seated, and plurality of spokes connecting the central portion to an outer cylindrical portion. Vent outlets 6400 are defined by the spaces between the spokes. The regulated vent flow passage 6132 in this example comprises an upstream portion 6161 and a downstream portion 6162.

The downstream portion 6162 is shaped to have a cross-sectional area that enlarges in the downstream direction independent of movement or position of the plunger 6140. In this particular example, the bellows spring 6170 partially defines the downstream portion 6162 of the regulated vent flow passage 6132. Other features and advantages of a downstream portion 6162 of a regulated vent flow passage 6132 are describe elsewhere herein.

Also as shown in FIGS. 17A-17D, the vent assembly in this example comprises an upstream cover portion 6166 configured to at least partially cover the plunger 6140 upstream of the plunger 6140. The upstream cover portion 6166 retains the plunger 6140 and in this example also limits travel the plunger 6140 away from the vent body 6130. The plunger 6140 comprises a stop 6171 shaped as a rib, on its upstream end face which contacts a downstream-facing surface of the upstream cover portion 6166. The stop 6171 may also space the upstream end face of the plunger 6140 from the upstream cover portion 6166 to enable more of the gas pressure in the first volume to act on the upstream end face of the plunger 6140 increasing the magnitude of the force on the plunger 6140 produced by the gas pressure.

FIGS. 18A-18C show a vent assembly 6000 according to another example of the present technology, being a variation on the vent assembly 6000 shown in FIGS. 17A-17D, the above disclosure of which is applicable to the example of FIGS. 18A-18C with the exception of differences in the upstream cover portion 6166 and vent body 6130. In this example, the vent assembly 6000 comprises an upstream cover portion 6166 configured to at least partially cover the plunger 6140 upstream of the plunger 6140 and comprising a central pin 6172 extending in a downstream direction through a central hole in the plunger 6140 to a downstream end of the vent assembly 6000. The central pin 6172 comprises a flange 6165 positioned at the downstream end of the vent assembly 6000. The bellows spring 6170 in this example may be integrally formed with the plunger 6140 and may be seated against the flange 6165.

FIGS. 20A-20C shows another vent assembly 6000 according to an example of the present technology, advantageously formed by only two parts. In this example the connecting portion connecting the plunger 6140 and the vent body 6130 comprises a spring 6170 in the form of an expandable bellows spring 6170. In this form of the technology, in use, pressure of gas in the first volume causes the expandable bellows spring 6170 to move the plunger 6140 towards the vent body 6170 to regulate the vent flow of gases through a regulated vent flow passage 6132 throughout the therapeutic pressure range. The regulated vent flow passage 6132 may be formed by a vent body aperture 6138 formed at a downstream end of the vent body 6130 between the vent body 6130 and the plunger 6140. As shown in FIG. 20C, the plunger 6140 is positioned adjacent a portion of the vent body 6130 to define the regulated vent flow passage 6132 therebetween. On the opposite side of the plunger 6140 to the vent body 6130 is the expandable bellows spring 6170, arranged such that pressure acting on the interior of the expandable bellows spring 6170 causes the expandable bellows spring 6170 to expand, forcing the plunger 6140 to move towards the vent body 6130 to reduce a cross-sectional area of the regulated vent flow passage 6132 to regulate the vent flow of gases from the first volume to ambient. The first volume is fluidly connected to the interior of the vent assembly 6000 such that it acts on the interior surfaces of the expandable bellows spring 6170.

In the example shown in FIGS. 20A-20C, the vent body aperture 6138 is provided around an outer circumference of the vent body 6130. The plunger 6140 is provided adjacent the outer circumference of the vent body 6130 and, in this particular example, the plunger is annular. The plunger 6140 may be integrally formed with the expandable bellows spring 6170, as shown in FIG. 20C. In this example, the expandable bellows spring 6170 comprises a disc portion 6173 defining an end of the vent assembly 6000 opposite the upstream end of the vent assembly 6000. The disc portion 6173 may be attached to a central pin 6172, shaft or stem, attached to the vent body 6130, for example at the upstream end of the vent assembly 6000 as shown in FIG. 20C. Some or all of the expandable bellows of the expandable bellows spring 6170, plunger 6140, disc portion 6173 and central pin 6172 may be integrally formed. In the example shown in FIGS. 20A-20C all of these portions are integrally formed. The central pin 6172 and disc portion 6173 may be formed to be substantially immovable in use, while the expandable bellows of the expandable bellows spring 6170 are flexible to expand in use under pressure from the first volume, and the plunger 6140 may be moveable due to its connection to the expandable bellows spring 6170. The vent outlet 6400 may be formed by the gap between the vent body 6130 and the plunger 6140, being the same gap the forms the regulated vent flow passage 6132. In some examples, expansion of the bellows spring 6170 is proportional to the pressure in the first volume, enabling the restriction of the flow through the regulated vent flow passage 6132 to be proportional to the therapeutic pressure, providing greater restriction at higher pressure to regulate the vent flow of gas.

### 4.3.4.10.4 Plunger Vent Having Magnets

FIGS. 19A-19C show another form of the present technology in which the vent assembly 6000 comprises a pair of magnetic portions 6175. The vent assembly 6000 comprises many of the same parts and aspects as other examples of the present technology, the description of which can be found elsewhere herein and will not be repeated. These same parts and aspects include a vent body 6130, vent body aperture 6138, plunger 6140, regulated vent flow passage 6132, frustoconical portions 6134 and 6144 of the vent body 6130 and plunger 6140, respectively, and vent outlets 6000. The magnetic portions 6175 may be arranged to provide a similar function to the membrane 6200 or spring 6170 described with reference to other examples herein. That is, the magnetic portions 6175 may be arranged to bias the plunger 6140 towards the rest position but allow the plunger 6140 to move towards the vent body 6130 to reduce a cross-sectional area of the regulated vent flow passage 6132 to regulate a vent flow of gas throughout a therapeutic pressure range. The interior of the vent assembly 6000 may be fluidly connected to a first volume of pressurised gas in use, e.g. formed within a plenum chamber 3200 of a patient interface 3000.

The vent assembly 6000 may comprise a first magnetic portion 6175 and a second magnetic portion 6175. One of the first magnetic portion 6175 and the second magnetic portion 6175 may comprise a magnet and the other of the first magnetic portion 6175 and the second magnetic portion 6175 may comprise a magnet or may comprise a ferromagnetic material. In the example shown in FIGS. 19A-19C, both of the magnetic portions 6175 comprise magnets. In other examples the first magnetic portion 6175 may comprise a magnet and the second magnetic portion 6175 may comprise a ferromagnetic material such as steel/iron, nickel or cobalt. Alternatively the first magnetic portion 6175 may comprise a ferromagnetic material and the second magnetic portion 6175 may comprise a magnet. In the illustrated example the magnets are permanent magnets. In other examples the vent assembly 6000 may comprise one or more electromagnets.

With reference to FIGS. 19A-19C, the first magnetic portion 6175 is supported within the vent assembly 6000 and the second magnetic portion 6175 is attached to the plunger 6140. A magnetic force acts between the first magnetic portion 6175 and the second magnetic portion 6175 biasing the plunger 6140 towards the rest position. In this example, the plunger 6140 moves away from the first magnetic portion 6175 in use to regulate the vent flow of gas through the regulated vent flow passage 6132 and the magnetic force is an attractive force. The first magnetic portion 6175 is positioned upstream of the plunger 6140 in this particular example. In other examples the first magnetic portion 6175 or, more generally, a fixed magnetic portion 6175, may be positioned such that the plunger 6140 moves towards the first magnetic portion 6175 when moving towards the vent body 6130 to regulate the flow of gas. In such an example the magnetic force may be a repulsive force.

Referring again to FIGS. 19A-19C, the vent assembly 6000 comprises an upstream cover portion 6166 attached to the vent body 6130 and configured to cover the plunger 6140 upstream of the plunger 6140. The first magnetic portion 6175 may be retained by the upstream cover portion 6166. As illustrated in FIGS. 19A and 19C for example, the first magnetic portion 6175 is mounted to an upstream side of the upstream cover portion 6166. The second magnetic portion 6175 may be attached or mounted to the plunger 6140. As shown in FIG. 19C for example, the plunger 6140 comprises a central recess, the second magnetic portion 6175 being retained within the central recess.

For avoidance of doubt, a first magnetic portion 6175 and a second magnetic portion 6175 may be provided in a vent assembly 6000 which has a moveable portion which moves to regulate a vent flow of gases, regardless of the form that the moveable portion takes (e.g. it may be a sleeve, membrane or another component). In such an example the moveable portion may be positioned with respect to a vent body 6130 to define a regulated vent flow passage 6132 for the vent flow of gases between the moveable portion and the vent body 6130. The moveable portion may be moveable with respect to the vent body 6130 and may be biased towards a rest position in which the regulated vent flow passage 6132 is open. A first magnetic portion 6175 may be supported within the vent assembly 6000 and a second magnetic portion 6175 may be provided to the moveable portion, a magnetic force acting between the first magnetic portion 6175 and the second magnetic portion 6175 biasing the moveable portion towards a rest position in which the regulated vent flow passage 6132 is open.

The use of magnetic attraction or repulsion to bias a moveable portion advantageously may help keep the moveable portion (e.g. plunger 6140 in the illustrated example) positioned correctly (e.g. centred within the vent assembly 6000). Furthermore, the force of magnetic attraction or repulsion is inversely proportional to the square of the separation of the magnetic portions, whereas the force of a spring or membrane may be directly proportional to displacement of the moveable portion. The use of magnetic forces may allow for the moveable portion to not move at low therapy pressures and instead only begin moving at higher therapy pressure. The vent assembly 6000 may only begin to move upon pressure in the first volume (e.g. a plenum chamber 3200) being reached. This may advantageously keep the regulated vent flow passage as open as possible at lower pressures to ensure ample gas washout when a patient uses only a low therapeutic pressure.

4.3.4.11 Washer Vent with Moveable Edge

Another form of the present technology is shown in FIGS. 21A-21C. The vent assembly 6000 in this form may provide the same function as other vent assemblies 6000 disclosed elsewhere herein. The vent assembly is for a respiratory therapy system for providing respiratory pressure therapy to a patient and is configured in use to convey a vent flow of gases from a first volume interior to the respiratory therapy system to ambient. The vent assembly 6000 may comprise a vent body fluidly connected to the first volume and may define one or more vent outlets.

As shown in FIGS. 21B and 21C in particular, the vent assembly 6000 may comprise an annular membrane 6200 mounted within the vent assembly 6000. A regulated vent flow passage 6132 may be formed between a moveable edge 6205 of the membrane 6200 and a membrane-adjacent portion 6206 of the vent body 6130. In use the vent flow of gases passes through the regulated vent flow passage 6132 from the first volume to the one or more vent outlets 6400. In use, changes in pressure of gas in the first volume cause changes in a position of some or all of the moveable edge 6205 of the membrane 6200 relative to the membrane-adjacent portion 6206 of the vent body 6130 to regulate the vent flow of gases through the regulated vent flow passage 6132 throughout a therapeutic pressure range. In particular, increased pressure in the first volume acts on the side of the membrane 6200 opposite the membrane-adjacent portion 6206 causing the moveable edge 6205 of the membrane 6200 to be urged towards the membrane-adjacent portion 6206. This movement reduces a size of the gap between the moveable edge 6205 and the membrane-adjacent portion 6206 which reduces the cross-sectional area through which the vent flow of gas can pass, maintaining a substantially constant volumetric flow rate or limiting any increase to volumetric flow rate to less than the increase which would occur if the membrane 6200 did not move.

In the example shown in FIGS. 21A-21C, at least some of the vent flow of gases is able to flow from the regulated vent flow passage 6132 along a straight-line path to and through the one or more vent outlets 6400, the straight-line path being unimpeded or impeded only by one or more diffusers 6150. The straight line path is indicated by line SL in FIG. 21C. The gas is able to flow from the regulated vent flow passage 6132 (between the moveable edge 6205 of the membrane 6200 and the membrane-adjacent portion 6206) via a diffuser 6150 directly out a vent outlet 6400 (shown in FIGS. 21A and 21B). It is to be understood that some of the vent flow released from the regulated gas flow passage 6132 may nevertheless be impeded by a portion of the vent assembly 6000, such as in the areas between the vent outlets 4600. It is also to be appreciated that despite being described as flowing in a straight line from the regulated vent flow passage 6132 to a vent outlet 6400, the gas may nevertheless follow a tortuous path through the material of the diffuser 6150, which may a network of fibres, open cell foam or the like. However, paths from the regulated vent flow passage 6132 to the diffuser 6150 and from the diffuser 6150 to the vent outlet(s) 6400 may lie on a straight line.

The vent flow being able to flow along a straight-line path from the regulated vent flow passage 6132 to and through one or more vent outlets 6400 unimpeded or impede only by one or more diffusers 6150 may advantageously provide for a quiet vent, since vent flow being caused to flow onto surfaces forcing a sudden change in direction of the vent flow at the surface may result in noise. A direct unimpeded path (or impeded only by a diffuser) to ambient may result in less noise than a path which is forced to change to direction. The straight-line path may be to one or more vent outlets 6400 from a “pinch point”, being a location at which the cross-sectional area of the vent flow passage is varied due to movement of a membrane 6200. This “pinch point” may be identified as a restriction in the regulated vent flow passage 6132 and may be location along the regulated vent flow passage 6132 at which the cross-sectional area of the flow passage is at a minimum. The vent flow of gas (or at least some of it) may be able to travel in a straight line from location having the smallest cross-sectional area to a vent outlet 4600 unimpeded or impeded only by a diffuser 6150.

In some examples, and as shown in FIGS. 21A-21C, the vent assembly 6000 may also comprise vent outlets 6400 formed in an end portion of the vent body 6130. These vent outlets 6400 may allow some air to exit the vent assembly 6000 without passing through the diffuser 6150 in the event the diffuser 6150 becomes blocked, clogged with fluid etc. These vent outlets 6400 may not be positioned to receive straight-line vent flow from the regulated vent flow passage 6132.

In the example shown in FIGS. 21A-21C, the moveable edge 6205 is an inner edge of the membrane 6200. The membrane 6200 also comprises an outer edge 6207. In other examples the moveable edge 6205 may be an outer edge of the membrane 6200.

As shown in particular in FIG. 21B, the membrane 6200 comprises a frustoconical portion. The frustoconical portion of the membrane 6200 is the free portion, in this example. The membrane 6200 may also comprise a non-frustoconical portion, which may form the outer edge 6207. The non-frustoconical portion may be attached to or lie against an outer portion of the vent body 6130.

The membrane may also comprise a bead formed at the moveable edge 6205. The bead may advantageously have a stabilising effect on the moveable edge 6205, for example by stiffening it. Additionally, or alternatively, as exemplified in FIGS. 21B and 21C, the vent body 6130 may comprise an annular rib forming the membrane-adjacent portion 6206 of the vent body 6130. The rib may function to define a precise location for the membrane-adjacent portion 6206. In some examples the rib may comprise slots defining the regulated vent flow passage 6132 and the rib between the slots may provide for good sealing against the moveable edge 6205 of the membrane 6200, as will be described in more detail below.

FIGS. 22A-22C show a vent assembly 6000 according to another example of the present technology. The vent assembly 6000 in this example operates in a similar manner to the vent assembly 6000 shown in FIGS. 21A-21C. In this example the vent body 6130 comprises a plurality of stops 6171 sized and positioned to limit movement of the moveable edge 6205 of the membrane 6200 in use towards the membrane-adjacent portion 6206 of the vent body 6130, which in this example is formed between the slots 6171. The regulated vent flow passage 6132 may be formed by gaps between the stops 6171, as shown in FIG. 21C in particular. The provision of stops 6171 and gaps between the stops 6171 may help ensure the regulated vent flow passage 6132 remains open and/or the moveable edge 6205 of the membrane 6200 may be particularly stable when it is against the stops 6171. In the example shown in FIGS. 22A-22C the moveable edge 6205 of the membrane 6200, when in contact with the stops 6171, may continue to move towards the membrane-adjacent portion 6206 by deforming between the stops 6171 to reduce the size/area of the openings between the stops 6171, moveable edge 6205 and membrane-adjacent portion 6206 available for the vent flow of gas.

The vent body 6130 in the examples shown in FIGS. 21A-21C and 22A-22C comprises a membrane retainer portion 6180 supporting the outer edge 6207 of the membrane 6200. The vent body 6130 in these examples also comprises a vent cap 6300 attached to the membrane retainer portion 6180. The vent cap 6300 may form the membrane-adjacent portion 6206 of the vent body 6130.

In many examples described herein the vent assembly 6000 comprises a plurality of vent outlets 6400, some of which may open radially outwards as shown in FIGS. 22A-22C for example. Some of the plurality of vent outlets 6400 in this example are defined partially by the membrane retainer portion 6180 of the vent body 6130 and partially by the vent cap 6300. Such vent outlets 6400 may open radially outward. Additionally in this example, some of the plurality of vent outlets 6400 are formed in the vent cap 6300 and are spaced inwardly from an outermost-periphery of the vent body 6130. In other examples of the present technology, the vent cap 6300 may define all or most of the plurality of vent outlets 6400.

As described above, the vent assembly 6000 may comprise a diffuser 6150. Each of the vent assemblies 6000 shown in FIGS. 21A-21C and 22A-22C comprises a diffuser 6150. The diffuser 6150 may be retained between the membrane retainer portion 6180 and the vent cap 6300 and may be positioned such that at least some of the vent flow of gases is able to flow from the regulated vent flow passage 6132 along a direct (e.g. straight-line) path to the diffuser 6150 and then, upon exit from the diffuser 6150, along the direct path to the vent outlets 6400.

It is to be understood that the straight-line path may in practice be a plurality of straight line paths around a circumference of the vent assembly 6000 due to multiple vent outlets 6400. In some examples the straight-line path may in practice be continuous radially outward flow which can be described as forming straight-line paths due to being a straight line when depicted in cross section.

FIGS. 23A-23B show a vent assembly 6000 of another example of the present technology, having the advantage of being a particularly low profile vent assembly 6000. All vent outlets 6400 are formed by the vent cap 6300 in this example, and the vent cap defines the membrane-adjacent portion 6206. The membrane-adjacent portion 6206 is provided directly adjacent the vent outlets 4600. The membrane-adjacent portion 6206 in this example is a single rim formed by the vent cap 6300. The outer edge 6207 of the membrane 6200 is supported proximate the outer periphery of the vent body 6130 and the membrane 6200 extends radially inwardly to position the inner edge of the membrane 6200, being the moveable edge 6205 of the membrane 6200, adjacent the membrane-adjacent portion 6206 to form a regulated vent flow passage 6132 therebetween.

FIGS. 24A and 24B show a vent assembly 6000 according to another example of the present technology. The arrangement of this vent assembly 6000 is similar to the example shown in FIGS. 23A-23B and the manner of operation of the membrane 6200 is similar to that of the examples shown in FIGS. 21A-21C and 22A-22C. One difference in the example shown in FIGS. 24A-24B is that the vent assembly 6000 is configured to fluidly connect to a plenum chamber 3200 of a patient interface 3000 and the vent assembly 6000 comprises an air inlet 6410 configured to receive a pressurised flow of gas at the therapeutic pressure for supply to the plenum chamber 3200 for breathing by the patient. The air inlet 6410 may take the form of a connector configured to connect to an air circuit 4170 to supply the pressurised flow of gas, for example from an RPT device 4000. The vent body 6130 may comprise the connector and the connector may define the air inlet 6410. The connector may be positioned centrally with respect to the vent assembly 6000 and may be configured to project away from the plenum chamber 3200 in use. The connector may be tubular and the supply of gas may flow through a hollow middle of the connector in the direction of a central axis though the connector and into the plenum chamber 3200. A continuous vent flow of gas washing out the plenum chamber 3200 may flow radially outwardly between the moveable edge 6205 of the membrane 6200 and the membrane-adjacent portion 6206 and out the vent outlets 6400. FIG. 24C shows a further variation in which the vent assembly 6000 comprises a heat and moisture exchanger (HMX) 6420 attached to the vent body 6310 in use. The HMX 6420 may be positioned between the plenum chamber 3200 and the vent body 6310 such that exhalate accumulating in the plenum chamber 3200 must pass through the HMX 6420 prior to reaching the regulated vent flow passage 6132, depositing moisture and heat within the HMX 6420. The supply of air from the air inlet 6410 must also then pass through the HMX 6420 before reaching the plenum chamber 3200, receiving some of the moisture and heat from the HMX 6420. The HMX 6420 may be a commercially available model or may be as known in the art.

FIGS. 29A-29D show another example of the present technology in the form of a vent assembly 6000 similar to the vent assemblies 6000 shown in FIGS. 21A-21C. 22A-22C and 23A-23B. One difference in the vent assembly 6000 shown in FIGS. 29A-29D is that the moveable edge 6205 of the annular membrane 6200 is an outer edge of the membrane 6200. The membrane 6200 also comprises an inner edge in this example although in other examples the membrane 6200 may be in the form of a disc whereby there is no inner edge. In the illustrated example the vent body 6130 comprises a membrane retainer portion 6180 supporting the inner edge of the membrane 6200.

The membrane retainer portion 6180 in this example forms the membrane-adjacent portion 6206 of the vent body 6130. In particular, the membrane-adjacent portion 6206 may be formed by an inner peripheral edge of the membrane retainer portion 6180. In this example, the membrane retainer portion 6180 comprises a cylindrical portion forming the periphery of the membrane retainer portion 6180 (and also forming the periphery of the vent body 6130) and, as shown in FIGS. 29C and 29D in particular, the membrane retainer portion 6180 may comprise a corner formed on the inside of the membrane retainer portion 6180 around an inside circumference forming the membrane-adjacent portion 6206. The corner may be rounded as shown in FIGS. 29C and 29D. The moveable edge 6205 of the membrane 6200 may be positioned proximate the membrane-adjacent portion 6206 (e.g. the corner) to form the regulated vent flow passage 6132 therebetween and may move towards the membrane-adjacent portion 6206 in response to increasing pressure in the first volume, to reduce a cross-sectional area through which the vent flow of gas can pass, to prevent or limit increases in volumetric flow rate through the regulated vent flow passage 6132, to regulate the vent flow of gas throughout a therapeutic pressure range.

In the example shown in FIGS. 29A-29D, the vent assembly 6000 comprises a plurality of vent outlets 6400. In this example some of the vent outlets 6400 open radially outwards and some open in a direction parallel to a central axis of the membrane 6200 (which in this example is also a central axis of the vent assembly 6000 and other components thereof). Vent outlets 6400 which open radially outward may be formed by the membrane retainer portion 6180, for example as shown in FIGS. 29A-29D. The vent body 6130 in this example comprises a vent cap 6300 attached to the membrane retainer portion 6180. The vent cap 6300 may form at least some of the vent outlets 6400, such as the vent outlets 6400 which open in a direction parallel to a central axis of the membrane 6200, which may also be in a direction away from the first volume, away from plenum chamber 3200 and/or away from the patient in use.

As shown in FIGS. 29A-29D, the vent assembly 6000 may comprise an annular membrane cover 6182. The annular membrane cover 6182 may comprise an annular portion and a plurality of radially outwardly projecting portions extending from the annular portion. The annular portion of the annular membrane cover 6182 may fit to a correspondingly sized cylindrical portion of the membrane retainer portion 6180 or other portion of the vent body 6130. The inner edge of the membrane 6200 in the example shown in FIGS. 29A-29D may be held between the membrane retainer portion 6180 and the annular membrane cover 6182.

Another feature of the vent assembly 6000 shown in FIGS. 29A-29D is that, like some other examples disclosed herein, the vent assembly 6000 defines one or more regulated vent flow passages 6132 and one or more unregulated vent flow passages 6133. For example, the vent assembly 6000 may comprise one or more fixed size apertures 6135 defining one or more unregulated vent flow passages 6133 in addition to the regulated vent flow passages. As shown in FIG. 29C in particular, the vent assembly 6000 comprises a plurality of fixed size apertures 6135 formed through the vent body 6130, fluidly connected to the first volume and located inwardly of the membrane 6000. The fixed size aperture 6135 may be formed in the membrane retainer portion 6180. This vent assembly 6000 also advantageously enables at least some of the vent flow of gases to flow from the regulated vent flow passage 6132 along a straight-line path SL (depicted in FIG. 29C) to and through one or more vent outlets 6400, the straight-line path being impeded only by one or more diffusers. In alternative examples the straight-line path is unimpeded, for example if there is no diffuser incorporated into the vent assembly 6000.

4.3.4.12 Washer Vent Having Membrane with Apertures

FIGS. 25A-25C show a vent assembly 6000 being another example of the present technology. The vent assembly 6000 is for a respiratory therapy system for providing respiratory pressure therapy to a patient and is configured in use to convey a vent flow of gases from a first volume interior to the respiratory therapy system to ambient. The vent assembly 6000 comprises a vent body 6130 fluidly connected to the first volume and defining a plurality of vent outlets 6400 (in other examples there may only be one vent outlet 6400).

In this example the vent assembly 6000 comprises an annular membrane 6200. The annular membrane 6200 may be mounted within the vent assembly 6000 and may comprise an inner edge 6207 and an outer edge 6208. The membrane 6200 may further comprise at least one membrane aperture 6210 formed in the membrane 6200 between the inner edge 6207 and the outer edge 6208. As shown in FIG. 25A for example, the membrane 6200 comprises three membrane apertures 6200. A first regulated vent flow passage 6132a may be formed between a first membrane-adjacent portion 6206a of the vent body 6130 and a membrane aperture 6210, and the vent flow of gases is able to pass through the first regulated vent flow passage 6132a during flow from the first volume to the one or more vent outlets 6400. In use, changes in pressure of gas in the first volume may cause changes in a position of the membrane aperture(s) 6210 relative to the first membrane-adjacent portion 6206a of the vent body 6130 to regulate the vent flow of gases through the first regulated vent flow passage 6132a throughout a therapeutic pressure range.

In the example shown in FIGS. 25A-25C, a first regulated vent flow passage 6132a is formed through each membrane aperture 6210. The vent body 6130 comprises a first membrane-adjacent portion 6206a positioned over each of the membrane apertures 6210 to form the first regulated vent flow passage 6132a. When pressure the first volume increases in use, the membrane 6200 may deform such that the membrane apertures 6210 move closer to the first membrane-adjacent portion 6206a, reducing the available cross-sectional area for the vent flow of gas to pass, preventing or at least limiting increases in volumetric flow rate through the first regulated vent flow passage 6132a.

The one or more vent outlets 6400 may comprise at least one annular vent outlet 6400. As shown in FIGS. 25A-25C for example, the vent assembly 6000 comprises a pair of annular vent outlets 6400. The two annular vent outlets 6400 are concentric with each other. The first membrane-adjacent portion 6206a in this example is provided between the pair of annular vent outlets 6400. In this example, the portion of the vent body 6130 between the annular vent outlets 6400 defines the first membrane-adjacent portion 6206a between the vent outlets 6400.

The membrane 6200 may comprise a plurality of membrane apertures 6200 spaced along a circumference of the membrane 6200. As shown in FIG. 25A in particular the three membrane apertures 6200 are each spaced along a single circumference (e.g. they are at the same radius from a central axis through the membrane 6200). The membrane apertures 6210 are equally spaced around the circumference on which they are formed, in this example. The circumference of the membrane 6200 along which the membrane apertures 6200 are spaced is centrally located between the inner edge 6208 and the outer edge 6207 of the membrane 6200, as shown in FIGS. 25A-25C. In this example each of the plurality of membrane apertures 6210 comprises a slit. Each slit may be aligned along the circumference of the membrane 6200 along which the membrane apertures 6210 are spaced.

In some alternative examples, the membrane apertures 6210 may circular, ovals, or any other shapes. For example, each membrane aperture 6210 may in some examples be a circular hole.

In some examples, in addition to one or more membrane apertures 6210 which move with respect to a membrane-adjacent portion 6206, one of the inner edge 6208 and the outer edge 6207 may be a moveable edge 6205 of the membrane 6200. The vent assembly 6000 in FIG. 25A-25C is one such example. A second regulated vent flow passage 6132b may be formed between a second membrane-adjacent portion 6206b of the vent body 6130 and the moveable edge 6205 of the membrane 6200, the vent flow of gases being able to pass through the second regulated vent flow passage 6132b during flow from the first volume to the one or more vent outlets 6400. In use, changes in pressure of gas in the first volume may cause changes in a position of some or all of the moveable edge 6205 of the membrane 6200 relative to the second membrane-adjacent portion 6206b of the vent body 6130 to regulate the vent flow of gases through the second regulated vent flow passage 6132b throughout a therapeutic pressure range.

The moveable edge 6205 and second membrane-adjacent portion 6206b may function in the same or a similar manner to the moveable edge 6205 and membrane-adjacent portion 6206 in other examples of vent assemblies 6000 disclosed herein having annular membranes 6200 without membrane apertures 6210, such as shown in FIGS. 21A-21C, 22A-22C, 23A-23B, 24A-24C, and 29A-29D. The disclosure made with reference to those examples is to be understood as relevant to the operation of the moveable edge 6205 and second membrane-adjacent portion 6206b of the vent assembly 6000 shown in FIGS. 25A-25C.

At least below a predetermined pressure, the vent flow of gas may pass through the first regulated vent flow passage 6132a and the second regulated vent flow passage 6132b in parallel. However, in some examples upon pressure in the first volume exceeding the predetermined pressure, the second regulated vent flow passage 6132b closes. This is because the moveable edge 6205 may contact and seal against the second-membrane adjacent portion 6206b of the vent body 6130 and prevent flow. This two-stage regulation of vent flow may advantageously allow for ample vent flow rate at lower therapeutic pressures, ensuring the vent assembly 6000 is at least sufficiently open to maintain sufficient gas washout even at low therapeutic pressures, but may also allow for complete closing of the first regulated vent flow passage 6132a at higher therapeutic pressures when only a single (second) regulated vent flow passage 6132b is required. FIGS. 25B and 25C show the first regulated vent flow passage 6132a and second regulated vent flow passage 6132b.

Referring to FIGS. 25A-25C, the vent body 6130 in this example may comprise a cylindrical outer portion supporting the outer edge 6207 of the annular membrane 6200 and a cylindrical inner portion forming the second membrane-adjacent portion 6206b of the vent body 6130. The vent outlets 4600 may be formed between the cylindrical outer portion and the cylindrical inner portion.

In a variation of the example shown in FIGS. 25A-25C, the vent assembly 6000 may comprise a diffuser 6150 positioned over the vent outlets 6400, for example retained by a diffuser cover 6152. In another variation the vent outlets 6400 may be formed by diverging opposing surfaces to help diffuse the vent flow of gas as it is emitted from the vent assembly 6000. In a further variation the vent body 6130 may comprise a membrane cover 6182, for example at the upstream end of the membrane 6200, to provide further support and protection for the membrane 6200.

FIGS. 26A-26C show another vent assembly 6000 according to another example of the present technology. In this example the vent body 6130 comprises a vent base 6100 on which a membrane 6200 having a plurality of membrane apertures 6200 is supported, and a vent cap 6300 defining the first membrane-adjacent portion 6206 of the vent body 6130. In this example there may be no second membrane-adjacent portion 6206b and the vent flow of gas may pass through the membrane apertures 6200 only, and not past the inner edge 6208 and the outer edge 6207 of the membrane 6200. At least one of the vent outlets 6400 may be formed in the vent cap 6300. Alternatively or additionally, at least one of the vent outlets 6400 may be defined by a spacing between a peripheral edge of the vent cap 6300 and an outer edge of the membrane 6200 (which may be an immovable edge). For example, as shown in FIGS. 26B-26C, there are vent outlets 6400 opening in a direction parallel to a central axis of the vent assembly 6000 as well a vent outlet 6400 opening radially outward. In this example the vent outlet 6400 opening radially outward is a single continuous vent outlet 6400 extending around an entire circumference of the vent assembly 6000. The vent outlets 6400 formed in the vent cap 6300 are not shown in FIG. 26A, for clarity.

FIGS. 27A-27E show another example of the vent assembly 6000 of the present technology. The membrane has membrane apertures 6210 and the operation of the membrane 6200 is similar to that of the example of FIGS. 26A-26C and will not be repeated in as much detail. One difference is that while in the FIGS. 26A-26C example the membrane-adjacent portion 6206 is continuous around a circumference of the vent assembly 6000, in the FIGS. 27A-27E example the membrane-adjacent portion 6206 comprises a plurality of discrete surfaces separated by openings 6209 (shown in FIG. 27C in particular) through which the vent flow of gas is able to flow after passing through the membrane apertures 6210. Furthermore, in this example the vent assembly 6000 comprises a diffuser 6150 between a regulated vent flow passage 6132 and the vent outlet 6400.

As mentioned above, a vent assembly 6000 with forming a regulated vent flow passage 6132 may also comprise one or more fixed size apertures defining one or more unregulated vent flow passages 6213. As shown in FIGS. 27C and 27D in particular, in this example the vent assembly 6000 comprises a plurality of fixed size apertures 6135 positioned inwardly of the membrane 6200, bypassing the membrane 6200. As illustrated, the fixed size apertures 6135 are formed in the vent body 6130 in positions radially inward of the inner edge 6208.

### 4.3.4.13 Divergent Flow Vent

FIGS. 28A-28D show another vent assembly 6000 according to an example of the present technology. Like other examples of the present technology the vent assembly 6000 is for a respiratory therapy system for providing respiratory pressure therapy to a patient and is configured in use to convey a vent flow of gases from a first volume interior to the respiratory therapy system to ambient. In this example the vent assembly 6000 comprises a vent body 6130 fluidly connected to the first volume and defining a vent flow passage 6131 through which the vent flow of gases is able to flow from the first volume to ambient. In this example, the vent flow passage 6131 comprises an upstream portion 6191 and a downstream portion 6192. At least the downstream portion 6192 may be annular in cross section and may be shaped to have a cross sectional area that enlarges in the downstream direction.

With reference to FIGS. 28A-28D, the vent body 6130 comprises opposing divergent surfaces 6195 defining the downstream portion 6192 of the vent flow passage 6131. The divergent surfaces 6195 diverge in the downstream direction such that the cross-sectional area of the downstream portion 6192 of the vent flow passage 6131 increases in the downstream direction. Advantages of divergent surfaces and a vent flow passage which increases in cross-sectional area are discussed above in relation to FIGS. 13A-13D, the disclosure of which is to be considered relevant to the example shown in FIGS. 28A-28D.

In the FIGS. 28A-28D example the upstream portion 6191 of the vent flow passage 6131 has a substantially constant cross-sectional area along its length. The upstream portion 6191 may be defined by opposing parallel surfaces of the vent body. The opposing parallel surfaces may be frustoconical surfaces. As illustrated in FIG. 28D in particular, the upstream portion 6191 of the vent flow passage 6131 is shaped such that the vent flow of gases flows partially radially inwardly along the length of the upstream portion 6191 in a downstream direction. The downstream portion 6192 of the vent flow passage 6131 is shaped such that the vent flow of gases flows partially radially outwardly along the length of the downstream portion 6192 in the downstream direction. This arrangement emits the vent flow of gas radially outwardly, advantageously encouraging further diffusivity of the gas after leaving the vent assembly 6000. The radially inward and the outward flow through the vent flow passage 6131 lengthens the flow passage in comparison to a straight flow passage from one end of the vent assembly 6000 to the other, which may advantageously provide for lower peak flow velocity and quiet operation. These aspects may provide for quiet operation without a diffuser 6150, enabling the vent assembly 6000 to be used in multi-patient multi-use (MPMU) applications. The lack of diffuser also provides for a smaller vent assembly 6000 with fewer components than if a diffuser was included.

The downstream portion 6192 of the vent flow passage 6131 is defined by opposing non-parallel surfaces 6195. Each of the non-parallel surfaces 6195 in the illustrated example extends radially outwardly in the downstream direction, which helps to discharge the vent flow of gas outwardly from the vent assembly 6000, encouraging further diffusivity. In this example each of the non-parallel surfaces 6195 is frustoconical.

In the example shown in FIGS. 28A-28D, the vent body 6130 comprises a central portion 6193 and a peripheral portion 6194. The downstream portion 6192 of the vent flow passage 6131 is defined between the central portion 6193 and the peripheral portion 6194. In this example the vent body 6130 comprises an upstream cover portion 6166 which may connect between the central portion 6193 and the peripheral portion 6194 and may support the central portion 6193 within the peripheral portion 6194. The upstream portion 6191 of the vent flow passage 6131 may be defined between the upstream cover portion 6166 and the peripheral portion 6194. The provision of a central portion 6193 and peripheral portion 6194 joined at the upstream end enables a sing vent outlet 6400 to be formed at the lower end around a full circumference of the vent assembly 6000, as shown in FIG. 28A. The vent flow can advantageously flow unimpeded through the vent outlet 6400 since no components or portions are within the gas flow path through and out of the downstream portion 6192, which may provide for quiet operation.

### 4.3.5 Decoupling Structure(s)

In one form the patient interface 3000 includes at least one decoupling structure, for example, a swivel or a ball and socket. In some forms, the decoupling structure may be part of an elbow structure or connection member located between, and configured to fluidly connect, the plenum chamber 3200 and the air circuit 4170.

### 4.3.6 Connection Port

Connection port 3600 allows for connection to the air circuit 4170.

### 4.3.7 Forehead Support

In one form, the patient interface 3000 includes a forehead support 3700.

### 4.3.8 Anti-Asphyxia Valve

In one form, the patient interface 3000 includes an anti-asphyxia valve (AAV). In other forms, the AAV may be located in an elbow structure or connection member located between the plenum chamber 3200 and the air circuit 4170. Alternatively, the AAV may be located in a part of the conduit headgear, for example tubes 3350. An AAV may alternatively be referred to as a non-rebreathing valve (NRV).

### 4.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 4.4.1 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 4.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

### 4.6 Humidifier

### 4.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 4.8 Respiratory Therapy Modes

Various respiratory therapy modes may be implemented by the disclosed respiratory therapy system.

### 4.9 Glossary

For the purposes of the present technology disclosure, in certain forms of the present technology, one or more of the following definitions may apply. In other forms of the present technology, alternative definitions may apply.

### 4.9.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m2=1 millibar≈0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.9.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240

Polycarbonate: a thermoplastic polymer of Bisphenol-A Carbonate.

### 4.9.2 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.11 Reference Signs List

1000 Patient
1100 Bed partner
3000 Patient interface
3100 Seal-forming structure
3200 Plenum chamber
3300 Positioning and stabilising structure
3310 Rigidiser arms
3400 Vent
3600 Connection port
3700 Forehead support
3800 Cannula
3810a Nasal prongs
3810b Nasal prongs
3820a Lumens
3820b Lumens
4000 RPT device
4010 External housing
4012 Upper portion
4014 Lower portion
4015 Panel(s)
4016 Chassis
4018 Handle
4020 Pneumatic block
4100 Pneumatic components
4110 Air filters
4112 Inlet air filter
4114 Outlet air filter
4120 Muffler
4122 Inlet muffler
4124 Outlet muffler
4140 Pressure generator
4142 Blower
4144 Brushless DC motor
4160 Anti-spill back valve
4170 Air circuit
4180 Supplemental oxygen
4200 Electrical components
4202 Printed Circuit Board Assembly (PCBA)
4210 Power Supply
4220 Input devices
4270 Transducers
5000 Humidifier
5002 Humidifier inlet
5004 Humidifier outlet
5006 Humidifier base
5110 Reservoir
5120 Conductive portion
5130 Humidifier reservoir dock
5135 Locking lever
5150 Water level indicator
5240 Heating element
6000 Vent assembly
6100 Vent base
6105 Downstream housing
6110 Vent base aperture
6120 Upper region
6130 Vent body
6131 Vent flow passage
6132 Regulated vent flow passage
6133 Unregulated vent flow passage
6134 Frustoconical portion of the vent body
6135 Fixed size aperture
6136 First lateral side portion of the vent body
6137 Second lateral side portion of the vent body
6138 Vent body aperture
6140 Plunger
6144 Frustoconical portion of the plunger
6150 Diffuser
6152 Diffuser cover
6161 Upstream portion of the regulated vent flow passage
6162 Downstream portion of the regulated vent flow passage
6163 Upstream portion of the vent body
6164 Downstream portion of the vent body
6165 Flange
6166 Upstream cover portion
6167 Membrane groove
6168 Outer groove
6169 Snap fit arms
6170 Spring
6171 Stop
6172 Central pin
6173 Disc portion
6175 Magnet
6180 Membrane retainer portion
6182 Membrane cover
6191 Upstream portion of the vent flow passage
6192 Downstream portion of the vent flow passage
6193 Central portion of the vent body
6194 Peripheral portion of the vent body
6195 Non-parallel surfaces
6200 Membrane
6201 Upstream membrane
6202 Downstream membrane
6205 Moveable edge
6206 Membrane-adjacent portion
6207 Outer edge
6208 Inner edge
6210 Membrane aperture
6220 Non-planar portion
6230 Cylindrical portion
6240 Flange
6250 Rib sections
6300 Vent cap
6301 Vent cap holes
6302 Central hole
6310 First surface
6320 Second surface
6330 Cap apertures
6400 Vent outlet(s)
6410 Air inlet
6420 Heat and moisture exchanger
6500 Connector portion
6600 Vent gap
6700 Plurality of flaps
6701 Fixed end
6702 Free end
6710 Plurality of slits
6800 Central aperture

1.-91. (canceled)

92. A vent assembly for a respiratory therapy system, the vent assembly being configured in use to convey a vent flow of gases exhaled by a patient from a first volume interior to the respiratory therapy system to ambient, the vent assembly comprising:
a vent base having formed therein a vent base aperture;
a flexible membrane mounted within the vent assembly and spanning across the vent base aperture, wherein the membrane has formed therein a membrane aperture to allow the vent flow to pass therethrough; and
a vent cap connected to the vent base, wherein the vent cap is located downstream of the membrane relative to the vent flow, and wherein the vent cap is positioned in the path of the vent flow through the membrane aperture;
wherein, in use, the pressure of gas in the first volume acts on the membrane such that changes in the pressure of the gas in the first volume causes the membrane to flex thereby varying a position of the membrane relative to the vent cap in order to control the vent flow through one or more vent outlets to ambient;
wherein the membrane comprises a non-planar portion having a first side facing towards the first volume in use and a second side facing away from the first volume in use;
wherein, at least in the absence of a pressure difference between the first volume and ambient, the first side of the non-planar portion is concave and the second side of the non-planar portion is convex.

93. The vent assembly according to claim 92, wherein the non-planar portion is substantially dome-shaped.

94. The vent assembly according to claim 92, wherein a central region of the membrane comprises the membrane aperture and non-central regions of the membrane are impermeable to gas.

95. The vent assembly according to claim 92, wherein the vent cap is mounted to the vent base to form the one or more vent outlets between the vent cap and the vent base around a periphery of the vent cap.

96. The vent assembly according to claim 92, wherein the one or more vent outlets are formed as a plurality of apertures in the vent cap and wherein the membrane and vent cap are configured so that flexing of the membrane varies the number of the plurality of apertures that are blocked by the membrane to restrict the vent flow of gas to ambient therethrough.

97. The vent assembly according to claim 96, wherein the membrane and vent cap are configured so that, as flexing of the membrane increases, apertures of the plurality of apertures located closer to a periphery of the vent cap are blocked by the membrane prior to apertures of the plurality of apertures located further from the periphery of the vent cap.

98. The vent assembly according to claim 96, wherein the membrane aperture is one of a plurality of membrane apertures formed in the membrane to allow the vent flow to pass therethrough.

99. The vent assembly according to claim 92, wherein the vent cap is sealingly mounted to the vent base around a periphery of the vent cap.

100. The vent assembly according to claim 92, wherein a side of the vent cap facing towards the membrane has a shape corresponding to a shape of the membrane when flexed.

101. The vent assembly according to claim 92, wherein the membrane is mounted to the vent base around a perimeter of the membrane.

102. The vent assembly according to claim 101, wherein the membrane is sealingly mounted to the vent base around the perimeter of the membrane.

103. The vent assembly according to claim 92, wherein the vent assembly is configured to form part of a patient interface.

104. The vent assembly according to claim 103, wherein the vent base is configured to connect to a portion of a plenum chamber of the patient interface.

105. The vent assembly according to claim 92, wherein the vent assembly is configured such that a vent flow rate of the vent flow of gases from the first volume to ambient is substantially constant for a range of pressures inside the first volume.

106. A patient interface comprising:
a plenum chamber pressurisable to a therapeutic pressure of at least 6 cmH2O above ambient air pressure, said plenum chamber including a plenum chamber inlet port sized and structured to receive a flow of air at the therapeutic pressure for breathing by a patient,
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use; and
a vent assembly configured to convey a vent flow of gases exhaled by the patient from an interior of the plenum chamber to ambient, said vent assembly being sized and shaped to maintain the therapeutic pressure in the plenum chamber in use,
wherein the patient interface is configured to allow the patient to breath from ambient through their mouth in the absence of a flow of pressurised air through the plenum chamber inlet port, or the patient interface is configured to leave the patient's mouth uncovered; and
wherein the vent assembly comprises:
a vent base having formed therein a vent base aperture;
a flexible membrane mounted within the vent assembly and spanning across the vent base aperture, wherein the membrane has formed therein a membrane aperture to allow the vent flow to pass therethrough; and
a vent cap connected to the vent base, wherein the vent cap is located downstream of the membrane relative to the vent flow, and wherein the vent cap is positioned in the path of the vent flow through the membrane aperture;
wherein, in use, the pressure of gas in the plenum chamber acts on the membrane such that changes in the pressure of the gas in the plenum chamber causes the membrane to flex thereby varying a position of the membrane relative to the vent cap in order to control the vent flow through one or more vent outlets to ambient;
wherein the membrane comprises a non-planar portion having a first side facing towards the plenum chamber in use and a second side facing away from the plenum chamber in use;
wherein, at least in the absence of a pressure difference between the plenum chamber and ambient, the first side of the non-planar portion is concave and the second side of the non-planar portion is convex.

107. The patient interface of claim 106, wherein the patient interface comprises a cushion module comprising the seal-forming structure, the cushion module and the vent assembly together forming the plenum chamber.

108. The patient interface of claim 107, wherein patient interface comprises two vent assemblies respectively provided left and right sides of the cushion module.

109. The patient interface of claim 108, wherein the vent assemblies each connect to a positioning and stabilising structure of the patient interface.

110. The patient interface of claim 109, wherein the vent assemblies each connect to a respective one of a pair of rigidiser arms of the positioning and stabilising structure.

111. The patient interface of claim 106, wherein the vent assembly provided to a decoupling structure of the patient interface.
